(19) United States
(12) Patent Application Publication
(10) Pub. No.: US 2024/0033374 A1
(43) Pub. Date: Feb. 1, 2024

(54) Nano-structural Protein Degradation Tool, Use, and Preparation Method thereof, and Lipid-based Protein Degradation Tool, Use, and Preparation Method thereof

(71) Applicant: HENAN UNIVERSITY, Kaifeng (CN)

(72) Inventors: Bingyang SHI, Kaifeng (CN); Meng ZHENG, Kaifeng (CN); Yang LIU, Kaifeng (CN); Runhan LIU, Kaifeng (CN)

(21) Appl. No.: 18/223,962

(22) Filed: Jul. 19, 2023

(30) Foreign Application Priority Data
Jul. 29, 2022 (CN) 2022109065646
Jul. 29, 2022 (CN) 2022109118535

(51) Int. Cl. A61K 47/69; A61K 47/68; A61K 47/54

(52) U.S. Cl. CPC A61K 47/6931; A61K 47/6893; A61K 47/543

(57) ABSTRACT

The present disclosure provides a nano-structural protein degradation tool, use, and a preparation method thereof, wherein the nano-structural protein degradation tool comprises: one of or a combination of several of a first degradation tool, a second degradation tool, and a third degradation tool, wherein the first degradation tool is formed by linking POI recognition groups to linkers; the second degradation tool is formed by linking the POI recognition groups to nanoparticles; and the third degradation tool is formed by linking the POI recognition groups to the nanoparticles through the linkers. The present disclosure further provides a lipid-based protein degradation tool, use, and a preparation method thereof, wherein the lipid-based protein degradation tool comprises: POI recognition groups, and lipid hybrid substances linked to the POI recognition groups.

### CROSS-REFERENCE TO RELATED APPLICATIONS

The present disclosure claims the priority to the Chinese patent application filed with the China National Intellectual Property Administration on Jul. 29, 2022 with the filing No. 202210906564.6 and entitled “Nano-structural Protein Degradation Tool, Use, and Preparation Method thereof”, and the Chinese patent application filed with the China National Intellectual Property Administration on Jul. 29, 2022 with the filing No. 202210911853.5, and entitled “Lipid-based Protein Degradation Tool, Use, and Preparation Method thereof”, all the contents of which are incorporated herein by reference in entirety.

### TECHNICAL FIELD

The present disclosure belongs to the technical field of targeted drugs, and in particular to a nano protein degradation tool, use, and a preparation method thereof, and a lipid-based protein degradation tool, use, and a preparation method thereof.

### BACKGROUND ART

The targeted protein degradation (TPD) tool specifically hijacks a protein of interest (POI) to an intracellular protein recycling machinery to achieve targeted protein degradation. TPD has become a powerful tool in biomedical research and pharmaceutical industries.

Compared with the conventional enzyme inhibiting/antagonizing small molecule drugs, proteolytic targeting chimera (PROTAC), as a representative member of the most representative TPD of the first generation, is capable of targeted degrading conventionally undruggable targets, but targets are limited only to intracellular proteins. Extracellular proteins and membrane proteins play an important role in the occurrence and development of diseases, with approximately 40% of the total gene-encoded proteins being non-intracellular proteins. In order to expand the scope of target proteins degradation to the non-cytosolic targets, a research team has developed lysosome-targeting chimaeras (LYTAC), which links antibody/peptide by cationic phospho-oligomannose tail, and enables lysosome to exert the degradation effect after endocytosis with the aid of the mannose-6-phosphate receptor. Subsequently, the team utilized the characteristic that hepatocytes are rich in asialoglycoprotein receptor (ASGPR) to replace LYTAC mannose phosphate tail with a triantenerrary N-acetylgalactosamine structure, and further upgraded the LYTAC to liver-specific LYTAC. Although the LYTAC method has medicinal application potential, the synthesis method is complex. LYTAC is receptor-dependent and needs to be specially designed in the LYTAC structure to help protein complex enter cells, and meanwhile, for different cell targeting, the tail needs to be changed case-by-case, so that it is tedious and difficult in design and synthesis for different diseases and diseases tissue.

For successful development of current well-recognized TPD tools, (1) a suitable target protein recognition group needs to be designed (2) receptor-ligand matching pairs for hijacking proteins into cells and intracellular trafficking is required, (3) an appropriate protein degradation mechanism need to be designed, and (4) from the perspective of translational medicine, it is necessary to design the tissue/cell targeting capacity and biological barriers penetrating capacity for cell type specificity. However, all of existing TPD tools including PROTAC, LYTAC and similar designing examples require a laborious design for individual cases when developing a tool for any new POI, in which de novo synthesis and laborious screening is required for different diseases and cell types.

To sum up, the existing targeted protein degradation tools have the challenges of complex design and laborious synthesis methods, requiring case-by-case design for each disease and cell type, poor structural flexibility, modification inconvenience, poor in vivo targeting, and having no biological barriers penetrating capacity such as blood-brain barrier, and challenged by targeting treatment for patients.

### SUMMARY

In order to solve the abovementioned problems, the present disclosure provides a nano-structural protein degradation tool, including: one of or a combination of several of a first degradation tool, a second degradation tool, and a third degradation tool;
in the above, the first degradation tool is formed by linking protein-of-interest (POI) recognition groups to linkers; the second degradation tool is formed by linking the POI recognition groups to a nanoparticle; and the third degradation tool is formed by linking the POI recognition groups to the nanoparticle through the linkers; and
the POI recognition groups include antibodies, proteins, peptides, nucleic acid aptamers or small molecules capable of specifically binding to the POI.

Optionally, in the first degradation tool, the POI recognition group and the linker constitute a set of linking unit; and the first degradation tool is the nano-structural protein degradation tool having a multi-layer structure and composed of a plurality of sets of linking units, wherein the linking unit comprises the linker located at a core and the POI recognition group linked to the linker and located at periphery;
the second degradation tool is the nano-structural protein degradation tool having a multi-layer structure and composed of the nanoparticle located at the core and a plurality of the POI recognition groups linked to the nanoparticle and located at periphery; and
in the third degradation tool, the POI recognition groups are linked to the nanoparticle through the linkers; and the third degradation tool is the nano-structural protein degradation tool having a multi-layer structure and composed of the nanoparticle located at the core and a plurality of sets of linking units, wherein the linking unit comprises the linker linked to the nanoparticle and located at an intermediate layer, and the POI recognition group located at periphery and linked to the linker.
optionally, the linkers comprise hydrophilic polymers, hydrophobic polymers, and amphiphilic polymers;
optionally, the linkers have molecular weights of 0-1000 kDa, not including 0 kDa; and
optionally, the amphipathic polymers comprise: amphipathic block co-polymers, amphipathic polymers formed by the hydrophilic polymers and hydrophobic small molecules, and amphipathic polymers formed by the hydrophobic polymers and hydrophilic small molecules.

Optionally, the amphiphilic polymers are polymers of chain-like or branched molecular structures, which have at least one hydrophilic molecular terminal and one hydrophobic molecular terminal; and
optionally, the amphiphilic polymers are of straight-chain molecular structures, with one terminal being a hydrophilic molecular terminal and the other terminal being a hydrophobic molecular terminal.

Optionally, the nanoparticle comprises surface single nanoparticle and hybrid nanoparticle;
the surface single nanoparticle comprises hydrophilic particle, hydrophobic particle, and inorganic nanoparticle;
the hybrid nanoparticle is a hybrid nanoparticle obtained by modifying the surface single nanoparticle with a hybrid substance;
in the above, the hybrid substance is a modified membrane; and the modified membrane comprises cell membrane, exosome, oil membrane, hydrogel, and liposome; and
optionally, the hybrid nanoparticle is a particle formed by coating an outer surface of the surface single nanoparticle with the modified membrane, so that the hybrid nanoparticle modified from the surface single nanoparticle can be linked to arms of the hydrophilic polymers, arms of the hydrophobic polymers, or the POI recognition groups,

The nanoparticle has a particle size of 5-1000 nm.

Optionally, in the third degradation tool, the nanoparticle is hydrophobic particle, hydrophilic particle, or the surface single nanoparticle is coated with the modified membrane, and when the linkers are the amphiphilic polymers, the hydrophilic polymers or the hydrophobic polymers, methods of linking the linkers to the nanoparticle comprise: non-covalently bonding the linkers to the nanoparticle, or covalently bonding the linkers to the nanoparticle through reactive groups modified on the linkers.

Optionally, the antibodies in the POI recognition groups are therapeutic monoclonal antibodies, multispecific antibodies, nanobodies or the preceding antibodies' derivatives or antibody-drug conjugates;
the peptides are peptides having specific POI binding capacity; and
the small molecules are small molecule compounds having specific POI binding capacity.

In addition, in order to solve the abovementioned problems, the present disclosure further provides use of the abovementioned nano-structural protein degradation tool in preparation of drugs, vaccines, and delivery carriers for treatment and prevention of abnormal protein accumulation diseases, wherein the abnormal protein accumulation diseases include tumors, immune system diseases, inflammations and pathogen infections, neurodegenerative diseases, blood system diseases, and metabolic diseases.

In addition, in order to solve the abovementioned problems, the present disclosure further provides a method for treatment and prevention of abnormal protein accumulation diseases, including administering to a subject a therapeutically effective amount of the nano-structural protein degradation tool, wherein the abnormal protein accumulation diseases include tumors, immune system diseases, inflammations and pathogen infections, neurodegenerative diseases, blood system diseases, and metabolic diseases.

In addition, in order to solve the abovementioned problems, the present disclosure further provides use of the abovementioned nano-structural protein degradation tool in preparation of a detection product and/or kit for abnormal protein accumulation diseases, wherein the abnormal protein accumulation diseases include tumors, immune system diseases, inflammations and pathogen infections, neurodegenerative diseases, blood system diseases, and metabolic diseases.

In addition, in order to solve the abovementioned problems, the present disclosure further provides a preparation method of the abovementioned nano protein degradation tool, including:
preparation of the first degradation tool and the second degradation tool, including: non-covalently bonding the POI recognition groups to the nanoparticle or linkers, or covalently bonding the POI recognition groups to the nanoparticle or linkers through active groups, thus forming the nano-structural protein degradation tool;
preparation of the third degradation tool, including:
firstly coupling the POI recognition groups with the linkers to form coupling intermediates; and then linking the coupling intermediates to the nanoparticle at the core, thus forming the third degradation tool; or
firstly constructing nanocomposite structures with the linkers as an outer layer and the nanoparticle as a core; and then coupling the POI recognition groups with the nanocomposite structure of the nanoparticle, thus forming the nano protein degradation tool.

The present disclosure provides a nano-structural protein degradation tool, use, and a preparation method thereof, wherein the nano-structural protein degradation tool includes: one of or a combination of several of a first degradation tool, a second degradation tool, and a third degradation tool, wherein the first degradation tool is formed by linking POI recognition groups to linkers; the second degradation tool is formed by linking the POI recognition groups to nanoparticle; the third degradation tool is formed by linking the POI recognition groups to the nanoparticle through the linkers; and the POI recognition groups include antibodies, proteins, peptides, nucleic acid aptamers or small molecules capable of specifically binding to the POI.

The present disclosure further provides a lipid-based protein degradation tool, including:
POI recognition groups, and lipid hybrid substance linked to the POI recognition groups, wherein
the POI recognition groups include antibodies, proteins, peptides, nucleic acid aptamers or small molecules capable of specifically binding to the POI; and
the lipid hybrid substance comprises liposome, exosome, cell membrane and LNP.

Optionally, when the POI recognition groups are coupled with the lipid hybrid substance, the lipid-based protein degradation tool is nanoparticle composed of the lipid hybrid substance at a core and the POI recognition groups located at periphery for protein degradation.

Optionally, the lipid-based protein degradation tool further comprises the lipid-based protein degradation tool provided with linking members between the POI recognition groups and the lipid hybrid substance;
optionally, the linking members have a molecular weight of 0-1000 kDa, not including 0 kDa;
the linking members are one of polymer linkers and lipid linkers;
optionally, the polymer linkers include hydrophilic polymers, hydrophobic polymers, and amphiphilic polymers; and
optionally, the lipid linkers are amphiphilic lipid linkers.

Optionally, when the POI recognition groups are coupled with the lipid hybrid substance through the linking members, the POI recognition group and the linking member constitute a set of linking unit; the lipid-based protein degradation tool is the lipid-based protein degradation tool having a multi-layer structure and composed of the lipid hybrid substance located at the core and a plurality of sets of linking units, wherein the linking unit comprises the linker located at an intermediate layer and linked to the lipid hybrid substance, and the POI recognition group located at periphery and linked to the linking member.

Optionally, the lipid linker includes at least two terminals, one terminal being a lipophilic terminal capable of being linked to the lipid hybrid substance, and the other terminal being a hydrophilic terminal; and
optionally, the lipophilic terminal is a lipid molecule.

Optionally, the amphiphilic polymers are polymers of chain-like or branched molecular structures, which have at least one hydrophilic molecular terminal and one hydrophobic molecular terminal; and
optionally, the amphiphilic polymers are of straight-chain molecular structures, with one terminal being a hydrophilic molecular terminal and the other terminal being a hydrophobic molecular terminal.

Optionally, nanoparticle is further comprised, wherein the nanoparticle is coated by the lipid hybrid substance at the core of the lipid-based protein degradation tool;
the nanoparticle comprises hydrophilic particle, hydrophobic particle, and inorganic nanoparticle; and
optionally, the nanoparticle has a particle size of 5-1000 nm.

In addition, in order to solve the abovementioned problems, the present disclosure further provides use of the abovementioned lipid-based protein degradation tool in preparation of drugs and delivery systems for treatment and prevention of abnormal protein accumulation diseases, wherein the abnormal protein accumulation diseases include tumors, immune system diseases, neurodegenerative diseases, blood system diseases, and metabolic diseases.

In addition, in order to solve the abovementioned problems, the present disclosure further provides a method for treatment and prevention of abnormal protein accumulation diseases, including administering to a subject a therapeutically effective amount of the lipid-based protein degradation tool, wherein the abnormal protein accumulation diseases include tumors, immune system diseases, inflammations and pathogen infections, neurodegenerative diseases, blood system diseases, and metabolic diseases.

In addition, in order to solve the abovementioned problems, the present disclosure further provides use of the abovementioned lipid-based protein degradation tool in preparation of a detection product and/or kit for abnormal protein accumulation diseases, wherein the abnormal protein accumulation diseases include tumors, immune system diseases, neurodegenerative diseases, blood system diseases, and metabolic diseases.

In addition, in order to solve the abovementioned problems, the present disclosure further provides a preparation method of the abovementioned lipid-based protein degradation tool. When the lipid-based protein degradation tool is a protein degradation tool formed by coupling the POI recognition groups with the lipid hybrid substance, the preparation method thereof is: non-covalently bonding the POI recognition groups to the lipid hybrid substance; or covalently bonding the POI recognition groups to the lipid hybrid substances through coupling groups, so as to form the lipid-based protein degradation tool.

Optionally, the lipid-based protein degradation tool further includes a protein degradation tool formed by linking the POI recognition groups and the lipid hybrid substances through linking members;
in the above, when the lipid-based protein degradation tool is the protein degradation tool formed by linking the POI recognition groups and the lipid hybrid substances through the linking members, the preparation method thereof is:
firstly coupling the POI recognition groups with the linking members to form coupling intermediates; and then linking the coupling intermediates to the lipid hybrid substances, thus forming the lipid-based protein degradation tool; or
firstly constructing nanocomposite structures with the linking members as an outer layer and the lipid hybrid substances as a core; and then coupling the POI recognition groups with the nanocomposite structures, thus forming the lipid-based protein degradation tool.

The present disclosure provides the lipid-based protein degradation tool, use, and the preparation method thereof, wherein the lipid-based protein degradation tool includes: POI recognition groups, and lipid hybrid substances linked to the POI recognition groups, wherein the POI recognition groups include antibodies, proteins, polypeptides, nucleic acid aptamers or small molecules capable of specifically binding to the POI; and the lipid hybrid substances include liposomes, exosomes, cell membranes and LNP.

### BRIEF DESCRIPTION OF DRAWINGS

In order to more clearly illustrate technical solutions of embodiments of the present disclosure, accompanying drawings which need to be used in the embodiments will be introduced briefly below, and it should be understood that the accompanying drawings below merely show some embodiments of the present disclosure, and therefore should not be considered as limitation to the scope, those ordinarily skilled in the art still could obtain other relevant drawings according to these accompanying drawings, without using creative efforts.

FIG. 1 is the structural schematic diagram of the first degradation tool of the nano protein degradation tool of the present disclosure;

FIG. 2 is the structural schematic diagram of the second degradation tool of the nano protein degradation tool of the present disclosure;

FIG. 3 is the structural schematic diagram of the third degradation tool of the nano protein degradation tool of the present disclosure;

FIG. 4 is the schematic diagram of synthesis of the first degradation tool in Example 1 of the present disclosure;

FIG. 5 shows the Coomassie brilliant blue staining result of the first degradation tool in Example 1 of the present disclosure;

FIG. 6 shows the protein electrophoresis result of protein degradation effect of the first degradation tool in Example 1 of the present disclosure;

FIG. 7 is the schematic diagram of synthesis of the second degradation tool in Example 2 of the present disclosure;

FIG. 8 shows the structure of JQ1-NH₂ used in JQ1-NP nanoparticles of the second degradation tool in Example 2 of the present disclosure and the dynamic light scattering result of the JQ1-NP nanoparticles;

FIG. 9 shows the protein electrophoresis result of JQ1-NP of the second degradation tool in Example 2 of the present disclosure;

FIG. 10 shows the protein electrophoresis result of the second degradation tool NTZ-NP2 in Example 2 of the present disclosure;

FIG. 11 is the schematic diagram of synthesis of the third degradation tool in Example 3 of the present disclosure;

FIG. 12 is the schematic diagram of structural morphology of the third degradation tool in Example 3 of the present disclosure;

FIG. 13 shows results of dynamic light scattering and transmission electron microscopy of the third degradation tool in Example 3 of the present disclosure;

FIG. 14 shows protein electrophoresis results of protein degradation effects of the third degradation tool (NTZ-NP) in Example 3 of the present disclosure in human breast cancer M231 cells, human cervical cancer HeLa cells, and human brain glioma U87 cells;

FIG. 15 shows immunofluorescence staining results of M231 cells after being treated with the third degradation tool;

FIG. 16 shows EGFR protein degradation results of green-fluorescence-labeled living cells chronologically photographed in a fixed visual field after M231 cells are treated by the third degradation tool;

FIG. 17 shows cell viability results of HepG2 cells after being treated with the third degradation tool tested by CCK8 kit;

FIG. 18 shows EGFR protein expressions of the third degradation tool in Example 3 of the present disclosure, wherein a is for effective degradation concentration exploration; and b is for protein recovery exploration after treatment;

FIG. 19 shows EGFR protein expressions of the third degradation tool in Example 3 of the present disclosure, exploring influence of different ratios between the coupling intermediate composed of POI recognition groups and linkers and the blank linker in the absence of the POI recognition groups on degradation effects;

FIG. 20 is the structural schematic diagram of the third degradation tool (NTZ-AuNP) in Example 4 of the present disclosure;

FIG. 21 shows an electrophoresis result of protein degradation effect of the third degradation tool (NTZ-AuNP) in Example 4 of the present disclosure;

FIG. 22 shows the proton nuclear magnetic resonance spectrum (¹H NMR) of coupling product (ACE2-PEG-DSPE) of POI recognition groups and linkers in the third degradation tool (ACE2-NP) in Example 5 of the present disclosure;

FIG. 23 shows the proton nuclear magnetic resonance spectrum (¹H NMR) of coupling product (CD13-PEG-DSPE) of POI recognition groups and linkers in the third degradation tool (CD13-NP) in Example 5 of the present disclosure;

FIG. 24 is the diagram of detection result of GFP by flow cytometry for ACE2-GFP labeled 293T cells, which are treated by the third degradation tool (ACE2-NP) in Example 5 of the present disclosure;

FIG. 25 shows the protein electrophoresis result of human hepatoma cell HepG2 after being treated with the third degradation tool (CD13-NP) in Example 5 of the present disclosure;

FIG. 26 shows an experimental result of endocytosis of FITC green dye-labeled β-amyloid 1-42 oligomer detected by confocal microscope after human glial cells are treated with the third degradation tool (AB-NP) in Example 5 of the present disclosure;

FIG. 27 shows the proton nuclear magnetic resonance spectrum (¹H NMR) of coupling product (Palb-PEG-DSPE) of POI recognition groups and linkers in the third degradation tool (Palb-NP) in Example 6 of the present disclosure;

FIG. 28 shows the protein electrophoretogram result of protein degradation effect of the third degradation tool (Palb-NP) in Example 6 of the present disclosure;

FIG. 29 shows experimental results of crystal violet staining clone formation after M231 cells are treated with the third degradation tool in Example 6 of the present disclosure;

FIG. 30 is the structural schematic diagram of the third degradation tool (AV45-NP) in Example 6 of the present disclosure;

FIG. 31 shows the proton nuclear magnetic resonance spectrum (¹H NMR) of coupling product (AV45-PEG-DSPE) of POI recognition groups and linkers in the third degradation tool (AV45-NP) in Example 6 of the present disclosure;

FIG. 32 shows an experimental result of endocytosis of FITC green dye-labeled β-amyloid 1-42 oligomer detected by confocal microscope after human glial cells are treated with the third degradation tool (AB-NP) in Example 6 of the present disclosure;

FIG. 33 shows the protein electrophoretogram result of protein degradation effect of the third degradation tool (CTX-NP) in Example 7 of the present disclosure;

FIG. 34 is the confocal microscope image of the protein degradation effect of the third degradation tool (PTZ-NP) in Example 7 of the present disclosure;

FIG. 35 shows the protein electrophoretogram result of protein degradation effect of the third degradation tool (ATZ-NP) in Example 7 of the present disclosure;

FIG. 36 shows the protein electrophoretogram result of protein degradation effect of the third degradation tool (CRLZ-NP) in Example 7 of the present disclosure;

FIG. 37 shows the protein electrophoretogram result of protein degradation effect of the third degradation tool (INE/NTZ-NP) in Example 7 of the present disclosure;

FIG. 38 and FIG. 39 show the results of in vivo animal experiments of the third degradation tool (NTZ-NP) in Example 8 of the present disclosure, wherein FIG. 38a shows the tumor volumes of nude mice during NTZ-NP treatment on M231 cells in nude mouse subcutaneous tumor models;

FIG. 38b shows the EGFR expression of dissected and isolated tumors after the NTZ-NP treatment detected by protein blotting electrophoresis along with the statistical results; and FIG. 38c shows change in body weights of nude mice during the NTZ-NP treatment;

FIG. 39 shows results of EGFR detected by immunofluorescent staining for isolated tumor tissues after the NTZ-NP treatment is completed;

FIG. 40 shows results of apoptotic markers detected by immunofluorescent staining for isolated tumor tissues after the NTZ-NP treatment is completed;

FIG. 41 shows detection of blood parameters for liver function and kidney function of mice after a single administration of the third degradation tool (NTZ-NP) in Example 8 of the present disclosure;

FIG. 42 shows the capacity of obtaining the blood-brain barrier crossing and tumor targeting ability by simple self-assembling of the third degradation tool (NTZ-NP) in Example 8 of the present disclosure, which can be traced due to the loading of fluorescent dye DIR, reflected by the brain accumulation of DIR fluorescence detected by a small animal imager and statistical chart;

FIG. 43 shows diagrams of results of immunohistochemical detection of EGFR and cell proliferation marker PCNA after brain tumor dissection after the third degradation tool (NTZ-NP) of Example 8 is administered to in situ glioma-bearing animal models by the assembling method in FIG. 42;

FIG. 44 is the structural schematic diagram of coupling the POI recognition groups with the lipid hybrid substances;

FIG. 45 is the structural schematic diagram of coupling the POI recognition groups with the lipid hybrid substances containing the linking members;

FIG. 46 is the structural schematic diagram of linking the POI recognition groups and the lipid hybrid substances containing the linking members-lipid linkers;

FIG. 47 is the structural schematic diagram of NTZ-PEGlipo synthesis in Example 9 of the present disclosure;

FIG. 48 shows the protein electrophoresis result for the degradation effect of NTZ-PEGlipo on target protein EGFR in M231 cells in Example 9 of the present disclosure;

FIG. 49 shows schematic diagrams of synthesis of NTZ-lipo1, NTZ-lipo2, INE-lipo, Palb-lipo, and AV45-lipo in Example 10 of the present disclosure;

FIG. 50 shows protein electrophoresis results for the degradation effect of NTZ-lipo1 at different concentrations on target protein EGFR in M231 cells in Example 10 of the present disclosure;

FIG. 51 shows protein electrophoresis results for the degradation effect on target protein EGFR of NTZ-lipo1 with different composition ratios of lipid hybrid substances in M231 cells in Example 10 of the present disclosure;

FIG. 52 shows protein electrophoresis results for the degradation effect on target protein EGFR of NTZ-lipo2 in M231 cells in Example 10 of the present disclosure;

FIG. 53 shows protein electrophoresis results for the degradation effect on target protein HER2 of INE-lipo in M231 cells in Example 10 of the present disclosure;

FIG. 54 shows the result of proton nuclear magnetic resonance spectrum (¹H NMR) of the product after coupling of the lipid linkers with the POI recognition groups used by Palb-lipo in Example 10 of the present disclosure;

FIG. 55 shows the result of proton nuclear magnetic resonance spectrum (¹H NMR) of the product after coupling of the lipid linkers with the POI recognition groups used by AV45-lipo in Example 10 of the present disclosure;

FIG. 56 shows protein electrophoresis results of degradation effect on target protein CDK4 of Palb-lipo at different concentrations in M231 cells in Example 10 of the present disclosure;

FIG. 57 is the structural schematic diagram of AV45-lipo in Example 10 of the present disclosure;

FIG. 58 shows confocal microscope images of labeled cellular lysosomal, fluorescent dye FITC-labeled AP, and AV45 autofluorescence, wherein the detection by confocal microscope was performed after co-incubation of AV45-lipo and β-amyloid (Aβ) oligomer in human glial cell culture medium followed by PBS washing in Example 10 of the present disclosure;

FIG. 59 is the structural schematic diagram of NTZ-LNP1, NTZ-LNP1s, and NTZ-LNP2 in Example 11 of the present disclosure;

FIG. 60 shows particle size distribution results of dynamic light scattering (DLS) of NTZ-LNP1, NTZ-LNP1s, and NTZ-LNP2 in Example 11 of the present disclosure;

FIG. 61 shows protein electrophoresis results of degradation effect on target protein EGFR of NTZ-LNP1 in M231 cells in Example 11 and NTZ-lipo1 in Example 10;

FIG. 62 shows protein electrophoresis results of degradation effect on target protein EGFR of NTZ-LNP1s in M231 cells in Example 11 of the present disclosure;

FIG. 63 shows protein electrophoresis results of degradation effect on target protein EGFR of NTZ-NTZ-LNP2 in M231 cells in Example 11 of the present disclosure;

FIG. 64 is the PDI diagram of DLS particle size distribution and average dispersion coefficient of NTZ-exo in Example 12 of the present disclosure;

FIG. 65 shows protein electrophoresis results of degradation effect on target protein EGFR of NTZ-exo in M231 cells in Example 12 of the present disclosure;

FIG. 66 is the structural schematic diagram of NTZ-lipoP in Example 13 of the present disclosure;

FIG. 67 shows protein electrophoresis results of degradation effect on target protein EGFR of NTZ-lipoP at different concentrations in M231 cells in Example 13 of the present disclosure;

FIG. 68 is the structural schematic diagram of CTX-RBCmD in Example 13 of the present disclosure;

FIG. 69 is the diagram of DLS particle size distribution results of CTX-RBCmD in Example 13 of the present disclosure; and

FIG. 70 shows protein electrophoresis results of degradation effect on target protein EGFR of CTX-RBCmD in M231 cells in Example 13 of the present disclosure.

### REFERENCE SIGNS

100. lipid-based protein degradation tool; 1. POI recognition group; 2. liposome, exosome, or cell membrane in lipid hybrid substance; 3. LNP in lipid hybrid substance; 4. lipid linker; 5. polymer linker; 6. coupling group; 7. nanoparticle.

Implementation of the objectives, functional features, and advantages of the present disclosure are further described with reference to the accompanying drawings in combination with examples.

### DETAILED DESCRIPTION OF EMBODIMENTS

Technical solutions of the present disclosure will be described clearly and completely below in connection with examples, and apparently, the examples described are only a part of examples of the present disclosure, rather than all examples. All of other examples obtained by those ordinarily skilled in the art based on the examples in the present disclosure without using creative efforts shall fall within the scope of protection of the present disclosure.

Unless otherwise defined in the following text, all technical terms and scientific terms used in the embodiments of the present disclosure have the same meanings as those generally understood by those ordinarily skilled in the art. Although the following terms are believed to be well understood by those skilled in the art, the following definitions are set forth to better explain the present disclosure.

As used herein, the terms “include”, “comprise”, “have”, “contain”, or “involve” are inclusive or open-ended and do not exclude other unlisted elements or method steps. The term “consist of . . . ” is to be regarded as a preferred embodiment of the term “comprise”. If a certain group is defined below as comprising at least a certain number of embodiments, it should also be understood as disclosing a group that is preferably composed only of these embodiments.

An indefinite or definite article used when referring to nouns in the singular form, such as “one” or “a”, “the”, the plural form of the nouns is included.

The term “about” in the present disclosure indicates an accuracy interval that can be understood by those skilled in the art and guarantee the technical effects of the features discussed. This term generally means±10%, preferably, ±5% of deviation from an indicated value.

In addition, the terms first, second, third, (a), (b), (c), and the like in the description and the claims are used for distinguishing similar elements and not necessarily for describing a sequential or chronological order. It should be understood that the terms so applied are interchangeable under appropriate circumstances and that the embodiments described herein may be implemented in other sequences than described or exemplified in the present disclosure.

The following are provided merely to help understanding the present disclosure. These definitions should not be construed as having a scope smaller than that understood by those skilled in the art.

The technical solutions of the present disclosure will be further described in detail below in conjunction with specific examples, but are not intended to limit the present disclosure in any way, and any limited modifications made by any person within the scope of the claims of the present disclosure are still within the scope of the claims of the present disclosure.

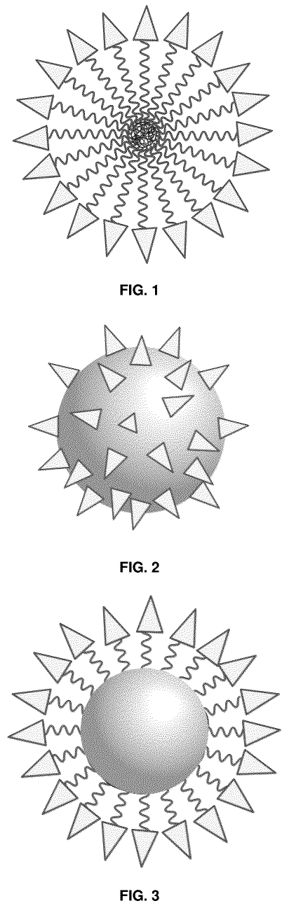

The present disclosure provides a nano-structural protein degradation tool, including:
one of or a combination of several of a first degradation tool (refer to FIG. 1 for a structure thereof), a second degradation tool (refer to FIG. 2 for a structure thereof), and a third degradation tool (refer to FIG. 3 for a structure thereof);
in the above, the first degradation tool is formed by linking POI recognition groups to linkers; the second degradation tool is formed by linking the POI recognition groups to nanoparticle; and the third degradation tool is formed by linking the POI recognition groups to the nanoparticle through the linkers; and
the POI recognition groups include antibodies, proteins, peptides, nucleic acid aptamers or small molecules capable of specifically binding to the POI.

The POI recognition groups are antibodies, proteins, peptides, nucleic acid aptamers or small molecules capable of specifically binding to the POI.

The nanoparticle is nano particulate or nano granule, and may be of a solid granular structure, a hollow structure or a porous granular structure. In the granular structure, the composition may be a single component, and also may be a multi-component composite. In the above, the nanoparticle may be biocompatible nanoparticle.

The linkers are structures and substances which can promote improving the water solubility and stability, and have the function of linking POI recognition groups, or bridging the POI recognition groups and the nanoparticle, so as to make them form an integral structure.

The nano-structural protein degradation tool includes three manifestation forms (structural morphologies), i.e. the first degradation tool, the second degradation tool, and the third degradation tool. Specific structural morphologies are illustrated by the following table:

TABLE 1
Three Structural Morphologies of the Nano-structural
Protein Degradation Tool (TPD-NP)

| | Component | | | | |
|---|---|---|---|---|---|
| | | POI | | | |
| | | recognition | | | Final |
| No. | TPD-NP | group | Linker | Nanoparticle | product structure |
| 1 | First | √ | √ | x | POI recognition |
| | degradation | | | | group + linker |
| | tool | | | | |
| 2 | Second | √ | x | √ | POI recognition |
| | degradation | | | | group + |
| | tool | | | | nanoparticle |
| 3 | Third | √ | √ | √ | POI recognition |
| | degradation | | | | group + linker + |
| | tool | | | | nanoparticle |

The POI recognition groups include: antibodies, proteins, peptides, nucleic acid aptamers or small molecules capable of specifically binding to the POI.

In the above, the antibodies may include therapeutic monoclonal antibodies, multispecific antibodies, nanobodies or derivatives or antibody-drug conjugates of the preceding antibodies;
the proteins may be: proteins having a specific POI binding capacity;
the peptides are peptides having a specific POI binding capacity; and include binding peptides of ACE2, CD13, β-Amyloid;
the nucleic acid aptamers are nucleic acid aptamers having a specific POI binding capacity;
the small molecules are small molecule compounds having a specific POI binding capacity, including derivatives of CDK4/6 protein inhibitor Palbociclib, derivatives of BRD4 protein inhibitor JQ1, derivatives of β-Amyloid protein probe AV-45, derivatives of Pittsburgh compound PiB, and derivatives of Tau protein probe GTP1 and PBB3. The antibodies include: CTX, NTZ, PTZ, CRLZ, INE, ATZ, AND, and MTX. In the above, CTX (Cetuximab) is the most classical human-mouse chimeric EGFR protein monoclonal antibody, and has been approved by more than 100 countries/regions around the world, for treating wild type RAS metastatic colorectal cancer, and locally advanced and recurrent and metastatic head and neck squamous cell carcinoma. NTZ (Nimotuzumab) is the most classical EGFR protein monoclonal antibody in China, for treating various kinds of tumors caused by EGFR mutation. PTZ (pertuzumab) is a HER2-targeted monoclonal antibody drug researched and developed by Roche and marketed in 2012, and has a binding site different from that of trastuzumab to HER2 protein. CRLZ (Camrelizumab) PD-1 inhibitor is a classical immunotherapeutical monoclonal antibody for the treatment of lung cancer, liver cancer, esophagus cancer, and Hodgkin's lymphoma. INE (Inetetamab) is a HER2 monoclonal antibody, and is currently used for the treatment of HER2 positive breast cancer. ATZ (Atezolizumab) is a PD-L1 humanized monoclonal antibody, and is a classical tumor immunotherapy target. Adunatumab ADN (aducanumab) is a monoclonal antibody for Alzheimer's disease and can bind to β-amyloid protein. MTX (Miltuximab) is a monoclonal antibody of anti-glypican 1, and is an emerging tumor treatment target.

In the present disclosure, by modifying the POI recognition groups on surface of the nanoparticle, or linking the POI recognition groups to the linkers, or modifying the POI recognition groups on the linkers and linking the POI recognition groups to the surface of the nanoparticle through the linkers, after assembly, the POI recognition groups are exposed to the outside of the nanoparticle or the linkers, wherein the nanoparticle can permeate cells without depending on specially designed receptor-ligand matching pairs, and can be coupled with the POI recognition groups such as small molecules, peptides, nucleic acid aptamers, and antibodies, and specifically perform protein hijacking and targeted degradation, so as to realize the assembly of the TPD tool (TPD-NP) based on the nanoparticles (NP). Such targeted nano-structural protein degradation tool with simple drug carrying and tissue specific targeting capacity enables drug and protein degradation tool synergy treatment and translational/precision medicine to become possible. The invention of TPD-NP and exploration of mechanism thereof greatly expand the range of the TPD tool, provide basic knowledge for the TPD and the field of nano delivery, and in principle, can degrade extracellular/membrane-related/intracellular proteins related to a variety of human diseases in vivo.

Optionally, in the nano-structural protein degradation tool, after construction, the three degradation tools as a whole have spatial structural morphologies as shown in the following table:

TABLE 2
Three Spatial Structural Morphologies of Nano-structural
Protein Degradation Tool (TPD-NP)

| No. | TPD-NP | Structural morphology |
|---|---|---|
| 1 | First | The POI recognition group and the linker |
| | degradation | constitute a set of linking unit; and the first |
| | tool | degradation tool is the nano-structural protein |
| | | degradation tool having a multi-layer structure |
| | | and composed of a plurality of sets of linking |
| | | units, wherein the linking unit comprises the |
| | | linker located at a core and the POI recognition |
| | | group linked to the linker and located at periphery |
| 2 | Second | the second degradation tool is the nano-structural |
| | degradation | protein degradation tool having a multi-layer |
| | tool | structure and composed of the nanoparticle |
| | | located at the core and a plurality of the POI |
| | | recognition groups linked to the |
| | | nanoparticle and located at periphery |
| 3 | Third | The POI recognition groups are linked to the |
| | degradation | nanoparticle through the linkers; and the |
| | tool | third degradation tool is the nano-structural |
| | | protein degradation tool having a multi-layer |
| | | structure and composed of the nanoparticle |
| | | located at the core and a plurality of sets of |
| | | linking units, wherein the linking unit comprises |
| | | the linker linked to the nanoparticle |
| | | and located at an intermediate layer, |
| | | and the POI recognition group |
| | | located at periphery and linked to the linker |

The nano-structural protein degradation tool has different spatial structures of TPD-NP formed by different linking manners.

In the first degradation tool, since the POI recognition group and the linker constitute a set of linking unit, the first degradation tool as a whole is of a multi-layer structure with the interior being a core and the exterior being periphery, a plurality of linking units are located in the multi-layer structure, and morphology thereof is that one terminal of the linker is at the core and the POI recognition group linked to the linker is at the periphery, thus constituting the TPD-NP in a granular form with a multi-layer structure from inside to outside.

In the above, in the second degradation tool, since the POI recognition groups are linked to the nanoparticle, the spatial structural morphology of the second degradation tool is also formed by a multi-layer mechanism, with the interior being a core and the exterior being periphery, the inner core is single nanoparticle, and the plurality of POI recognition groups linked thereto are located at the periphery, so as to form the second degradation tool in a granular form with an inner and outer double-layer structure.

In the above, in the third degradation tool, since the link mode thereof is that the POI recognition groups are linked to the nanoparticle through the linkers, i.e., “POI recognition groups-linkers-nanoparticle”. In the third degradation tool, the whole is a multi-layer structure, and is divided into three layers, wherein the interior is a core, the middle part is an intermediate layer, and the exterior is periphery. Based on the linking mode, the inner core is a nanoparticle, then a plurality of linkers linked to the nanoparticle are in the intermediate layer, and the POI recognition groups linked to the linkers are at the periphery, thus forming the third degradation tool in the granular form with a three-layer structure.

Optionally, the linkers include hydrophilic polymers, hydrophobic polymers, and amphiphilic polymers;

According to the water-soluble property thereof, the linkers can be divided into three different kinds of linkers, including: 1. hydrophilic polymers, 2. hydrophobic polymers, 3. amphiphilic polymers.

The hydrophilic polymers may include, but are not limited to, polyethylene glycol (PEG), polyethylene oxide (PEO), poly(ethylene glycol) methacrylate (POEG), poly 2-methacryloyloxyethyl phosphorylcholine (PMPC), polycarboxybetaine (PCB), dextran, hyaluronic acid, chitosan, β-cyclodextrin, hyperbranched polyglycidyl ether (HPG), poly N-(2-hydroxypropyl)methacrylamide (PHPMA), polyhydroxyethyl methacrylate (PHEMA), polyacrylamide (PAM), polyvinylpyrrolidone (PVP), polyacrylic acid (PAA), polymaleic anhydride (HPMA), polyquaternary ammonium salts and pharmaceutically acceptable polymeric salts thereof, polyethyleneimine (PEI), poly(N,N-dimethylaminoethyl methacrylate (PDMAEMA), and various hydrophilic polyamino acids, such as polylysine (PLL), polyglutamic acid (PGu), and polyaspartic acid (PAsp), and derivatives of the abovementioned polymers and pharmaceutically acceptable salts thereof.

The hydrophobic polymers may include, but are not limited to, polylactic acid-glycolic acid (PLGA) copolymers, polylactic acid (PLA), polycaprolactone (PCL), polycarbonate (PMC) and derivatives thereof, copolymers of various combinations and components of glycolide/lactide/caprolactone/carbonate, polyurethane (PU), polyetheretherketone (PEEK), polymethyl methacrylate (PMMA), polyvinyl alcohol (PVA), polyethylene (PE), and various hydrophilic polyamino acids, such as polyphenylalanine, and derivatives of the abovementioned polymers and pharmaceutically acceptable salts thereof.

The amphipathic polymers may include, but are not limited to, PEG-PLGA, PEG-PCL, PEG-PLA, PEG-PMC, and various amphiphilic block polymers formed by the combination of the abovementioned hydrophilic polymers and hydrophobic polymers and derivatives, and various kinds of amphiphilic polymers composed of the abovementioned hydrophilic polymers and hydrophobic molecules (such as lipids) and derivatives. The abovementioned lipid molecules may include, but are not limited to, fatty acids, glycerolipids, glycerophospholipids, sphingolipids, sterol lipids, prenol lipids, saccharolipids, polyketides, cationic lipids, and ionizable lipids, and derivatives thereof.

Optionally, the linkers have a molecular weight of 0-1000 kDa, not including 0 kDa;
optionally, the amphipathic polymers include: amphipathic block co-polymers, amphipathic polymers composed of the hydrophilic polymers and hydrophobic small molecules, and amphipathic polymers composed of the hydrophobic polymers and hydrophilic small molecules.

The amphipathic polymers include three kinds, and structural forms thereof are respectively shown in the following table:

TABLE 3
Three Structural Forms of Amphiphilic Polymer

| | Name |
|---|---|
| No. | Amphiphilic polymer |
| 1 | Amphiphilic block co-polymer |
| 2 | Hydrophilic polymer + hydrophobic small molecule |
| 3 | Hydrophobic polymer + hydrophilic small molecule |

In the above, the amphiphilic block polymer is linear or branched, has two or more structurally different chain segments in a single polymer molecule, and can synthesize a copolymer having a specific chemical structure and molecular weight according to needs. The block polymer with amphipathy can self-assemble in a solution into a specific supermolecular ordered aggregate (micelle or vesicle). The amphiphilic block co-polymer, after dissolving in water, can spontaneously form a polymer micelle composed of a hydrophilic outer shell and a lipophilic inner core, or a polymer vesicle composed of a hydrophilic outer shell, a lipophilic intermediate layer, and a hydrophilic cavity. The other two are amphipathic polymers composed of the hydrophilic polymers and hydrophobic small molecules, and amphipathic polymers composed of the hydrophobic polymers and hydrophilic small molecules. In a preferred technical solution, the amphipathic polymers are polymers of chain-like or branched molecular structures, which at least have one hydrophilic molecular terminal and one hydrophobic molecular terminal.

The amphipathic polymers are polymers having a chain-like molecular structure, and may be polymers having a molecular structure formed by linking a plurality of linear chains, the molecular structure thereof has at least one hydrophilic molecular terminal and one hydrophobic molecular terminal, so that the amphipathic polymer has amphipathy.
optionally, the amphiphilic polymers are of a linear molecular structure, with one terminal being a hydrophilic molecular terminal and the other terminal being a hydrophobic molecular terminal.

In the above, the amphiphilic polymers have a linear structure, and the linear molecular structure includes two molecular terminals, wherein one of the molecular terminals is a hydrophilic molecular terminal, and the other terminal is a hydrophobic molecular terminal.

It should be noted that, as the linkers may function to perform water solubility modification on the nanoparticles or to improve steric hindrance of the POI recognition groups, when the water solubility of the nanoparticles need to be increased and the steric hindrance of the POI recognition groups need to be improved, the nanoparticles need to be linked by the linkers.

If the nanoparticles are lipid-soluble polymers, water-soluble polymers or inorganic nanoparticles in organic polymer nanoparticles, it is necessary to use the linkers in the nano-structural protein degradation tool to enhance the stability of the nanoparticles and bridge the nanoparticles and the POI recognition groups.

When the nanoparticles are amphiphilic polymers, the nanoparticles have amphipathy of both hydrophilicity and hydrophobicity, and thus there is no need to enhance or improve the hydrophilicity thereof, in this case, the nanoparticles are directly linked to the POI recognition groups without using the function of the linkers.

Optionally, the nanoparticles include surface single nanoparticles and hybrid nanoparticles;
the surface single nanoparticles include hydrophilic particles, hydrophobic particles, and inorganic nanoparticles.

The inorganic nanoparticles include gold nanoparticles, carbon nanoparticles, silicon nanoparticles, iron oxide nanoparticles, calcium phosphate nanoparticles, barium sulphate and iodide contrast agents, aluminium nitride nanoparticles, aluminium oxide nanoparticles, titanium oxide nanoparticles, aluminium-iron alloy particles, and titanium-iron alloy particles, all of which are materials having stability and low biotoxicity.

The surface single nanoparticles may be biocompatible materials. Optionally, the surface single nanoparticles are biocompatible materials.

The hydrophilic particles may be dendritic polymers, hyperbranched polymers, various nanogels composed of the preceding hydrophilic polymers and derivatives thereof, and nano albumins.

The hydrophobic particles may be nanoparticles prepared by various kinds of polymeric structure such as polylactic acid-glycolic acid (PLGA) copolymers, polylactic acid (PLA), polycaprolactone (PCL), polycarbonate (PMC) and derivatives thereof, copolymers of glycolide/lactide/caprolactone/carbonate, polyurethane (PU), polyetheretherketone (PEEK), polymethyl methacrylate (PMMA), polyvinyl alcohol (PVA), polystyrene (PS), polyethylene (PE), polyphenylalanine hydrophobic polyamino acids and derivatives thereof. The hybrid nanoparticles are hybrid nanoparticles obtained by modifying the surface single nanoparticles with a hybrid substance.

The hybrid substance may be a modified membrane. The nanoparticles are enabled to have a certain change in water solubility by being coated by the modified membrane. The modified membrane may include a cell membrane, an exosome, an oil membrane, a hydrogel, and a liposome.

Optionally, the hybrid nanoparticles are particles formed by coating an outer surface of the surface single nanoparticles with the modified membrane, so that the hybrid nanoparticles modified from the surface single nanoparticle can be linked to the hydrophilic polymers, arms of the hydrophobic polymers, arms of the amphiphilic polymers, or the POI recognition groups.

The nanoparticles have a particle size of 5-1000 nm;

In the third degradation tool, the nanoparticle is hydrophobic particle, hydrophilic particle, or the surface single nanoparticle is coated with the modified membrane. When the linkers are the amphiphilic polymers, the hydrophilic polymers or the hydrophobic polymers, methods of linking the linkers to the nanoparticle comprise: non-covalently bonding the linkers to the nanoparticle, or covalently bonding the linkers to the nanoparticle through reactive groups modified on the linkers;
optionally, antibodies in the POI recognition groups are therapeutic monoclonal antibodies, multispecific antibodies, nanobodies or derivatives or antibody-drug conjugates of the preceding antibodies;
the peptides are peptides having a specific POI binding capacity; and including binding polypeptides of ACE2, CD13, β-Amyloid;
the nucleic acid aptamers are nucleic acid aptamers having a specific POI binding capacity; and
the small molecules are small molecule compounds having a specific POI binding capacity, including derivatives of CDK4/6 protein inhibitor Palbociclib, derivatives of BRD4 protein inhibitor JQ1, derivatives of β-Amyloid protein probe AV-45, derivatives of Pittsburgh compound PiB, and derivatives of Tau protein probe GTP1 and PBB3.

The antibodies include: CTX, NTZ, PTZ, CRLZ, INE, ATZ, AND, and MTX. In the above, CTX (Cetuximab) is the most classical human-mouse chimeric EGFR protein monoclonal antibody, and has been approved by more than 100 countries/regions around the world, for treating RAS wild type metastatic colorectal cancer, and locally advanced and recurrent and metastatic head and neck squamous cell carcinoma. NTZ (Nimotuzumab) is the most classical EGFR protein monoclonal antibody in China, for treating various kinds of tumors caused by EGFR mutation. PTZ (pertuzumab) is a HER2-targeted monoclonal antibody drug researched and developed by Roche and marketed in 2012, and has a binding site different from that of trastuzumab to HER2 protein. CRLZ (Camrelizumab) PD-1 inhibitor is a classical immunotherapeutical monoclonal antibody for the treatment of lung cancer, liver cancer, esophagus cancer, and Hodgkin's lymphoma. INE (Inetetamab) is HER2 monoclonal antibody, and is currently used for the treatment of HER2 positive breast cancer. ATZ (Atezolizumab) is a PD-L1 humanized monoclonal antibody, and is a classical tumor immunotherapy target. Adunatumab ADN (aducanumab) is a monoclonal antibody for Alzheimer's disease and can bind to β-amyloid protein. MTX (Miltuximab) is a monoclonal antibody of anti-glypican 1, and is an emerging tumor treatment target.

In addition, the present disclosure further provides use of the abovementioned nano-structural protein degradation tool in preparation of drugs, vaccines, and delivery carriers for treatment and prevention of abnormal protein accumulation diseases, wherein the abnormal protein accumulation diseases include tumors, immune system diseases, neurodegenerative diseases, blood system diseases, and metabolic diseases.

In addition, the present disclosure further provides use of the abovementioned nano-structural protein degradation tool in preparation of a detection product and/or kit for abnormal protein accumulation diseases, wherein the abnormal protein accumulation diseases include tumors, immune system diseases, inflammations and pathogen infections, neurodegenerative diseases, blood system diseases, and metabolic diseases.

In the present disclosure, such protein degradation tools are used for a variety of pathology-relevant protein degradation, and in situ tumor pathology-relevant protein degradation is tested in animal tumor models.

The use range includes, but is not limited to, drug development and diagnosis for diseases involving abnormal protein accumulation, such as tumors, immune system diseases, inflammations and pathogen infections, neurodegenerative diseases, blood system diseases, and metabolic related diseases, development of protein degradation tool in in vitro detection and biomedical research, and kit development in protein interaction research.

In addition, the present disclosure further provides a preparation method of a nano protein degradation tool, including:
(1) preparation method 1 for the first or second degradation tool: non-covalently bonding the POI recognition groups to the nanoparticles or linkers;
(2) preparation method 2 for the first or second degradation tool: covalently bonding the POI recognition groups to the nanoparticles or linkers based on active groups, thus forming the nano protein degradation tool;
(3) preparation method 1 for the third degradation tool: firstly coupling the POI recognition groups with the linkers to form coupling intermediates; and then linking the coupling intermediates to the particles at the core, thus forming the third degradation tool;
(4) preparation method 2 for the third degradation tool: firstly constructing nanocomposite structures with the linkers as an outer layer and the nanoparticles as a core; and then coupling the POI recognition groups with the composite structure of the nanoparticles, thus forming the nano-structural protein degradation tool.

In the above, when preparing, constructing or assembling the nano-structural protein degradation tool, it may include, but is not limited to, the abovementioned three manners, and corresponding methods may be selected for the first, second, and third degradation tools to prepare corresponding protein degradation tools.

In the above, in (1) and (2), there are two methods for preparing the first or second degradation tool, and they can be specifically selected according to specific properties of nanoparticles, in (3) and (4), there are two methods of preparing the third degradation tool, and they are different in the linking sequences, wherein in (3) the POI recognition groups are firstly coupled with the linkers, and then linked to the core portions of the particles, and in (4) the linkers are firstly linked to the nanoparticles to form composite structures, and then the composite structures are coupled with the POI recognition groups.

Referring to FIG. 44, the present disclosure further provides a lipid-based protein degradation tool, including:

POI recognition groups, and lipid hybrid substances linked to the POI recognition groups, wherein the POI recognition groups include antibodies, proteins, peptides, nucleic acid aptamers or small molecules capable of specifically binding to the POI; and the lipid hybrid substances include liposomes, exosomes, cell membranes and LNP (lipid nanoparticle).

The POI recognition groups are antibodies, proteins, peptides or small molecules capable of specifically binding to the POI.

The lipid hybrid substances include liposome, exosome, cell membrane and LNP. The LNP is lipid nanoparticle.

It should be noted that, lipid nanoparticles are mainly used for in vivo drug delivery, from traditional liposomes to lipid nanoparticles (LNP), they have been widely used in the fields such as small molecule drug delivery, nucleic acid drug delivery, and nanovaccine. The mRNA vaccines of COVID-19 mostly consist of LNP.

The lipid nanoparticles have the advantages of low costs and easy preparation, and the composition rule thereof is also well studied. The lipid nanoparticles used for medical and pharmaceutical development have a set of relatively regular components, with the most basic components being membrane skeleton and cholesterol excipients. The lipid nanoparticles in clinical use also can be added with other components as required, which may include, but are not limited to, the following components:
1. membrane skeleton, a main component constituting the lipid double-layer membrane;
2. auxiliary excipients: (1) cholesterol, adjusting the fluidity of the membrane and improving the particle stability, and (2) auxiliary phospholipid, maintaining micro-morphology of the liposome and making the lysosome membrane unstable;
3. PEGylated lipid, reducing the binding of particles to plasma proteins in vivo, and prolonging systemic circulation time;
4. cationic lipid, efficiently entrapping nucleic acid drugs, providing positive charges, transfecting in vivo, and pH-sensitive (ionizable type); and stabilizing agent, having a freeze-drying protection function and maintaining the structural stability of the liposome in the freeze-drying process.

The lipid hybrid substances include liposomes, exosomes, cell membranes, LNP, LPP, and lipid nanoemulsion. In the above, the liposome is also referred to as classical liposome, of which the morphology is vacuolated, and which is mainly used for carrying hydrophobic drugs. As the liposome serves as an amphiphilic membrane, the hydrophobic vacuole can carry hydrophobic drugs, and the hydrophilic vacuoles can carry hydrophilic drugs. However, LNP is a lipid nanoparticle, and contains a cationic lipid therein. Currently, mRNA vaccines and some nucleic acid vaccines use LNP. As nucleic acids are negatively charged, cationic lipids are positively charged, the entrapping efficiency can be effectively improved, and LNP is also used for delivery of CRISPR gene editing elements. However, the conventional liposome lipo has a low nucleic acid carrying efficiency, and generally carries hydrophobic and hydrophilic small molecule drugs. As containing a polymer core, the LPP obtains the characteristics of lipids and polymers, and has better drug carrying compatibility and stability.

In addition, LNP and LPP also have the advantages of being easily modified and reconstructed. By means of the amphiphilic PEGylated lipid and similar structure, it is extremely easy to modify the liposome and LNP. The PEGylated lipid can be linked to ligands through PEG terminal for binding to receptors, which may facilitate delivery of drugs to a target organ, and is also referred to as actively targeted lipid nanocarrier.

The lipid hybrid substances facilitate drug carrying. The drugs include biotechnological drugs, such as hydrophilic and hydrophobic small molecule drugs, nucleic acid drugs, protein drugs, and gene editing carriers, all of which can be effectively carried and delivered. However, the use thereof is still focused on drug delivery, physiological and pathological properties of the structure itself still need to be further explored, and more therapeutic potentials and possibilities are worth exploring.

In conclusion, in the present disclosure, by modifying the POI recognition groups on the surfaces of the lipid hybrid substances, and after assembly, exposing the POI recognition groups to the outside of the lipid hybrid substances, the target protein degradation based on the lipid hybrid substances is realized, so that the synthesis difficulty of constructing the protein degradation tool is greatly reduced. By means of the “plug and play” mode, massive compounds, peptides, antibodies, nucleic acid aptamers, etc. having a binding capacity to the target protein can be upgraded to be protein degradation drugs, to further play a new role in the fields related to conventional liposome and lipid nanoparticle (LNP), such as mRNA vaccines, nucleic acid delivery carriers, and drug delivery, thus realizing combined therapy development in scientific research and industrial applications.

In addition, the search for the use of ligand-targeted lipid nanoparticles (lipid hybrid substances) as protein degradation tool and the mechanism thereof is still blank. The present disclosure greatly extends the current use range of lipid nanoparticles, provides basic knowledge for the fields of TPD and nano delivery, and can, in principle, degrade in vivo extracellular/membrane-related/intracellular proteins associated with various human diseases.

Optionally, when the POI recognition groups are coupled with the lipid hybrid substances, the lipid-based protein degradation tool is nanoparticles composed of the lipid hybrid substances at a core and the POI recognition groups located at periphery for protein degradation.

In the structure of the abovementioned lipid-based protein degradation tool, the whole may be a granulated structure, including the core and the periphery, wherein the core part may be a lipid hybrid substance, and the periphery is a plurality of POI recognition groups linked to the lipid hybrid substance, i.e. there is a layer of numerous POI recognition groups linked to the lipid hybrid substance on an outer surface of the lipid hybrid substance, thus forming the whole lipid-based protein degradation tool.

In addition, depending on the morphology of the lipid hybrid substance, in principle, if it is a classical liposome, its morphology is vacuolated.

Optionally, referring to FIG. 45, the lipid-based protein degradation tool further may include the lipid-based protein degradation tool provided with linking members between the POI recognition groups and the lipid hybrid substances;

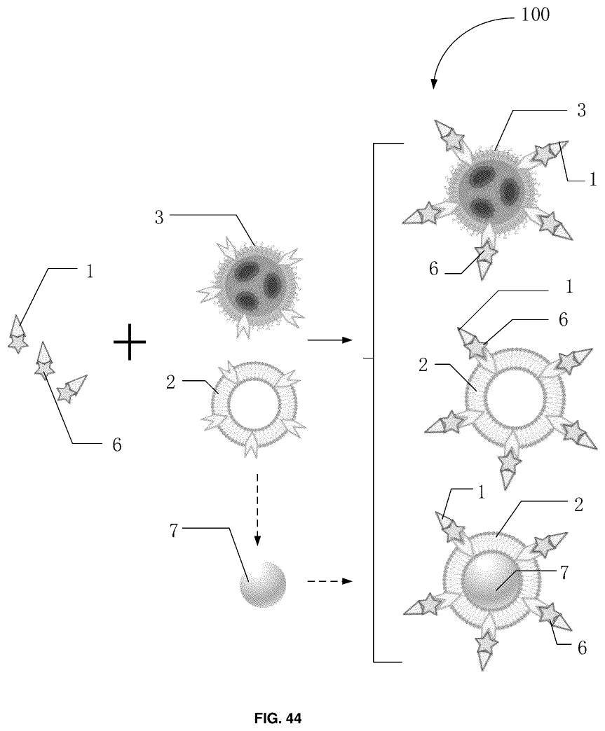

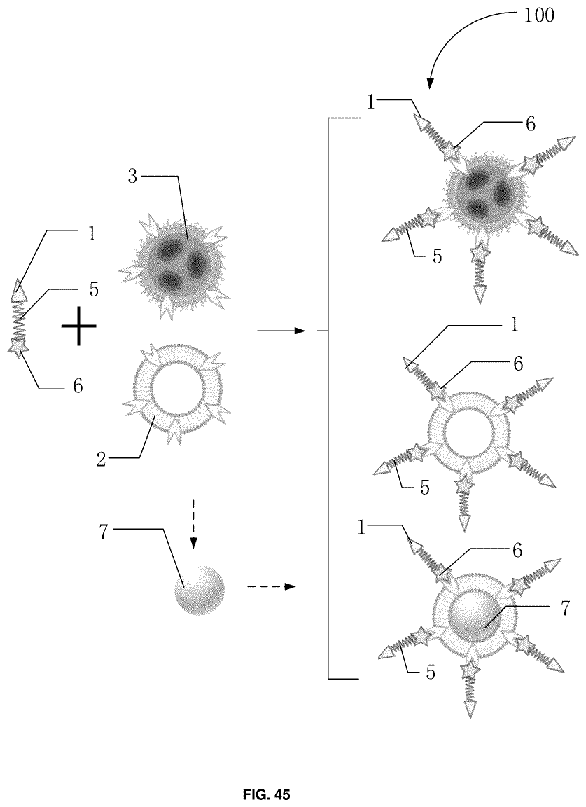

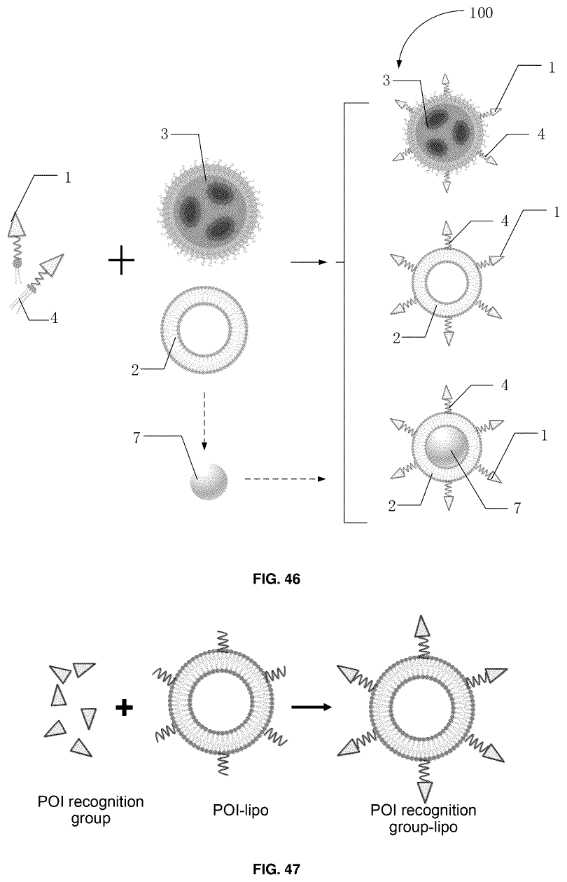

Optionally, the linking members have a molecular weight of 0-1000 kDa, not including 0 kDa; and
the linking members are one of polymer linkers and lipid linkers, wherein the lipid-based protein degradation tool containing the lipid linkers has a structure as shown in FIG. 46.

Optionally, the polymer linkers include hydrophilic polymers, hydrophobic polymers, and amphiphilic polymers.

Optionally, the lipid linkers are amphiphilic lipid linkers.

The present disclosure provides another structure of the lipid-based protein degradation tool. Similar to the basic structure, a linking member is further provided between the lipid hybrid substance and the POI recognition group. A combination of sequential linking of “lipid hybrid substance-linking member-POI recognition group” is constituted, wherein the linking member is provided with a coupling group capable of linking with the POI recognition group, so that the combination of the two can be achieved.

Optionally, when the POI recognition groups are coupled with the lipid hybrid substance through the linking members, the POI recognition group and the linking member constitute a set of linking unit; the lipid-based protein degradation tool is the lipid-based protein degradation tool having a multi-layer structure and composed of the lipid hybrid substance located at the core and a plurality of sets of linking units, wherein the linking unit comprises the linker located at an intermediate layer and linked to the lipid hybrid substance, and the POI recognition group located at periphery and linked to the linking member.

In this case, for a spatial structure of the lipid-based protein degradation tool, the core is a lipid hybrid substance (core), a plurality of linking members are linked to a surface layer of the lipid hybrid substance (intermediate layers), and each linking member is linked to a lipid hybrid substance (periphery), thus forming a granular structure having three layers from inside to outside. The structure as a whole may be spherical in principle, and may also be in other shapes.

Optionally, the linking members have a molecular weight of 0-1000 kDa, not including 0 kDa.

The linking members are one of polymer linkers and lipid linkers.

Optionally, the polymer linkers include hydrophilic polymers, hydrophobic polymers, and amphiphilic polymers.

TABLE 4
Three Structural Morphologies of Polymer Linker

| | Name |
|---|---|
| No. | Polymer Linker |
| 1 | Hydrophilic polymer |
| 2 | Hydrophobic polymer |
| 3 | Amphiphilic polymer |

Optionally, the lipid linkers are amphiphilic lipid linkers.

According to the characteristics of the linking members, the linking members are divided into two major categories, including: polymer linkers and lipid linkers, any of which can be selected according to synthesis requirements and water solubility requirements of substances, and the lipid linkers are amphiphilic lipid linkers.

The hydrophilic polymers may include, but are not limited to, polyethylene glycol (PEG), polyethylene oxide (PEO), poly(ethylene glycol) methacrylate (POEG), poly 2-methacryloyloxyethyl phosphorylcholine (PMPC), polycarboxybetaine (PCB), dextran, hyaluronic acid, chitosan, β-cyclodextrin, hyperbranched polyglycidyl ether (HPG), poly N-(2-hydroxypropyl)methacrylamide (PHPMA), polyhydroxyethyl methacrylate (PHEMA), polyacrylamide (PAM), polyvinylpyrrolidone (PVP), polyacrylic acid (PAA), polymaleic anhydride (HPMA), polyquaternary ammonium salts and pharmaceutically acceptable polymeric salts thereof, polyethyleneimine (PEI), poly(N,N-dimethylaminoethyl methacrylate (PDMAEMA), and various hydrophilic polyamino acids, such as polylysine (PLL), polyglutamic acid (PGu), and polyaspartic acid (PAsp), and derivatives of the abovementioned polymers and pharmaceutically acceptable salts thereof.

The hydrophobic polymers may include, but are not limited to, polylactic acid-glycolic acid (PLGA) copolymers, polylactic acid (PLA), polycaprolactone (PCL), polycarbonate (PMC) and derivatives thereof, copolymers of various combinations and components of glycolide/lactide/caprolactone/carbonate, polyurethane (PU), polyetheretherketone (PEEK), polymethyl methacrylate (PMMA), polyvinyl alcohol (PVA), polyethylene (PE), and various hydrophilic polyamino acids, such as polyphenylalanine, and derivatives of the abovementioned polymers and pharmaceutically acceptable salts thereof.

The amphipathic polymers may include, but are not limited to, PEG-PLGA, PEG-PCL, PEG-PLA, PEG-PMC, and various amphiphilic block polymers formed by the combination of the abovementioned hydrophilic polymers and hydrophobic polymers and derivatives, and various kinds of amphiphilic polymers composed of the abovementioned hydrophilic polymers and hydrophobic molecules (such as lipids) and derivatives.

Optionally, the lipid linker includes at least two terminals, one terminal being a lipophilic terminal capable of being linked to the lipid hybrid substance, and the other terminal being a hydrophilic terminal; and
optionally, the lipophilic terminal is a lipid molecule.

When the linking members are lipid linkers, the structure thereof may include a plurality of terminals, but at least two terminals thereof are the lipophilic terminal (linked to lipid hybrid substance) and the hydrophilic terminal, and the lipophilic terminal is a lipid molecule.

The lipid molecules include: fatty acids, glycerolipids, glycerophospholipids, sphingolipids, sterol lipids, prenol lipids, saccharolipids, polyketides, cationic lipids, and ionizable lipids.

Optionally, the lipid molecules may be DSPE (distearoyl phosphatidyl ethanolamine), distearoyl phosphatidylcholine (DSPC), 1,2-dimyristoyl-sn-glycerol (DMG), 1,2-dipalmitoyl-sn-glycerol (DPG), 1,2-diphytanoyl-sn-glycerol (DPyG), or diacylglycerol (DAG); triacylglycerol (TAG), 1,2-dip almitoyl-sn-glycerol (DPG), 1,1′-R1R)-1-(hydroxymethyl)-1,2-ethanediylloctadecanoate (DSG), diarachidoyl phosphatidylcholine (DAPC), 1,2-dilauroyl-sn-glycero-3-phosphocholine (DLPC), dimyristoyl phosphatidylcholine (DMPC), 1,2-dioleoyl phosphatidylcholine (DOPC), dipalmitoylphosphatidylcholine (DPPC), 1,2-dipalmitoyl-sn-glycero-3-ethylphosphocholine (DPePC), phosphatidylcholine (PC), phosphatidylethanolamine (PE), phosphatidylglycerol (PG), phosphatidylserine (PS), phosphatidylinositol (PI); ceramide (Cer), sphingomyelin (SM), cholesterol (Cho), cholesterol ester (CE), 1,2-dimyristoyl-SN-glycero-3-phosphate (DMPA), dilauroyl phosphatidic acid (DLPA), phosphocholine, 1,2-dioleoyloxy-3-(trimethylammonium)propane (DOTAP), the ionizable lipids include DLin-MC3-DMA, A6, OF-02, A18-ISO5-2DC18, 98_{N12}-5, 9A1P, C12-200, cKK-E12, 7c1, GO-C14, L319, 304O₁₃, OF-Deg-Lin, 306-0B12, 306-Oᵢ₁₀, and FTT5, and derivatives of the preceding lipid molecules and pharmaceutically acceptable salts thereof. The PEG lipids may be PEG and conjugates of PEG derivatives and the preceding lipids.

Optionally, the lipid molecules may be PEGylated lipids, wherein the relative molecular weight of PEG is 2000, and the PEGylated lipids may include: PEG-DSPE (polyethylene glycol-distearoyl phosphatidyl ethanolamine), PEG-DMG (polyethylene glycol-dimyristic glyceride), conjugates of the preceding lipid molecules and PEG and pharmaceutically acceptable salts thereof. The PEG includes PEG and PEG with a PEG terminal being methoxy or other groups.

Optionally, the amphiphilic polymers are polymers of a chain-like or branched molecular structure, which at least have one hydrophilic molecular terminal and one hydrophobic molecular terminal; and the structure of the amphiphilic polymers is chain-like or branched molecular structure, and due to the characteristics of the chain-like or branched molecular structure thereof, they can have a plurality of branches and a plurality of chains, but they have at least two terminals, with one being a hydrophilic molecular terminal, and the other being a hydrophobic molecular terminal.

Optionally, the amphiphilic polymers are of a linear molecular structure, with one terminal being a hydrophilic molecular terminal and the other terminal being a hydrophobic molecular terminal.

In the above, the amphiphilic polymers are further defined as having a linear molecular structure.

Optionally, nanoparticles are further included, wherein the nanoparticles are coated by the lipid hybrid substances at the core of the lipid-based protein degradation tool;
the nanoparticles include hydrophilic particles, hydrophobic particles, and inorganic nanoparticles; and
optionally, the nanoparticles have a particle size of 5-1000 nm.

In another embodiment, a special structure is provided, i.e., the nanoparticle is at the core, the lipid hybrid substances wrap the nanoparticle to form a composite structure.

Thus, the lipid-based protein degradation tool may include several morphological structures as follows:

TABLE 5
Morphological Structures of Lipid-based Protein Degradation Tool

| | Structure | | |
|---|---|---|---|
| | | Intermediate | |
| No. | Core | layer | Periphery |
| 1 | Lipid hybrid substance | | POI recognition group |
| 2 | Lipid hybrid substance | Linking | POI recognition group |
| | | member | |
| 3 | Lipid hybrid substances | | POI recognition group |
| | coat the nanoparticle | | |
| 4 | Lipid hybrid substances | Linking | POI recognition group |
| | coat the nanoparticle | member | |

Besides, in another embodiment, a special structure is provided, i.e., the nanoparticle is at the core, the lipid hybrid substances coat the nanoparticle to form a composite structure.

In the above, the nanoparticle is coated by the lipid hybrid substances at the core of the lipid-based protein degradation tool;
the nanoparticle (the nanoparticle coated at the core by the lipid hybrid substances) includes:
(1) hydrophilic particles, which may be dendritic polymers, hyperbranched polymers, various nanogels composed of the preceding hydrophilic polymers and derivatives thereof, and nano albumins;
(2) hydrophobic particles, which may be nanoparticles prepared by polylactic acid-glycolic acid (PLGA) copolymers, polylactic acid (PLA), polycaprolactone (PCL), polycarbonate (PMC) and derivatives thereof, copolymers of various combinations and components of glycolide/lactide/caprolactone/carbonate, polyurethane (PU), polyetheretherketone (PEEK), polymethyl methacrylate (PMMA), polyvinyl alcohol (PVA), polyethylene (PE), and various hydrophilic polyamino acids, such as polyphenylalanine and derivatives thereof;
(3) the inorganic nanoparticles may be gold nanoparticles, carbon nanoparticles, silicon nanoparticles, iron oxide nanoparticles, calcium phosphate nanoparticles, barium sulphate and iodide contrast agents, aluminium nitride nanoparticles, aluminium oxide nanoparticles, titanium oxide nanoparticles, aluminium-iron alloy particles, and titanium-iron alloy particles, all of which are materials having stable and low biotoxicity; and (4) mixed nanoparticles formed by the preceding nanoparticles.

In the above, when the nanoparticle is one of the hydrophilic particle or the hydrophobic particle, and it is a polymer, after being coated by the lipid hybrid substance, it can also be referred to as a lipopolyplex (LPP), that is, a lipid membrane is coated on the surface of the nanoparticle of a polymer having hydrophilic or hydrophobic property, and emulsion droplet composed of cationic nanoemulsion, i.e. cationic lipid, is also widely studied and applied in vaccine and drug delivery.

Optionally, in the POI recognition groups, the antibodies are therapeutic monoclonal antibodies, multispecific antibodies, nanobodies or derivatives or antibody-drug conjugates of the preceding antibodies;
the peptides are peptides having a specific POI binding capacity; and
the small molecules are small molecule compounds having a specific POI binding capacity.
optionally, the small molecules include derivatives of CDK4/6 protein inhibitor Palbociclib, derivatives of BRD4 protein inhibitor JQ1, derivatives of β-Amyloid protein probe AV-45, derivatives of Pittsburgh compound PiB, and derivatives of Tau protein probe GTP1 and PBB3.

Optionally, the polypeptides include binding polypeptides of ACE2, CD13, β-Amyloid;
optionally, the antibodies include: CTX, NTZ, PTZ, CRLZ, INE, ATZ, and aducanumab, Miltuximab.

In addition, the present disclosure further provides use of the lipid-based protein degradation tool in preparation of drugs, vaccines, and delivery systems for treatment and prevention of diseases related to abnormal protein accumulation, wherein the abnormal protein accumulation diseases include tumors, immune system diseases, inflammations and pathogen infections, neurodegenerative diseases, blood system diseases, and metabolic diseases.

In addition, the present disclosure further provides use of the lipid-based protein degradation tool in preparation of a detection product and/or kit for diseases related to abnormal protein accumulation, wherein the abnormal protein accumulation diseases include tumors, immune system diseases, inflammations and pathogen infections, neurodegenerative diseases, blood system diseases, and metabolic diseases.

In addition, the present disclosure further provides a preparation method of a lipid-based protein degradation tool, wherein when the lipid-based protein degradation tool is a protein degradation tool formed by linking the POI recognition groups to the lipid hybrid substances, the preparation method thereof includes two methods:
(1) non-covalently bonding the POI recognition groups to the lipid hybrid substances; or
(2) covalently bonding the POI recognition groups to the lipid hybrid substances based on coupling groups, thus constituting the lipid-based protein degradation tool.

Optionally, the lipid-based protein degradation tool further includes a protein degradation tool formed by linking the POI recognition groups to the lipid hybrid substances through linking members;
in the above, when the lipid-based protein degradation tool is the protein degradation tool formed by linking the POI recognition groups and the lipid hybrid substances through the linking members, the preparation method thereof is:
(1) firstly coupling the POI recognition groups with the linking members to form coupling intermediates; and then linking the coupling intermediates to the lipid hybrid substances, thus forming the lipid-based protein degradation tool; or
(2) firstly constructing nanocomposite structures with the linking members as an outer layer and the lipid hybrid substances as a core; and then coupling the POI recognition groups with the nanocomposite structures, thus forming the lipid-based protein degradation tool.

In the present disclosure, by modifying the POI recognition groups on surfaces of the nanoparticles, or linking the POI recognition groups to the linkers, or modifying the POI recognition groups on the linkers and linking the POI recognition groups to the surfaces of the nanoparticles through the linkers, after assembly, the POI recognition groups are exposed to the outside of the nanoparticles or the linkers, wherein the nanoparticles (NP) can permeate cells without depending on specially designed receptor-ligand matching pairs, and can be coupled with the POI recognition groups such as small molecules, peptides, nucleic acid aptamers, and antibodies, and specifically perform protein hijacking and targeted degradation, so as to realize the assembly of the TPD tool (TPD-NP) based on the nanoparticles. Such convenient nanoparticle-based TPD tool is extremely easy to obtain drug carrying and tissue-specific targeting capacity, and enables drug and protein degradation combined treatment and transformation/precision medicine to become possible. The invention of TPD-NP and exploration of mechanism thereof greatly expand the range of the TPD tool, provide basic knowledge for the TPD and the field of nano delivery, and in principle, can degrade extracellular/membrane-related/intracellular proteins related to a variety of human diseases in vivo.

The present disclosure systematically proposes, for the first time, nanoparticle-mediated protein degradation, providing a new path for TPD and nano delivery. The nano-structural protein degradation tool of the present disclosure has a flexible structure, is convenient to modify, and can obtain the capabilities of drug carrying, targeting, and crossing biological barriers. The nano-structural protein degradation tool of the present disclosure has universality, a target can be randomly changed, all of the three components can be replaced, and the use scenarios are wide. All of the components of the nano-structural protein degradation tool of the present disclosure can be clinically approved materials, have a high potential for in vivo use, and have a conversion value. As a ready-to-use platform, the nano-structural protein degradation tool of the present disclosure does not require de novo synthesis from the raw chemical materials, thus greatly reducing the complexity and difficulty of development and production. Compared with that the PROTAC degrades intracellular proteins, and LYTAC degrades extracellular/membrane proteins, the TPD-NP can degrade extracellular/intracellular/membrane proteins. In addition, with respect to extracellular/membrane protein degradation tools such as LYTAC, the TPD-NP does not need to be additionally designed with a structure for assisting the hijacked proteins to be endocytosed. Compared with the existing TPD tools, the nano-structural protein degradation tool of the present disclosure does not need a special structure to guide protein degradation. Nanoparticles also can carry drugs, can be designed to be controllably released, can be designed to be photothermomagnetic and other synergistic treatment materials, and can be imaged and contrasted to further carry out synergistic treatment and integrated diagnosis and treatment of protein degradation. The humoral stability of NP can reduce drug loss and improve the pharmaceutical potential.

In addition, in the present disclosure, by modifying the POI recognition groups on the surfaces of the lipid hybrid substances, and after assembly, exposing the POI recognition groups to the outside of the lipid hybrid substances, the target protein degradation based on the lipid hybrid substances is realized, so that the synthesis difficulty of constructing the protein degradation tool is greatly reduced. By means of the “plug and play” mode, massive compounds, peptides, antibodies, nucleic acid aptamers, etc. having a binding capacity to the target protein can be upgraded to be protein degradation drugs, to further play a new role in the fields related to conventional liposome and lipid nanoparticle (LNP), such as mRNA vaccines, nucleic acid delivery carriers, and drug delivery, thus realizing combined therapy development in scientific research and industrial applications.

Moreover, the search for the use of ligand-targeted lipid nanoparticles (lipid hybrid substances) as protein degradation tool and the mechanism thereof is still blank. The present disclosure greatly extends the current use range of lipid nanoparticles, provides basic knowledge for the fields of TPD and nano delivery, and can, in principle, degrade extracellular/membrane-related/intracellular proteins associated with various human diseases in vivo.

The present disclosure is further described below with specific examples, but it should be understood that these examples are merely for more detailed description, but should not be construed as limiting the present disclosure in any form.

TABLE 6
Structural Morphologies of Protein
Degradation Tools Prepared in Examples 1-8

| | | POI | | | |
|---|---|---|---|---|---|
| | | recognition | | | |
| Example | | group | Linker | Nanoparticle | Name |
| 1 | | Nimotuzumab | PEG- | | |
| | | (NTZ) | DSPE | | |
| 2 | a | JQ1-NH₂ | | PEG-PLGA | JQ1-NP |
| | b | NTZ | | PEG-PLGA | NTZ-NP2 |
| | | antibody | | | |
| 3 | | NTZ | PEG- | PLGA | NTZ-NP |
| | | antibody | DSPE | | |
| 4 | | NTZ | PEG | Gold nanoparticle | NTZ-AuNP |
| | | antibody | | | |
| 5 | | Peptide | PEG- | PLGA | CD13-NP or |
| | | | DSPE | | ACE2-NP or |
| | | | | | AB-NP |
| 6 | | Small | PEG- | PLGA | Palb-NP or |
| | | molecule | DSPE | | AV45-NP |
| 7 | a | Cetuximab | PEG- | PLGA | CTX-NP |
| | | (CTX) | DSPE | | |
| | | antibody | | | |
| | b | Pertuzumab | PEG- | PLGA | PTZ-NP |
| | | (PTZ) | DSPE | | |
| | c | Atezolizumab | PEG- | PLGA | ATZ-NP |
| | | (ATZ) | DSPE | | |
| | d | Camrelizumab | PEG- | PLGA | CRLZ-NP |
| | | (CRLZ) | DSPE | | |
| | e | NTZ | PEG- | PLGA | NTZ/INE-NP |
| | | antibody and | DSPE | | |
| | | Inetetamab | | | |
| | | (INE) | | | |
| 8 | | NTZ | PEG- | PLGA | NTZ-NP |
| | | antibody | DSPE | | |

TABLE 7
Structural Morphologies of Protein Degradation Tools
Prepared in Examples 9-13

| | | POI | | | |
|---|---|---|---|---|---|
| | | recognition | Linking | | |
| Example | | group | member | Lipid hybrid substance | Name |
| 9 | | NTZ | None | PEG-Liposome | NTZ-PEGlipo |
| | | antibody | | (HSPC + cholesterol) | |
| 10 | a | NTZ | PEG- | Liposome | NTZ-lipo1 |
| | | antibody | DSPE | (HSPC + cholesterol) | |
| | b | NTZ | PEG- | Liposome | NTZ-lipo2 |
| | | antibody | DSPE | (DSPC + cholesterol) | |
| | c | Inetetamab | PEG- | Liposome | INE-lipo |
| | | (INE) | DSPE | (HSPC + cholesterol) | |
| | d | Small | PEG- | Liposome | Palb-lipoor |
| | | molecule | DSPE | (HSPC + cholesterol) | AV45-lipo |
| 11 | a | NTZ | PEG- | HSPC + cholesterol + | NTZ-LNP1 |
| | | antibody | DSPE | DOTAP | |
| | b | NTZ | PEG- | HSPC + cholesterol + | NTZ-LNP1s |
| | | antibody | DSPE | DOTAP + siRNA | |
| | c | NTZ | PEG- | HSPC + cholesterol + | NTZ-LNP2 |
| | | antibody | DMG | DOTAP | |
| 12 | NTZ antibody | | PEG- | exosome | NTZ-exo |
| | | | DSPE | | |
| 13 | a | NTZ | PEG- | Lipo membrane + coat | NTZ-lipoP |
| | | antibody | DSPE | PLGA nanoparticles | |
| | b | Cetuximab | PEG- | Erythrocyte membrane + | CTX-RBCmD |
| | | (CTX) | DSPE | coat glucan nanoparticles | |

### Example 1

Referring to Table 6, in the present example, a first degradation tool was prepared, including coupling POI recognition groups with linkers, and then self-assembling, wherein the POI recognition groups were monoclonal antibody drug nimotuzumab (NTZ), and the linkers were NHS-PEG-DSPE (N-hydroxysuccinimide modified polyethylene glycol-distearoyl phosphatidyl ethanolamine, or referred to as DSPE-PEG-NHS).

Preparation Method:

(1) pretreatment: replacing the buffer solution of original antibodies with a phosphate buffer solution PBS. In the present example, EGFR monoclonal antibody Nimotuzumab (NTZ) which has been applied to clinical treatment was used.

A nimotuzumab solution was centrifuged by a 3 kDa ultrafiltration tube at 4000×g for 2 minutes, then the antibodies were concentrated, and subsequently diluted with PBS; after concentration and dilution were repeated three times, main components of the buffer solution were replaced with PBS, after the antibody solution was diluted, protein concentration was determined in IgG mode by NanoDrop one C instrument according to the absorbance at A280, and the result thereof was verified by a BCA protein quantification kit.

(2) Coupling reaction: coupling reaction of the antibodies and the linkers was performed subsequently, and the antibodies were reacted with NHS-PEG (2 kDa)-DSPE through amino groups on the antibodies and NHS (N-hydroxysuccinimide) groups at DSPE-PEG terminals. The coupling reaction was started in an ice-water mixture environment, and was protected with nitrogen. In order to improve the dispersity, it can be selected to perform ultrasonic treatment on the DSPE-PEG in the PBS buffer solution at 40 kHz for 3 minutes, and then NHS-PEG-DSPE PBS solution of corresponding molar concentration and volume (the molar ratio is not limited to 1:1, referring to the general antibody-drug conjugate manufactory methods) was added to the antibody PBS solution stirred at 800 rpm (revolutions per minute); then the resultant was stirred and reacted for incubation on a 20 rpm rotator at 4° C. for 24 hours (in order to maintain antibody activity, it tends to be carried out at low speeds and 4° C.).

In the above, the coupling method includes, but is not limited to, non-site-fixed coupling and site-fixed coupling. The non-site-fixed coupling includes amino coupling, carboxyl coupling, bridging thiol coupling. The site-fixed coupling includes click reaction, selenium bond coupling, serine coupling, cysteine coupling, unnatural amino acid coupling, enzyme-catalyzed coupling, and sugar site coupling.

(3) Purification: reaction mixture was then subjected to ultrafiltration concentration by a kDa centrifuging ultrafiltration tube for 3 times, followed by dosing to 100 μL with PBS. Protein concentration was determined in IgG mode by NanoDrop one C instrument according to the absorbance at A280, and the result thereof was verified by a BCA protein quantification kit.

Experimental Results:

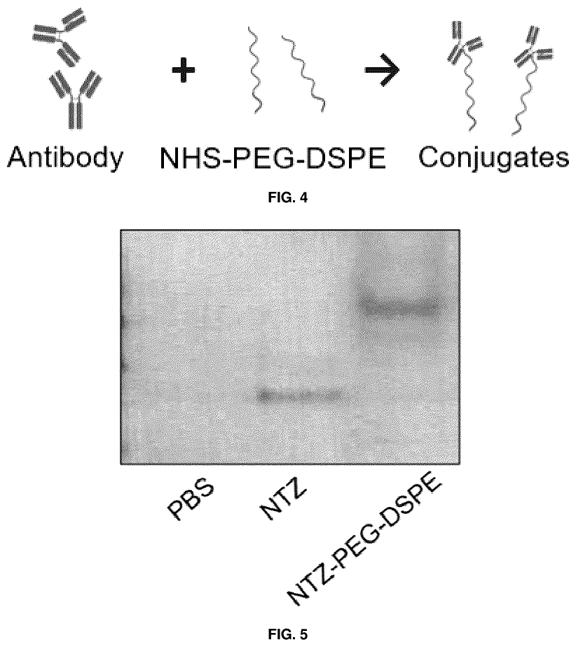

FIG. 4 of the present disclosure shows a synthesis process of the present example. In the above, the antibodies and NHS-PEG-DSPE were subjected to the coupling reaction through the amino groups on the antibodies and the NHS groups at the DSPE-PEG terminals, to obtain a conjugate NTZ-PEG-DSPE

FIG. 5 of the present disclosure shows a Coomassie brilliant blue staining result of the present disclosure. The Coomassie brilliant blue staining result shows that after reacting with the linkers to obtain the nanoparticle NTZ-PEG-DSPE, the NTZ antibodies have an increased mass, and a slowed migration rate, and a color developing band is on upper side after the Coomassie brilliant blue is combined with the protein, indicating that the POI recognition groups are successfully linked to the PEG-DSPE arms.

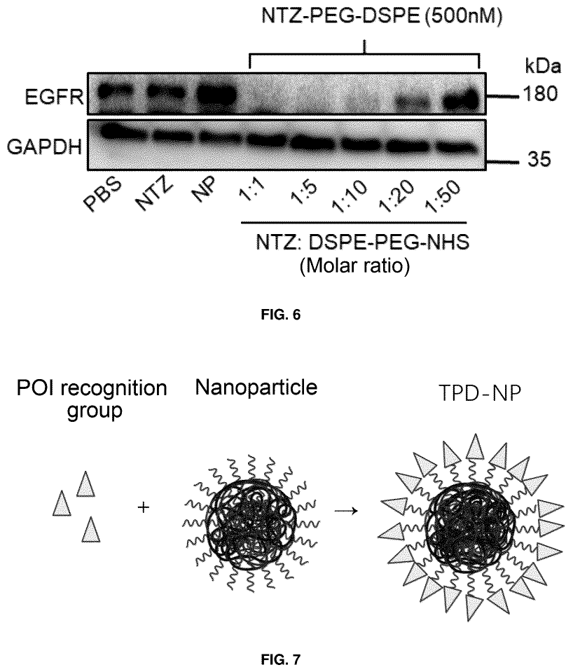

FIG. 6 of the present disclosure shows a Western blot protein electrophoretogram result of protein degradation effect of the present example. Result shows that the NTZ-PEG-DSPE nanoparticle can effectively degrade target protein EGFR.

It should be noted that cell culture media have the same volume in all the examples, and the unit of molar concentration of the treatment received thereby is μM (μmol/L) and nM (nmol/L). The molar concentrations used in all control groups are consistent with that used by the degradation tool groups, for example, the antibody NTZ in the NTZ group and the NTZ-NP group has the same molar concentration and the same volume, and meanwhile, the lipid hybrid substances in the NP group and the NTZ-NP group without the antibody have the same molar concentration and the same volume. In the above, the NP group refers to nanoparticles not linked with the POI recognition groups in the example. In all the examples involving multi-concentration test, the control group for POI recognition group and the control group for lipid hybrid substance were treated with the molar concentration equal to the highest concentration in concentration test group. All the protein electropherograms take GAPDH or VIN (vinculin) as internal reference.

### Example 2

Referring to Table 6, in the present example, the second degradation tool was prepared, including linking the POI recognition groups with the linkers, and subsequently self-assembling to form the second degradation tool. In a final product of the second degradation tool, the POI recognition groups were exposed to the outside of the whole. In the above, in Example 2a, the POI recognition groups are amino derivatives JQ1-NH₂ of inhibitor JQ1 of BRD4 protein, amino groups thereof are used for nanoparticle coupling, and BRD4 is a tumor epigenetic and proliferation regulatory molecule. In Example 2b, the POI recognition groups are EGFR monoclonal antibody Nimotuzumab NTZ, amino groups thereof are used for coupling of nanoparticles. In Example 2a and Example 2b, the nanoparticles are nanoparticles formed by transferring NHS-PEG-PLGA (N-hydroxy succinimide-polyethyleneglycol-polylactic acid-glycolic acid copolymer) from organic phase to aqueous phase, and the NHS is exposed to the outside of the nanoparticles.

In the above, the coupling method includes, but is not limited to, non-site-fixed coupling and site-fixed coupling. The non-site-fixed coupling includes amino coupling, carboxyl coupling, and bridging thiol coupling. The site-fixed coupling includes click reaction, selenium bond coupling, serine coupling, cysteine coupling, unnatural amino acid coupling, enzyme-catalyzed coupling, and sugar site coupling.

Experimental Method:

In the present Example 2a, an amino derivative JQ1-NH₂ of inhibitor JQ1 of BRD4 protein was selected as the POI recognition group, and was linked to a PEG terminal NHS group of PEG-PLGA nanoparticle through JQ1-NH₂.

The PEG-PLGA nanoparticles used were self-assembled into the nanoparticles by transferring polymer NHS-PEG (3 kDa)-PLGA (5 kDa) from organic phase to aqueous phase. Concentration was determined by the ultraviolet absorbance standard curve.

M231 (MDA-MB-231) human breast cancer tumor cells were treated for 24 hours with JQ1 with a final concentration of 500 nM, 1 μM, 1.5 μM, JQ1-NP with an equivalent molar amount of JQ1, and NP with an equivalent molar amount of JQ1, and subsequently total cellular proteins were harvested, to detect protein expression.

In the present Example 2b, NTZ monoclonal antibody was selected as the POI recognition group, and amino groups of NTZ-NTG monoclonal antibody lysine residues were coupled with PEG terminal NHS groups of the PEG-PLGA nanoparticles to obtain NTZ-NP2.

The PEG-PLGA nanoparticles used were self-assembled into nanoparticles by transferring NHS-PEG (3 kDa)-PLGA (5 kDa) from organic phase to aqueous phase. Concentration was determined in IgG mode by NanoDrop one C instrument according to the absorbance at A280, and the result thereof was verified by a BCA protein quantification kit.

Experimental Results:

FIG. 7 of the present disclosure is a schematic diagram of a synthesis process of the second degradation tool.

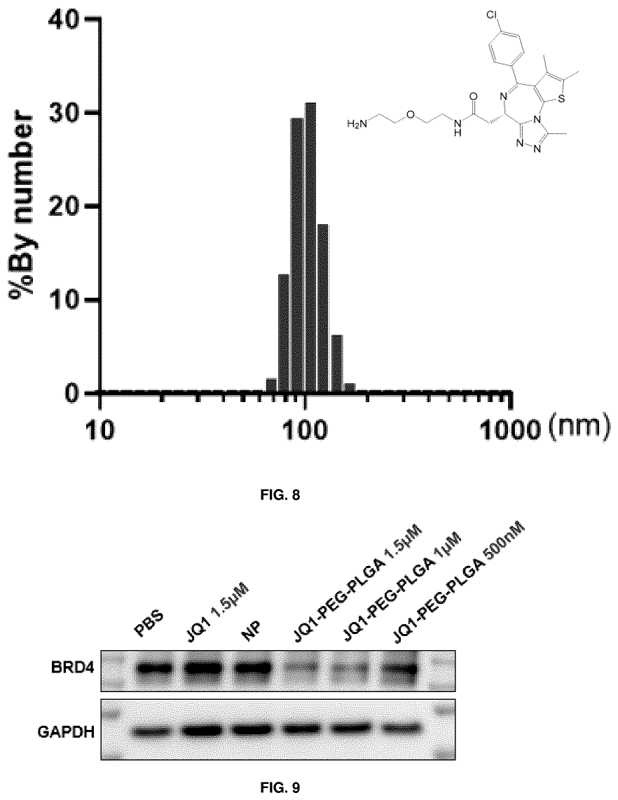

The structural formula in FIG. 8 of the present disclosure is the structure of JQ1-NH₂, and terminal free NH₂ thereof is used for coupling with PEG-PLGA nanoparticles. The dynamic light scattering DLS result in FIG. 8 shows that the nanoparticles have a particle size of about 100 nm. An average dispersion coefficient PDI (Polymer dispersity index) is about 0.2, indicating that the nanoparticles are relatively uniform.

FIG. 9 of the present disclosure shows a protein electrophoresis result of JQ1-NP of the second degradation tool in Example 2. The protein electrophoresis result shows that after JQ1 is coupled with the nanoparticles, cell BRD4 protein level is down-regulated, while none of solvent PBS control group, pure JQ1 small molecule group, and pure nanoparticle control group has the BRD4 degradation effect.

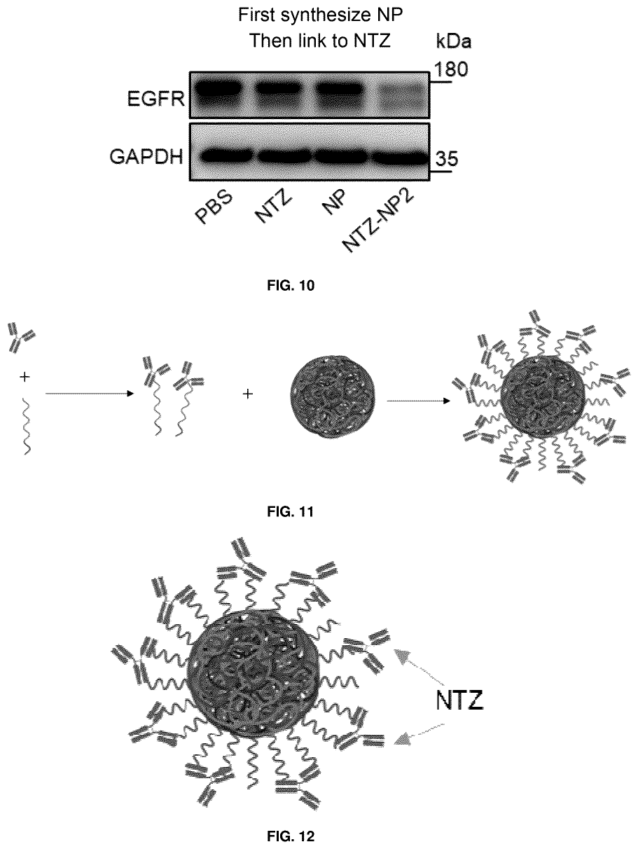

FIG. 10 of the present disclosure shows a protein electrophoresis result of the second degradation tool NTZ-NP2 in Example 2. The protein electrophoresis result shows that after 24 hours of treatment with 500 nM NTZ antibody-coupled nanoparticle NTZ-NP2, EGFR protein level of M231 cell is down-regulated, while none of the solvent PBS control group, pure EGFR monoclonal antibody group, and pure nanoparticle control group has the EGFR degradation effect.

### Example 3

Referring to Table 6, in the present example, a third degradation tool was prepared, including coupling POI recognition groups with linkers, then coupling the coupled POI recognition groups and linkers to the nanoparticles to form a composite structure. The POI recognition groups were NTZ antibodies. NTZ-PEG-DSPE coupling intermediates were obtained by coupling amino groups on the antibodies with NHS-PEG-DSPE linkers, and subsequently the NTZ-PEG-DSPE coupling intermediates were precipitated on the surfaces of the PLGA nanoparticles to generate a composite structure, thus finally forming the protein degradation tool, i.e. “NTZ-NP”, with the inner layer being nanoparticle core (PLGA), the intermediate layer being amphiphilic block polymers (PEG-DSPE), and the periphery being the NTZ antibodies.

1. Overall method of preparation: coupling of the POI recognition groups with the linkers PEG-DSPE employed the coupling method in the first degradation tool. The POI recognition group-PEG-DSPE was coupled with the PLGA nanoparticles, the PLGA was transferred from organic phase to aqueous phase, and the POI recognition group-PEG-DSPE was self-assembled with the PLGA in the aqueous phase and precipitated on the surface of the PLGA core.

Preparation Method:

(1) formulating a PBS solution of NTZ-PEG (2 kDa)-DSPE (molar ratio PLGA:NTZ-PEG-DSPE=1:1);

(2) dropwise adding the PLGA (15 kDa) dissolved in DMF (Dimethylformamide) (2 μL per drop, with an interval of 5 seconds for each drop) into a PBS solution of DSPE-PEG-NTZ stirred at 600 rpm, and the resultant was continuously stirred at room temperature for 30 minutes to complete self-assembly of PLGA and DSPE-PEG-NTZ;

(3) centrifuging and concentrating the solution 5 times by an ultrafiltration tube with molecular weight cutoff of 3 kDa, 4 minutes each time, to remove unbound components, and making up to a required volume with PBS; and

(4) determining protein concentration in IgG mode by NanoDrop one C (Thermofisher), and verifying protein concentration by a BCA protein assay kit.

### 2. Experiment 1: Verification of Substance Structure

FIG. 11 of the present disclosure shows a synthesis process of the third degradation tool. Coupling intermediates were obtained by coupling the amino groups on antibodies with linkers, and subsequently the coupling intermediates were precipitated on the surfaces of nanoparticles to generate a composite structure.

FIG. 12 of the present disclosure shows a morphological structure of the third degradation tool prepared in the present example, which is a protein degradation tool having an inner layer being a nanoparticle core (PLGA), an intermediate layer being an amphiphilic block co-polymer (PEG-DSPE), and a periphery being NTZ antibodies.

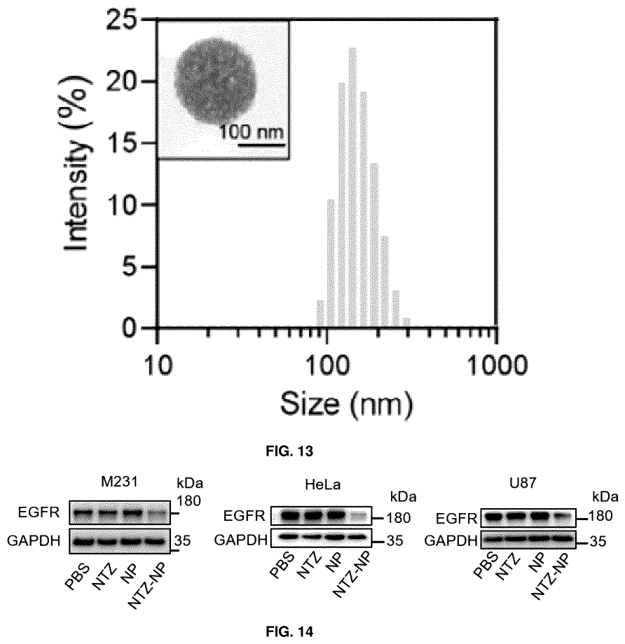

FIG. 13 of the present disclosure shows a dynamic light scattering result and a transmission electron micrograph of the third degradation tool prepared in the present example. As shown in FIG. 13, after the synthesis of the nanoparticles, the particle size of the nanoparticles is about 150 nm as measured by the dynamic light scattering, and the Polymer dispersity index (PDI) is 0.153, indicating that the obtained nanomedicine has good uniformity. The morphology of the nanoparticles is detected by a transmission electron microscope to be spherical as shown in the drawing, and the scale bar is 100 nm. The results above prove that the third degradation tool is successfully obtained.

### 3. Experiment 2: Verification of Protein Degradation Function

(1) Method of experiment 2: Nimotuzumab NTZ is human EGFR monoclonal antibody, and EGFR is tumor proliferation signal receptor and surface marker. MDA-MB-231 (M231) human breast cancer cells, HeLa human cervical cancer cells, and U87 human glioblastoma cells were treated with NTZ-NP at a final concentration of 500 nM for 24 h, followed by detection of EGFR protein expression.

(2) Result of Experiment 2:

FIG. 14 of the present disclosure shows protein electrophoresis results of protein degradation effects of the third degradation tool (NTZ-NP) in human breast cancer M231 cells, human cervical cancer HeLa cells, and human brain glioma U87 cells in Example 3. As shown in FIG. 14, after treatment of M231 cells, HeLa cells, and U87 cells with 500 nM NTZ or NTZ-NP conjugates for 24 hours, protein electrophoresis shows obvious decreases in EGFR expression. The results above prove that the nanoparticles can degrade the membrane receptor protein EGFR in various tumor cells such as breast cancer M231 cells, cervical cancer HeLa cells, and glioma U87 cells.

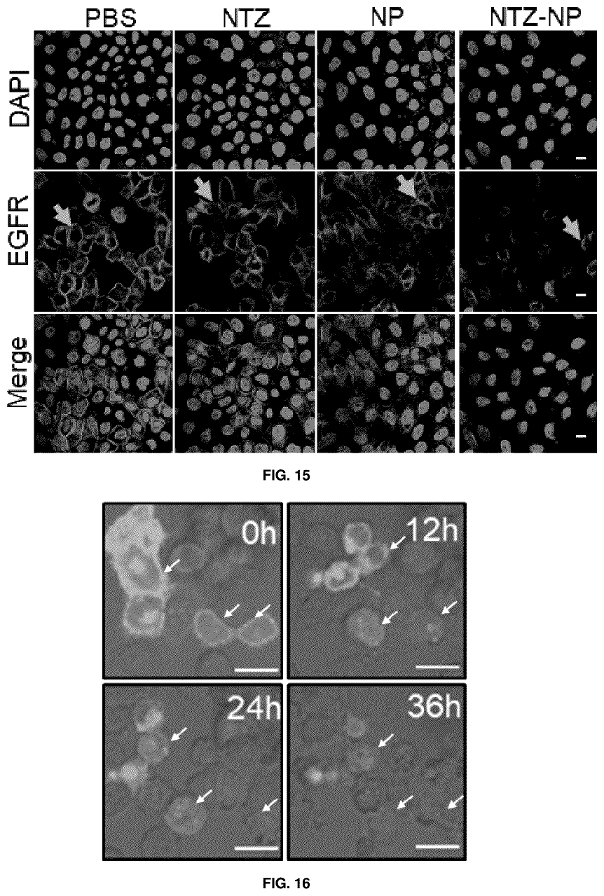

FIG. 15 of the present disclosure shows immunofluorescence staining results of M231 cells after being treated with the third degradation tool. As shown in FIG. 15, after M231 cells were treated for 24 hours with NTZ-NP at a dose of 500 nM antibodies, through immunofluorescent staining, the expression of EGFR on cell surfaces was detected by confocal microscopy, and the results show that the EGFR expression is remarkably decreased (scale bar=10 μM, arrows mark EGFR signals, DAPI is nuclear dye, and Merge is superimposed signal of EGFR and DAPI).

FIG. 16 of the present disclosure shows EGFR protein degradation results of green-fluorescence-labeled living cells chronologically photographed in a fixed visual field after M231 cells are treated by the third degradation tool. As shown in FIG. 16, in order to further clarify the EGFR protein degradation, we performed the experiment using M231 cells transfected by fusing EGFP green fluorescent protein into the intracellular domain (before terminator codon) of the EGFR. Within a fixed visual field, in a living cell culture environment, a confocal microscope photographs continuously, and the results show that the green fluorescence signal of the EGFP-labeled fused EGFR gradually decreases over time (scale bar=25 μm).

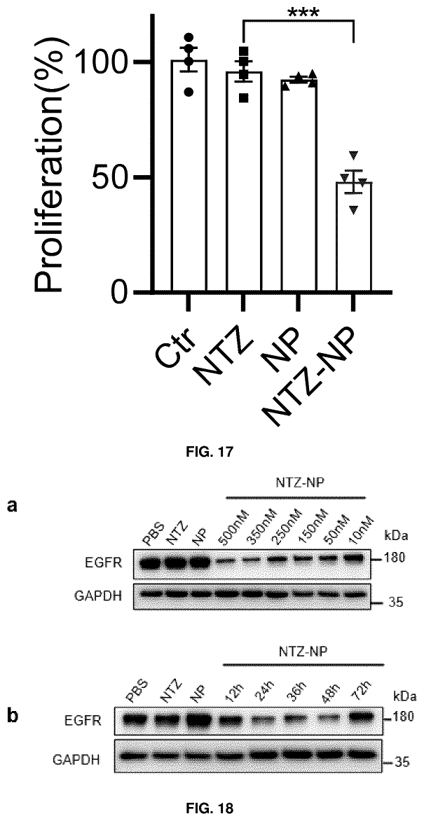

FIG. 17 of the present disclosure shows cell viability results of HepG2 cells after being treated with the third degradation tool tested by CCK8 kit. As shown in FIG. 17, after treatment of human hepatoma cells HepG2 cells with NTZ-NP at a dose of 500 nM antibody for 24 h, according to the results of the test by CCK8 cell viability assay kit, the cell proliferation thereof is significantly inhibited, ***p<0.001 has significant statistical difference.

### 4. Experiment 3: Detect Correlation Between Treatment Time and Concentration and Protein Degradation Effect

(1) Method of experiment 3: in the example, after M231 cells were treated with NTZ-NP at different concentrations for 24 hours, EGFR protein degradation thereof was detected, so as to explore an effective degradation concentration, and meanwhile, M231 cells were treated with 500 nM NTZ-NP for different periods of time.

(2) Results of experiment 3: FIG. 18 of the present disclosure shows EGFR protein expressions of the third degradation tool in Example 3, wherein FIG. 18a shows EGFR protein expression after 24 hours treatment with NTZ-NP at different concentrations, and results show that the concentration at which EGFR is effectively degraded can be as low as 10 nM, theoretically having good druggability. FIG. 18b shows protein electrophoresis of total proteins harvested after M231 cells were treated with 500 nM NTZ-NP for different periods of time, wherein from the 24th hour, the culture medium was changed to fresh culture medium without treatment so as to observe protein dynamic restoration of target protein. It can be seen that the optimal degradation time for a single treatment is 24-48 hours, and it gradually recovered after 72 hours. The performance of gradual recovery complies with the theoretical basis of the “temporary degradation” of the protein degradation tool.

### Experiment 4: Relationship Between the Ratio of NTZ-PEG (2 kDa)-DSPE Coupling Intermediate Coupled with the PLGA Core to Blank Linker without NTZ and EGFR Degradation Effect

(1) Method of experiment 4: by mixing different ratios of antibody-free PEG-DSPE linkers during the self-assembly of NTZ-PEG-DSPE and PLGA, the ratio occupied by NTZ-PEG-DSPE was adjusted, and subsequently M231 cells were treated with different NPs of 500 nM (equivalent to the molar concentration of NTZ antibody) for 24 h.

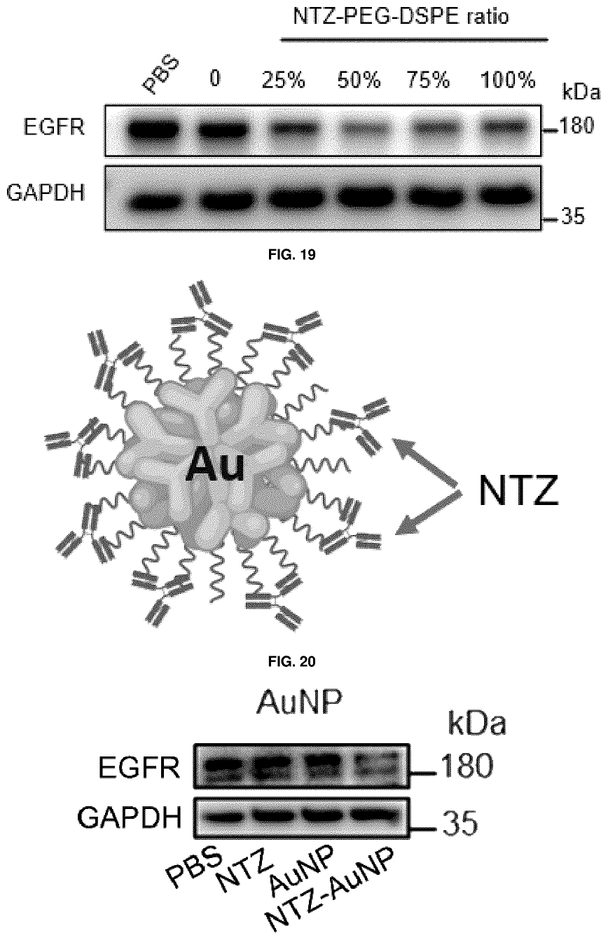

(2) Results of experiment 4: FIG. 19 of the present disclosure shows EGFR protein expressions results of the third degradation tool treatments in Example 3, which studies the influence of different ratios between the coupling intermediate composed of POI recognition groups and linkers and the blank linker in the absence of POI recognition group on the degradation effect. Results show that with the same antibody content, when the ratio of the NTZ-PEG-DSPE intermediate to the blank linker is 1:1, i.e., when the content of the NTZ-PEG-DSPE coupling intermediate is about 50%, the protein degradation effect is better. The results above show that compared with experimental group with 50% of the NTZ-PEG-DSPE arm, experimental group with 100% of the NTZ-PEG-DSPE arm may generate steric hindrance due to the NTZ of the POI recognition group, and further affect the protein degradation.

### Example 4

Referring to Table 6, in the present example, the third degradation tool was prepared, including coupling POI recognition groups with linkers, then coupling the coupled POI recognition groups and linkers with the nanoparticles to form a composite structure. The POI recognition groups were NTZ antibodies, the linkers were polyethylene glycol PEG, the nanoparticles were the composite structure, and the nanoparticles were inorganic material gold nanoparticles (AuNP), thus constituting the composite structure, i.e. “NTZ-AuNP”.

Experimental Method: Preparation of the Third Degradation Tool

Coupling of the POI recognition groups with the linkers NHS-PEG-SH (N-hydroxysuccinimide-polyethylene glycol-sulfhydryl) employed the coupling method in the first degradation tool. The POI recognition group-PEG-DSPE was coupled with the gold nanoparticles, and the coupling was performed in such a manner that a gold-sulfur bond was formed spontaneously between the sulfhydryl groups and the gold nanoparticles. Coupling methods include covalent reaction through an active group or non-covalent coupling.

Specific method: a Nimotuzumab solution was taken and concentrated by a 3 kDa ultrafiltration tube at 4000×g and replaced with 0.1 M pH=8.0 sodium bicarbonate (NaHCO₃) solution. 1 mg/mL NHS-PEG (2 kDa)-SH solution was prepared in 0.1 M, pH=8.0 NaHCO₃ solution and immediately added with nimotuzumab at a molar ratio of 1:1, the resultant was stirred on ice at 600 rpm, PEG was added dropwise, and the resultant was stirred at 4° C. and incubated overnight to obtain NTZ-PEG-SH linkers.

The coupling of AuNP and NTZ-PEG-SH (molar ratio of 1:1) was achieved by gold-sulfur bond reaction of —SH and Au. Specifically, they were stirred in PBS at 600 rpm at room temperature for 2 hours, followed by reaction overnight at 4° C. Then ultrafiltration concentration was carried out. The protein concentration was measured in IgG mode by NanoDrop one C (Thermofisher) according to the absorbance at A280, and was verified by a BCA protein assay kit.

Experimental Results:

FIG. 20 of the present disclosure shows a structural schematic diagram of the third degradation tool (NTZ-AuNP) in Example 4.

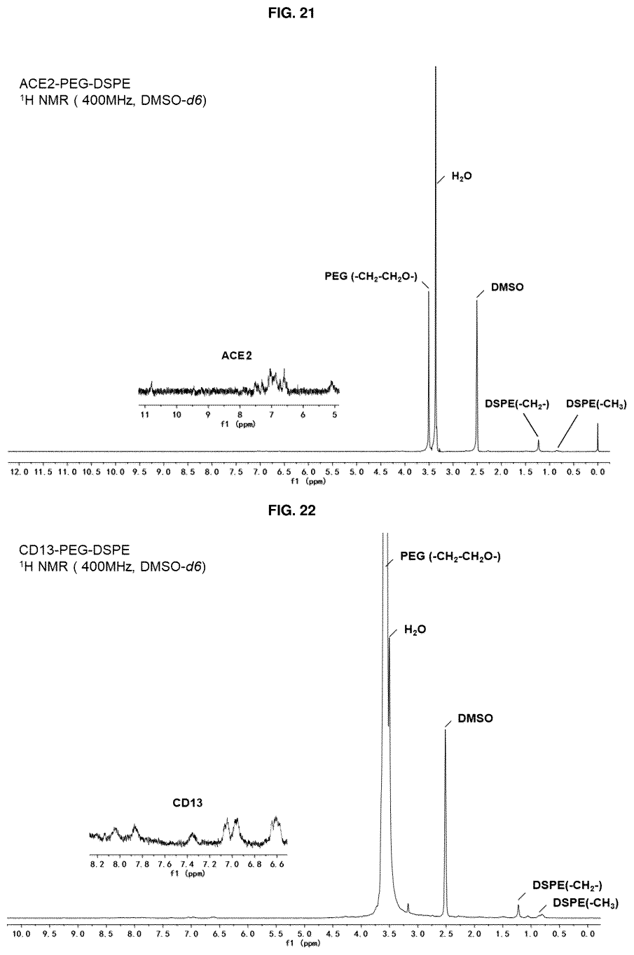

FIG. 21 of the present disclosure shows an electrophoresis result of protein degradation effect of the third degradation tool (NTZ-AuNP) in Example 4. As shown in FIG. 21, the EGFR protein expression was significantly reduced after M231 cells were treated with NTZ-AuNP and control for 24 h.

### Example 5

Referring to Table 6, in the present example, the third degradation tool was prepared, including coupling POI recognition groups with linkers, then coupling the coupled POI recognition groups and linkers with the nanoparticles to form a composite structure. In the above, the POI recognition groups were peptides having the CD13 protein or ACE2 protein or β-amyloid 1-42 oligomer binding capacity, the linkers were amphiphilic polymers (PEG-DSPE), and the nanoparticles were PLGA, thus forming the composite structure, i.e. “CD13-NP” or “ACE2-NP” or “AB-NP”.

CD13 is a coronavirus receptor and tumor marker causing common cold, and binding peptide AP-1 thereof is derived from literature and has a sequence of NH₂-YVEYHLC-COOH. ACE2 is a coronavirus receptor, and COVID19 neocoronal pneumonia virus SARS-Cov2 infects cells via ACE2 protein receptor, and binding peptide sequence NH₂-CSPLRYYPWWACT-COOH thereof is derived from literature. β-amyloid 1-42 oligomer is a potential pathogenic protein of Alzheimer's disease, which is an extracellular protein, has precursor expressed on cell membrane, and is secreted extracellularly after being sheared to form oligomers and fibers. The oligomers are considered to be the most toxic, and the accumulation of β-amyloid oligomers as extracellular protein brings about pathological risks such as neuroinflammation. The binding peptide sequence NH₂-KLVFF-COOH thereof is derived from literature.

Experimental Method: Preparation of the Third Degradation Tool

Free-NH₂/-COOH/-SH groups on the peptide or modified active residues such as azido, alkyne, maleimide or NHS were used for coupling corresponding active groups of DSPE-PEG. The coupling method of peptides is a general coupling method for coupling drugs with antibodies, including but not limited to site nonspecific coupling and specific coupling, as well as the most common amino coupling, click reaction, carboxyl coupling, thiol coupling, and selenium bond coupling.

(1) peptides and 1 equivalent (eq, molar ratio) of DSPE-PEG-reactive group powder were dissolved in 2 mL DMF solvent under nitrogen protection (the solvent does not have special requirements, and can be replaced by any other solvent that can promote peptide dissolution), and then 1.5 of triethylamine was added to the system.

(2) The resultant was stirred in a water bath at 37° C. at 800 rpm for 12 hours or more, and the reaction solution was dialyzed for 12 hours or more with 2 kDa (there are no special requirements for the molecular weight cut-off, as long as whether the coupling is successful or not can be distinguished), to remove unbound substances, then the product was lyophilized to obtain the peptide-PEG-DSPE.

(3) PBS solution of peptide-PEG-DSPE was formulated (mass ratio: PLGA:peptide-PEG-DSPE=1:4).

(4) PLGA (15 kDa) dissolved in DMF was dropwise added (2 μL per drop, with an interval of 5 seconds for each drop) into a PBS solution of peptide-PEG-DSPE stirred at 600 rpm, and the resultant was continuously stirred at room temperature for 30 minutes.

(5) The solution was centrifuged and concentrated 5 times by a 3 kDa ultrafiltration tube, wherein unbound substances were removed each time the solution was centrifuged for 4 minutes with PBS. Peptide-NP was obtained, and thereafter the concentration was determined by a BCA protein assay kit.

Experimental Results:

(1) The resulting product was dissolved in deuterated DMSO for nuclear magnetic resonance spectrometer detection.

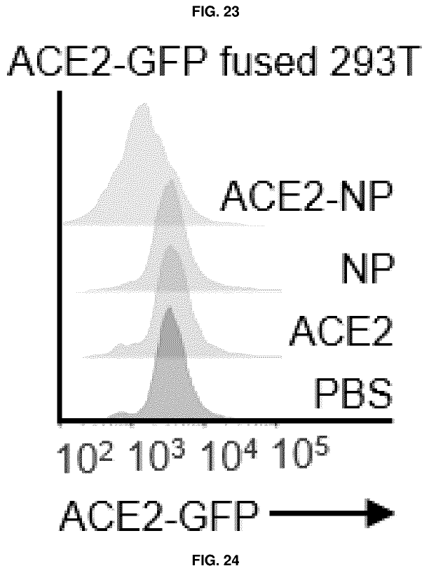

FIG. 22 of the present disclosure shows the proton nuclear magnetic resonance spectrum (¹H NMR) of coupling product (ACE2-PEG-DSPE) of POI recognition groups and linkers in the third degradation tool (ACE2-NP) in the present example. FIG. 23 of the present disclosure shows the proton nuclear magnetic resonance spectrum (¹H NMR) of coupling product (CD13-PEG-DSPE) of POI recognition groups and linkers in the third degradation tool (CD13-NP) in the present example.

As shown in FIG. 22 and FIG. 23, results of the proton nuclear magnetic resonance spectrum (¹H NMR) indicate that peptide fragments were successfully linked to PEG-DSPE in the present example.

(2)

FIG. 24 of the present disclosure shows a diagram of detection result of GFP by flow cytometry for ACE2-GFP labeled 293T cells, which are treated by the third degradation tool (ACE2-NP) in the present example. As shown in FIG. 24, after 293T cells are transfected with ACE2-GFP vector (GFP fused into intracellular domain, before terminator codon) and treated with 10 μM ACE2-binding peptide (ACE2) or NP-conjugate (ACE2-NP) thereof for 24 h, the flow cytometry results show a decrease in ACE2-GFP positive population.

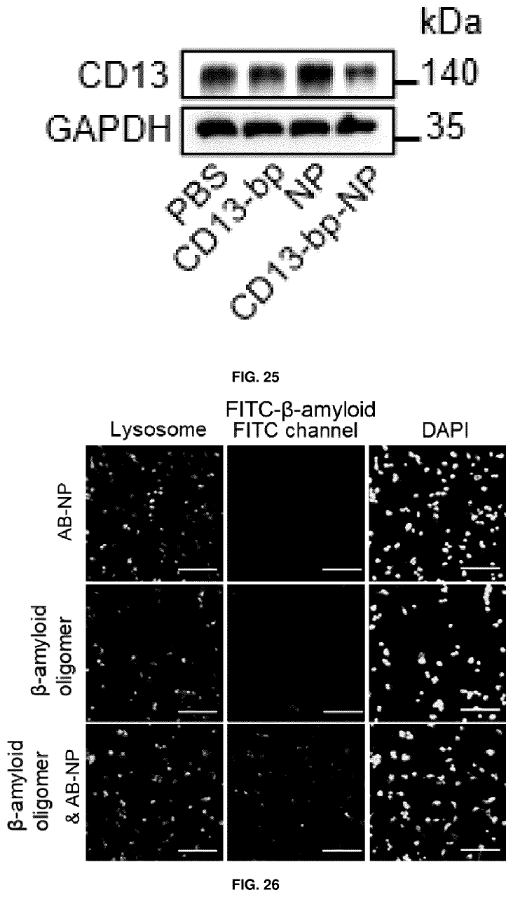

FIG. 25 shows a protein electrophoresis result of human hepatoma cell HepG2 after being treated with the third degradation tool (CD13-NP) in the present example. As shown in FIG. 25, expression of CD13 in HepG2 cells was obviously reduced after 24 h treatment with 2 μM of the CD13 binding peptide (CD13) or NP conjugate (CD13-NP) thereof.

FIG. 26 of the present disclosure shows an experimental result of endocytosis of FITC green dye-labeled β-amyloid 1-42 oligomer detected by confocal microscope after the human glial cells are treated with the third degradation tool (AB-NP) in the present example. 1 μM FITC fluorescein-labeled 5 μM β-amyloid oligomer and AB-NP were co-cultured in human glial cells for 4 hours, subsequently extracellular solution was washed away with PBS. As shown in FIG. 26, after the FITC-labeled β-amyloid oligomer was treated with AB-NP, more β-amyloids were phagocytosed, and was co-localized with the lysosome labeled with lysosome dye LysoTracker (scale bar=100 μm). The lysosome theoretically can degrade β-amyloid so as to eliminate toxicity brought about by β-amyloid extracellular accumulation.

### Example 6

Referring to Table 6, in the present example, preparation of the third degradation tool includes coupling POI recognition groups with linkers, then coupling the coupled POI recognition groups and linkers to the nanoparticles to form a composite structure. The POI recognition groups were small molecule drugs Palbociclib or small molecule probes AV-45, the linkers were amphiphilic polymers (PEG-DSPE), and the nanoparticles were PLGA.

### Experiment 1

The POI recognition groups were CDK4 inhibitor Palbociclib, and the nanoparticle conjugate thereof was Palb-NP. CDK4 is a cyclin-dependent kinase, and is widely involved in cell senescence and tumorigenesis.

Preparation Method:

(1) Palbociclib and NHS-PEG-DSPE were stirred to react under the protection of nitrogen at room temperature for 24 hours to obtain a reaction mixture of Palb-PEG-DSPE linkers, followed by dialysis and freeze-drying.

(2) Palb-PEG-DSPE-(PLGA), i.e., Palb-NP, was synthesized by transferring PLGA from organic phase to aqueous phase to achieve self-assembling, the same as the method in Example 3, PLGA was dissolved in DMF, and Palb-PEG-DSPE was dissolved in PBS.

### Experiment 2

The POI recognition groups were commercial β-amyloid probes AV-45, and the nanoparticle conjugate thereof was AV45-NP. β-amyloid is a potential pathogenic protein of Alzheimer's disease, which is an extracellular protein, has precursor expressed on cell membrane, and is secreted extracellularly after being sheared to form oligomers (β-amyloid 1-42 oligomer) and fibers, wherein the oligomers are considered to be the most toxic, and the accumulation of β-amyloid oligomers as extracellular protein brings about pathological risks such as neuroinflammation, and isotope-labeled AV-45 is a clinically commercial β-amyloid probe. In the present example, an isotope-free AV-45 derivative AV-45-SH was selected. The AV-45-SH has blue autofluorescence and is convenient for cell tracking, and the sulfhydryl SH thereof is used for coupling with Mal-PEG-DSPE.

Preparation method: binding AV45-SH with mercapto group with Mal-PEG2K-DSPE, and then binding the bound AV45-SH and mercapto group with Mal-PEG2K-DSPE with PLGA.

(1) AV45-PEG-DSPE preparation: AV45-SH and Mal-PEG2K-DSPE were dissolved in DMSO at a molar ratio of 1.2:1, and underwent water bath at 37° C. for 24 h in a light-tight condition.

(2) AV45-NP preparation: light-tight dialysis was performed for 24 h, to remove free AV45-SH, and then the resultant was linked to PLGA core. The method of experiment 1 in the present example was used as the linking method. PLGA was transferred from organic phase to aqueous phase to achieve self-assembling, AV45-PEG-DSPE was self-assembled with the PLGA in the aqueous phase. Quantitation was performed by UV absorption of AV45.

(3) β-amyloid hijack experiment: 10 μM oligomers prepared from FITC green dye-labeled 3-amyloid 1-42 peptide and 10 μM AV45-NP were co-incubated, then, the treated residue not entering the cell was washed off with PBS, followed by confocal microscope imaging, to detect green fluorescence of the β-amyloid 1-42 oligomers. Strong green fluorescence indicates that more 3-amyloids are hijacked, and theoretically, more phagocytosis and more lysosome transfers can bring about better degradation, so as to reduce toxicity caused by extracellular β-amyloid oligomer accumulation. The lysosomal dye lysoTracker was used to label the lysosome.

Experimental Results:

The concentration measurement adopts nuclear magnetism to calculate the grafting rate, and is corrected with the ultraviolet absorption standard curve.

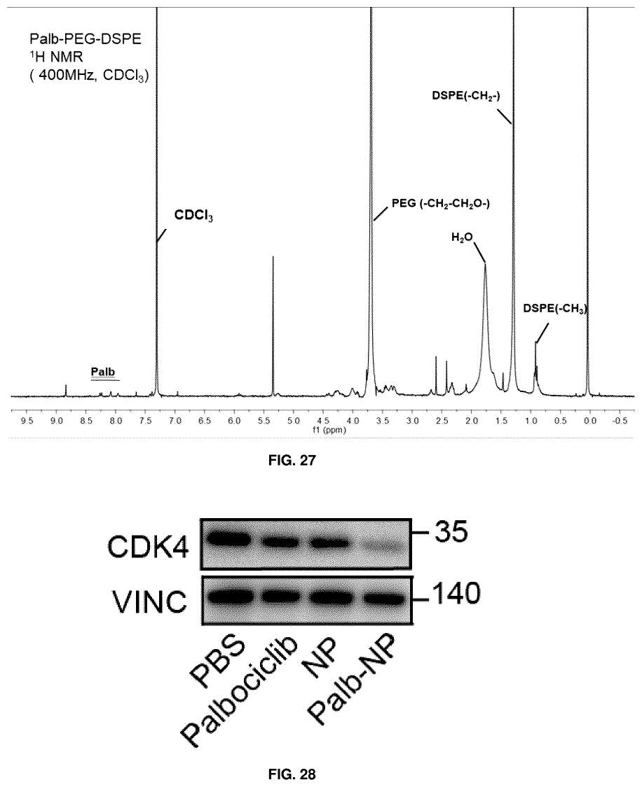

FIG. 27 of the present disclosure shows the proton nuclear magnetic resonance spectrum (¹H NMR) of coupling product (Palb-PEG-DSPE) of POI recognition groups and linkers in the third degradation tool (Palb-NP) in Example 6. The proton nuclear magnetic resonance spectrum in FIG. 27 demonstrates that the small molecule drug Palbociclib was successfully coupled with PEG-DSPE.

FIG. 28 of the present disclosure shows a protein electrophoretogram of protein degradation effect of the third degradation tool (Palb-NP) in Example 6. 2.5 μM Palbociclib or Palbociclib-coupled nanoparticles (Palb-NP) treated M231 cells for 24 hours, and proteins were harvested to detect CDK4 protein expression. Results in FIG. 28 show that Palb-NP can effectively inhibit tumor proliferation.

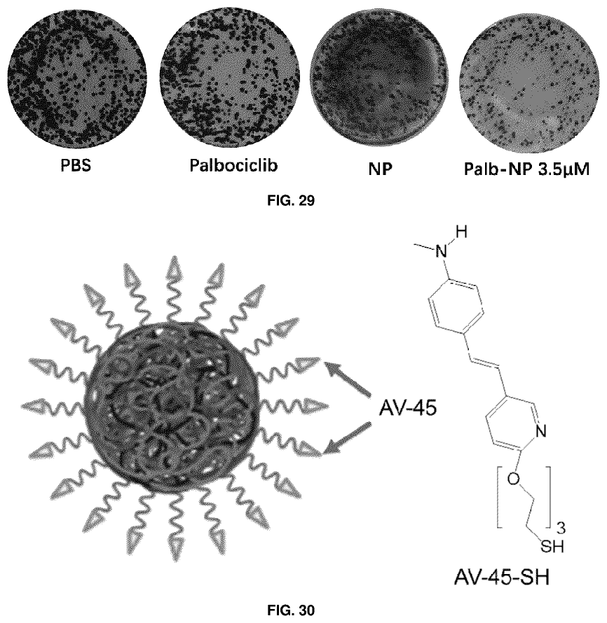

FIG. 29 of the present disclosure shows experimental results of crystal violet staining clone formation after M231 cells were treated with the third degradation tool in Example 6. After M231 cells were treated with 3.5 μM Palbociclib or Palb-NP for 48 hours, culturing was continued for 7 days, for crystal violet staining colony formation experiment. Results in FIG. 29 indicate that the effect of Palb-NP in inhibiting tumor proliferation is better than that of Palbociclib.

FIG. 30 of the present disclosure is a structural schematic diagram of AV45-NP.

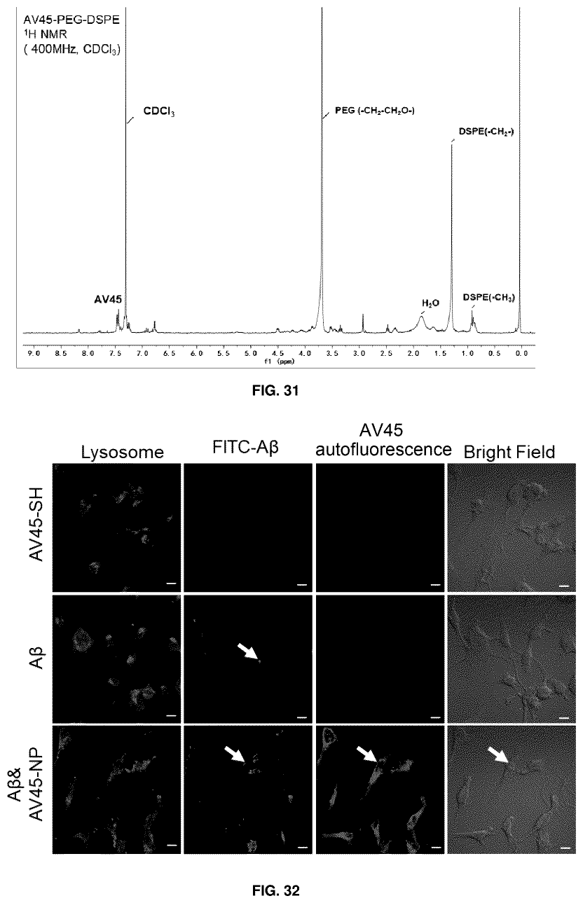

FIG. 31 of the present disclosure shows the proton nuclear magnetic resonance spectrum (¹H NMR) of coupling product (AV45-PEG-DSPE) of POI recognition groups and linkers in the third degradation tool (AV45-NP) in Example 6, and the proton nuclear magnetic resonance spectrum in FIG. 31 indicates that the small molecule drug β-amyloidare probe AV-45 is successfully linked to the PEG-DSPE.

FIG. 32 of the present disclosure shows an experimental result of endocytosis of FITC green dye-labeled β-amyloid 1-42 oligomer detected by confocal microscope after the human glial cells are treated with the third degradation tool (AV45-NP) in Example 6. 10 μM AV-45 or AV45-NP and 10 μM FITC green fluorescence dye-labeled β-amyloid oligomers were co-incubated in culture medium and treated human glial cells for 12 hours. Results of confocal microscope in FIG. 32 show that the AV45-NP group has stronger FITC green fluorescence signal and stronger AV45 autofluorescence, and is co-localized with the lyso some, indicating that the AV45-NP hijacks more 3-amyloids into human glial cells, and may promote intracellular degradation thereof. However, compared with the AV45-NP group, after treatment with AV45 alone, the autofluorescence is very weak, which also indicates that AV45-NP has a better cell internalization efficiency (scale bar in the drawing is 10 μm).

### Example 7

In the present example, the third degradation tool was prepared, obtaining CTX-NP, PTZ-NP, ATZ-NP, CRLZ-NP, and NTZ/INE-NP, respectively, including coupling POI recognition groups with linkers, then coupling the coupled POI recognition groups and linkers with the nanoparticles to form a composite structure. In the above, the POI recognition groups, the linkers, and the nanoparticles are as shown in Table 6.

Preparation method: according to the preparation method provided in Example 1, CTX-PEG-DSPE, PTZ-PEG-DSPE, ATZ-PEG-DSPE, CRLZ-PEG-DSPE, and INE-PEG-DSPE were synthesized, and according to the preparation method provided in Example 3, CTX-NP, PTZ-NP, ATZ-NP, CRLZ-NP, and INE-NP were synthesized, and NTZ/INE-NP was obtained by jointly self-assembling NTZ-PEG-DSPE and INE-PEG-DSPE in equal molar weights to PLGA, and reference is made to FIG. 8 for a structure thereof.

Experimental Results:

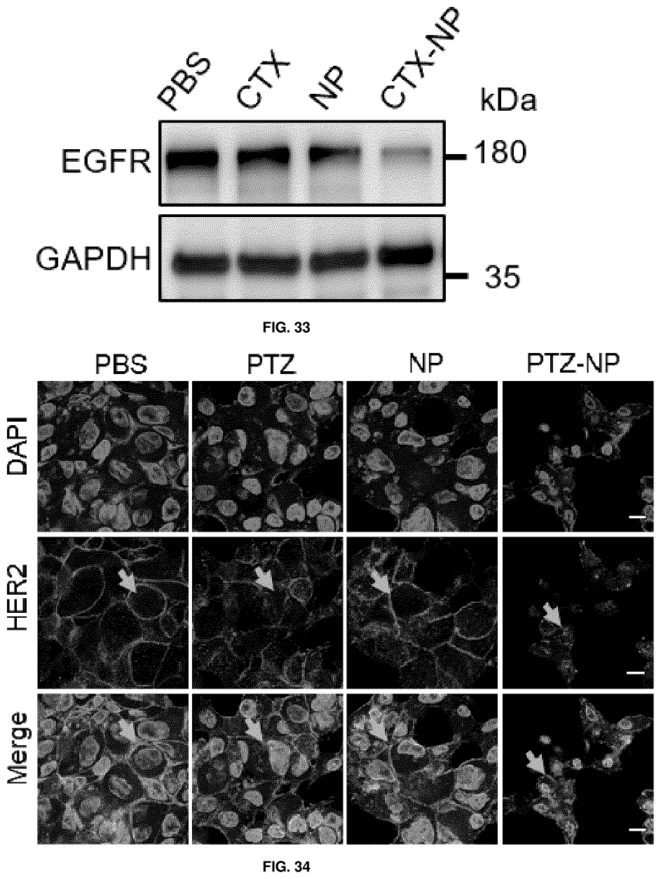

1. FIG. 33 of the present disclosure shows a protein electrophoretogram of protein degradation effect of the third degradation tool (CTX-NP) in Example 7. As shown in FIG. 33, after M231 cells were treated with 500 nM CTX-NP for 24 h, the protein electrophoresis result indicates that the expression amount of receptor protein EGFR is obviously reduced.

2. FIG. 34 of the present disclosure shows a confocal microscope image of the protein degradation effect of the third degradation tool (PTZ-NP) in Example 7. As shown in FIG. 34, after the HepG2 cells were treated with 500 nM PTZ-NP for 24 h, HER2 was subjected to immunofluorescent staining, it is found through the confocal microscope that the expression of HER2 receptor protein in the PTZ-NP group is obviously reduced (the scale bar=10 μm, and membrane structures marked by arrows are HER2).

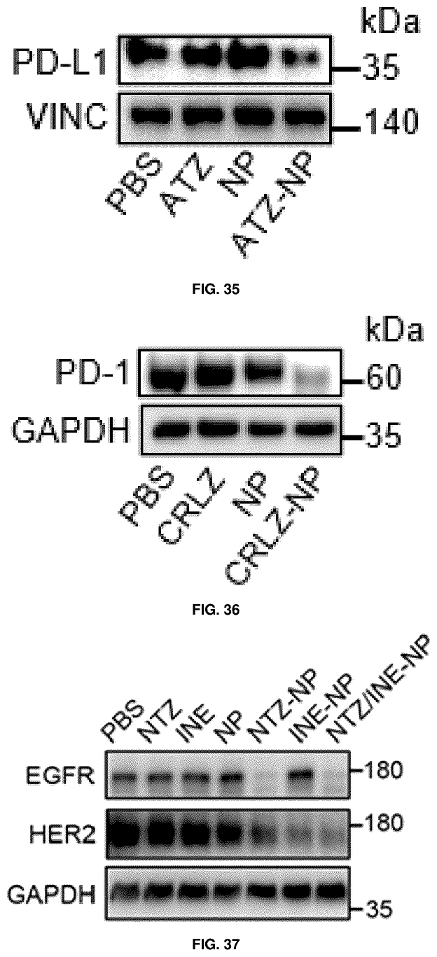

3. FIG. 35 of the present disclosure shows a protein electrophoretogram of protein degradation effect of the third degradation tool (ATZ-NP) in Example 7. As shown in FIG. 35, after M231 cells were treated with 500 nM CTX-NP for 24 h, the protein electrophoresis result shows that the expression amount of receptor protein EGFR was obviously reduced.

4. FIG. 36 of the present disclosure shows a protein electrophoretogram of protein degradation effect of the third degradation tool (CRLZ-NP) in Example 7. As shown in FIG. 36, after JURKAT cells overexpressing PD-1 were treated with 500 nM CRLZ-NP for 24 h, the protein electrophoresis result shows that the expression of PD-1 receptor protein was obviously reduced.

FIG. 37 of the present disclosure shows a protein electrophoretogram of protein degradation effect of the third degradation tool (INE/NTZ-NP) in Example 7. As shown in FIG. 37, after HER2-expressed 293T cells were treated with 500 nM INE-NP or NTZ/INE-NP for 24 h, the protein electrophoresis results show that the expression of HER2 and EGFR receptor proteins was obviously reduced. This indicates that the present disclosure can obtain the capability of multi-specific targeting in a simple way of “plug and play”, and has a relatively good application potential.

### Example 8

### 1. Experiment 1: Animal In Vivo Therapeutic Effect of NTZ-NP on Subcutaneous Tumor Implantation Model of MDA-MB-231 Breast Cancer

The present example employed the preferred third degradation tool obtained in Example 3, wherein the POI recognition groups were NTZ, the linkers were DSPE-PEG, and the nanoparticle core was PLGA. In the present example, the animal in vivo therapeutic effect of NTZ-NP on subcutaneous tumor implantation model of MDA-MB-231 (M231) breast cancer was explored. Administration every other day (15 mg/kg) was started on day 10 of inoculation of M231 tumor in female nude mice at 6-8 weeks of age, and a volume of the tumor (tumor length: a and width: b, volume calculation formula: ½ab²) of the mice and body weight change were continuously monitored. After two weeks of administration, 7 times in total, the mice were euthanized the next day after the last administration, tumor tissues were isolated, and the tumor tissues were subjected to protein electrophoresis and immunofluorescence detection.

Experimental Results:

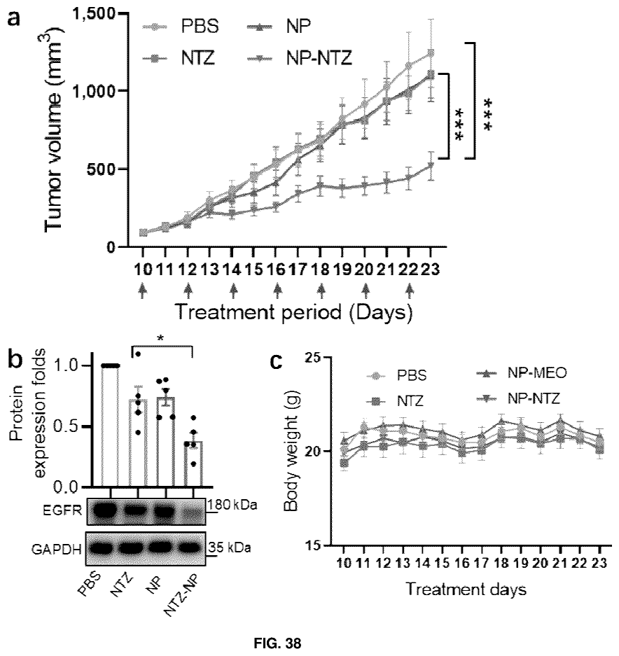

In FIG. 38 of the present disclosure, FIG. 38a shows the tumor volumes of nude mice during NTZ-NP treatment on M231 cells in nude mouse subcutaneous tumor models. The experimental results as shown in a of FIG. 38 show that the tumor volume proliferation was obviously inhibited in the NTZ-NP group compared with the solvent PBS control group, the equal molar NTZ antibody group, and the equal molar antibody-free nanoparticle group.

FIG. 38b shows the EGFR protein expression level detected by protein electrophoresis after the tumor tissues are isolated, and it can be seen that EGFR is significantly down-regulated. The treated tumor tissues of each kind were taken from five different experimental mice and subjected to protein electrophoresis and grey-scale statistics relative to its own internal reference, and a statistical result *p<0.05 has statistical difference.

FIG. 38c shows the changes of body weight of the mice during the NTZ-NP treatment, and results show no obvious change in body weight.

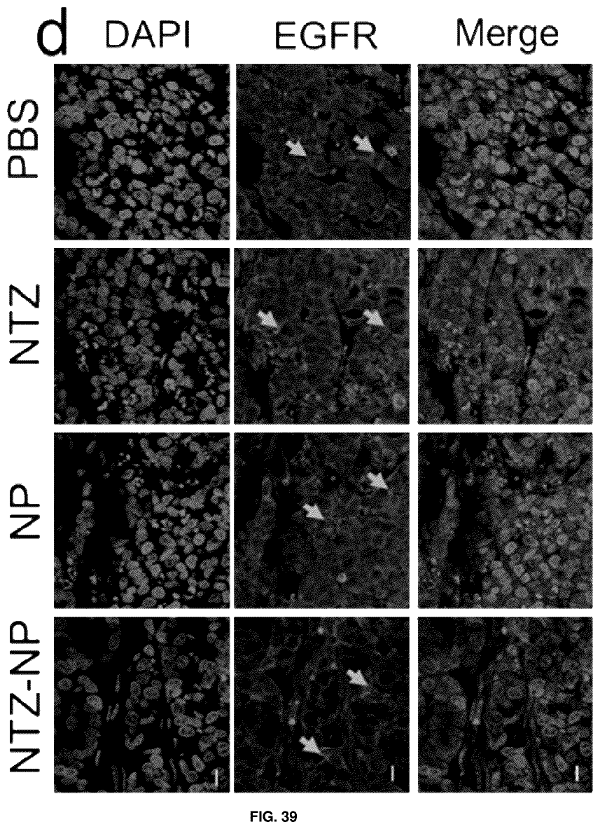

FIG. 39 shows results of EGFR detected by immunofluorescent staining for isolated tumor tissues after the NTZ-NP treatment is completed.

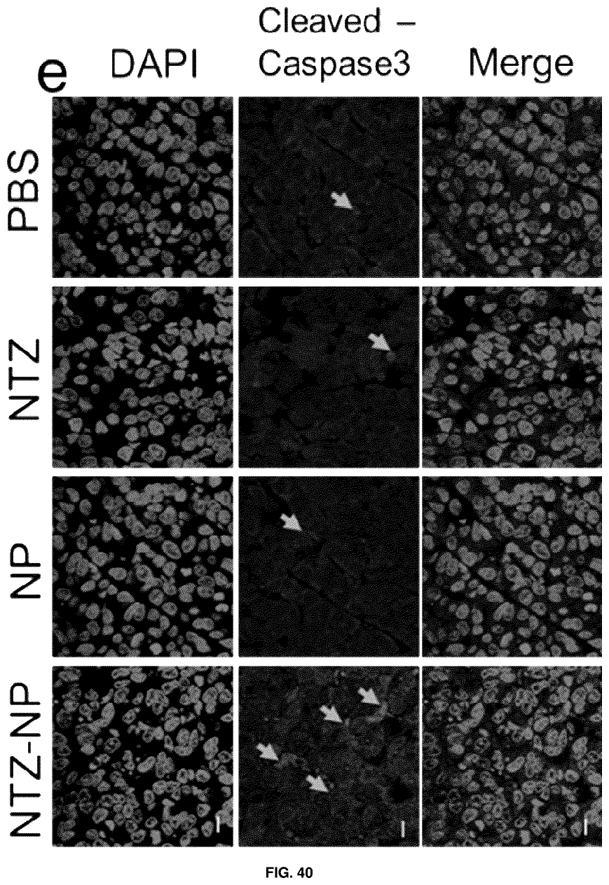

FIG. 40 shows results of apoptotic markers detected by immunofluorescent staining for isolated tumor tissues after the NTZ-NP treatment is completed. In the above, after being isolated, the tumor tissues were paraffin-embedded and sectioned, followed by immunofluorescence staining, and immunofluorescence staining results of EGFR and apoptotic core signal cleaved caspase-3 were detected. It can be seen that in the NTZ-NP group the EGFR signal is reduced, and apoptotic signal rises. Results show that the NTZ-NP group nanoparticles can degrade the growth factor receptor protein EGFR on the surface of mouse tumor cells, and the NTZ-NP has therapeutic effect on the mouse subcutaneous tumors, and shows in vivo application potential.

### 2. Experiment 2: Acute Hepatotoxicity Research on Mice by NTZ-NP Nano-Structural Protein Degradation Tool

The present example employed the preferred third degradation tool obtained in Example 3, wherein the POI recognition groups were NTZ, the linkers were DSPE-PEG, and the nanoparticle core was PLGA. In the present example, acute hepatotoxicity on mice by the NTZ-NP nano-structural protein degradation tool was studied. The nanoparticles, the solvent PBS control, NTZ antibodies of equal molar quantity, and NP of equal molar quantity without NTZ antibodies (10 mg/kg) were injected into mice via tail vein, blood was taken 12 hours after injection, and blood biochemical indicators were detected by a kit.

Experimental Results:

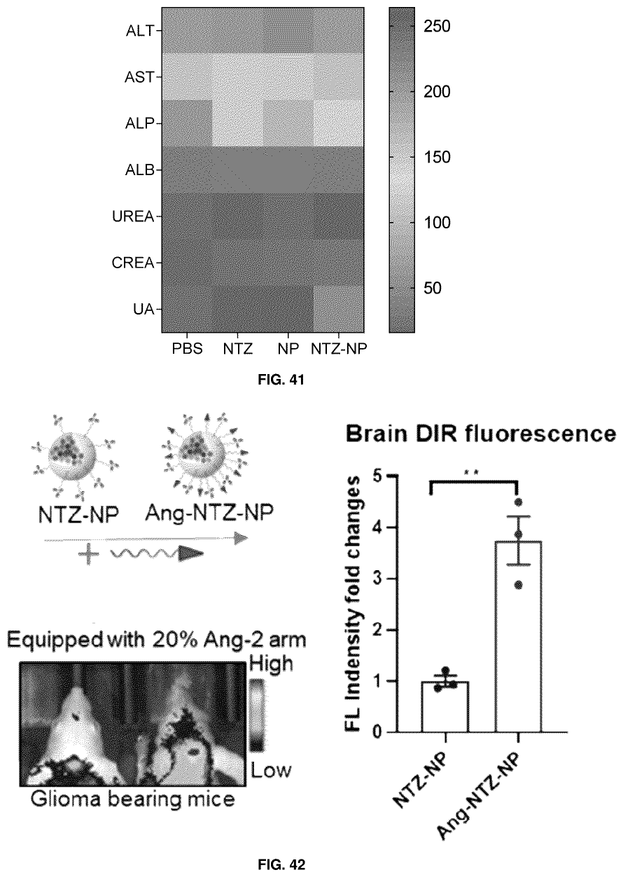

FIG. 41 of the present disclosure shows detection of blood parameters of liver function and kidney function of mice after a single administration of the third degradation tool (NTZ-NP) in Example 8. As shown in FIG. 41, the experimental results show that relevant indexes such as blood urea nitrogen (BUN), uric acid (UA), creatinine (CR), albumin (ALB), alkaline phosphatase (ALP), alanine aminotransferase (ALT), and aspartate aminotransferase (AST) have no obvious abnormality, indicating that the nanoparticles have no obvious toxic and side effect on liver and kidney metabolism of mice.

### 3. Experiment 3: Whether NTZ-NP can Obtain Blood-Brain Barrier Crossing Capacity and Tumor Targeting Capacity by Simple “Plug and Play”

The present example employed the preferred third degradation tool obtained in Example 3, wherein the POI recognition groups were NTZ, the linkers were DSPE-PEG, and the nanoparticle core was PLGA.

In the present example, it is explored whether NTZ-NP can obtain the blood-brain barrier crossing capacity and tumor targeting capacity by simple “plug and play”.

DIR-carried NTZ-NP was obtained by wrapping near infrared dye DIR that cannot pass through the blood-brain barrier and cannot target tumor cells in the PLGA core (after mutual solubility of organic phases, DIR and PLGA were added dropwise to an NTZ-PEG-DSPE aqueous solution to self-assemble, followed by purification and concentration).

When self-assembling the NTZ-PEG-DSPE arms to the PLGA core, 20% of Angiopep2-PEG-DSPE with the blood-brain barrier crossing capacity was added, to be self-assembled together with DIR-carried PLGA, followed by purification and concentration, to obtain DIR-carried Ang-NTZ-NP (wherein the angiopep-2 peptide is a representative blood-brain barrier crossing peptide and tumor targeting peptide, and of which a peptide sequence (NH₂-TFFYGGSRGKRNFKTEEY-COOH) is from literature.

Two kinds of nanoparticles (antibody amount of 10 mg/kg, and DIR amount of 0.25 mg/kg) of equal molar quantity were intravenously injected into tumor-bearing nude mice (human brain glioma in situ transplantation), and through a small animal imager IVIS system, DIR fluorescence is compared to show the blood-brain barrier crossing capacity and the tumor targeted accumulation effect.

Experimental Results:

FIG. 42 shows the capacity of crossing blood-brain barrier and tumor targeting activity of the third degradation tool (NTZ-NP) obtained by simple self-assembling in Example 8 of the present disclosure, which can be traced due to the loading of fluorescent dye DIR, reflected by the brain accumulation of DIR fluorescence detected by a small animal imager and statistical chart.

The left drawings in FIG. 42 show that when the glioma tumor-bearing mice were injected with the nano degradation tool Ang-NTZ-NP containing 20% Ang-2 arms, DIR dye signals in the brain were obviously aggregated, the right drawing shows statistics of three repeated biological experiments, and the right drawing shows that the level of in-brain Ang-NTZ-NP accumulation was obviously improved, which has significant difference.

Results prove that the TPD-NP nanoparticles have drug-carrying capacity, and can also be modified and self-assembled simply by plug and play, so as to obtain-blood brain barrier crossing capacity and tumor targeting capacity.

### 4. Experiment 4: It is Studied Whether NTZ-NP can Obtain the Blood-Brain Barrier Crossing Capacity and Tumor Targeting Capacity by Simple “Plug and Play”, and Perform Protein Degradation of Target Protein EGFR and Tumor Inhibiting on In Situ Tumor Models, and the In Vivo Therapeutic Potential is Studied

The present example employed the preferred third degradation tool obtained in Example 3, wherein the POI recognition groups were NTZ, the linkers were DSPE-PEG, and the nanoparticle core was PLGA.

In the present example, it is explored whether NTZ-NP can obtain the blood-brain barrier crossing capacity and tumor targeting capacity by simple “plug and play”, and perform protein degradation and tumor inhibiting and killing of target protein EGFR on in situ tumor models, and the in vivo therapeutic potential is studied.

The preparation method is as described in experiment 3 of the present example, wherein when self-assembling the NTZ-PEG-DSPE arms to the PLGA core, 20% of Angiopep2-PEG-DSPE with the blood-brain barrier crossing capacity was added, to be self-assembled together with PLGA, followed by purification and concentration, to obtain Ang/NTZ-NP 15 days after human glioma was transplanted to 6-8 weeks old female nude mice, 10 mg/kg intravenous administration was carried out, and control of equal molar quantity and solvent control of equal volume were also injected intravenously at the same time. The administration was performed once every other day, 5 times in total, and after the administration cycle was completed, the mice were euthanized the next day, the brain tissues were taken and paraffin-embedded, and sectioned for immunohistochemical staining to study the protein degradation effect of NTZ on target protein EGFR and the tumor inhibition by detecting the tumor proliferation marker PCNA.

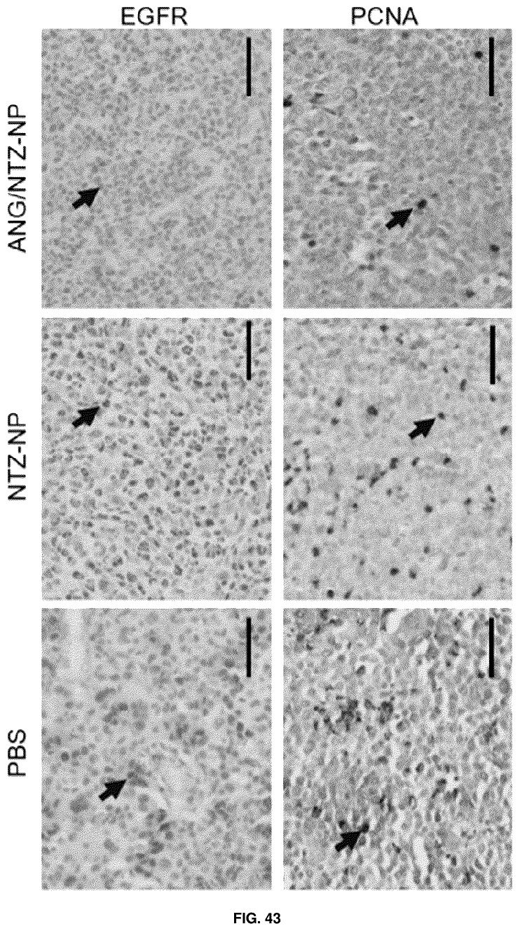

FIG. 43 shows diagrams of results of immunohistochemical detection of EGFR and proliferation marker PCNA after brain tumor dissection after the third degradation tool (NTZ-NP) of Example 8 is administered to glioma in situ animal models by the assembling method in FIG. 42. The results show that EGFR and PCNA in the solvent PBS control group and the NTZ-NP group are stronger than the ANG/NTZ-NP group. It indicates that the nano-structural protein degradation tool has a better application potential (arrows mark representative positive signals. The scale bar is 50 μm).

### Example 9

With reference to Table 7, in the present example, the degradation tool in Example 9 was prepared, including coupling the POI recognition groups with the lipid hybrid substances, wherein the POI recognition groups were monoclonal antibody drug nimotuzumab (NTZ); the lipid hybrid substances were PEG (polyethylene glycol) liposomes, i.e., the liposomes contained PEG exposed on the surface of the lipid hybrid substances, with PEG terminal thereof having an active reaction site NHS (N-hydroxysuccinimide) for coupling amino of the POI recognition group NTZ antibody. A product obtained in the present example has a structure of NTZ-PEGlipo.

Preparation Method:

(1) pretreatment: replacing a buffer solution of original antibodies with PBS. In the present example, EGFR monoclonal antibody Nimotuzumab (NTZ) which has been applied to clinical treatment was used.

A nimotuzumab solution was centrifuged by a 3 kDa ultrafiltration tube at 4000×g for 2 minutes, then the antibodies were concentrated, and then diluted with PBS; after repeated concentration and dilution, the buffer solution was replaced with PBS, after dilution, protein concentration was determined in IgG mode by NanoDrop one C instrument according to the absorbance at A280, and the result thereof was verified by a BCA protein quantification kit.

(2) Preparation of the lipid hybrid substances: the lipid hybrid substances used were liposomes, composed of hydrogenated soy phosphatidylcholine (HSPC), cholesterol (CHO), DSPE (distearoyl phosphatidyl ethanolamine)-PEG (polyethylene glycol) (the molecular weight of polyethylene glycol is 2 kDa) by the classical membrane hydration method, wherein a mass ratio of HSPC:CHO:DSPE-PEG=56:39:2.5. HSPC and CHO were dissolved in chloroform, and subsequently evaporated, concentrated, and dried to form a lipid membrane. After complete evaporation of chloroform, 1 mL of PBS solution of DSPE-PEG-NHS was added, followed by ultrasonic treatment at 100 W for 3 min at room temperature. The resulting solution was passed through 800 nm, 400 nm, and 200 nm filter membranes, respectively, to insert DSPE-PEG-NHS into the lipid membrane, which process should be completed within 10 min.

(3) Coupling of the POI recognition group NTZ antibodies with the PEG lipid hybrid substances:

The coupling reaction was started in an ice-water mixture environment, and was protected with nitrogen. The lipid hybrid substance solution was then added to the antibody PBS solution stirred at 800 rpm (revolutions per minute); and the resultant was then stirred and reacted for incubation on a 20 rpm rotator at 4° C. for 24 hours.

(4) Purification: reaction mixture was then subjected to ultrafiltration concentration by a 50 kDa centrifuging ultrafiltration tube for 3 times, followed by dosing to 100 μL with PBS. Protein concentration was determined in IgG mode by NanoDrop one C instrument according to the absorbance at A280, and the result thereof was verified by a BCA protein quantification kit.

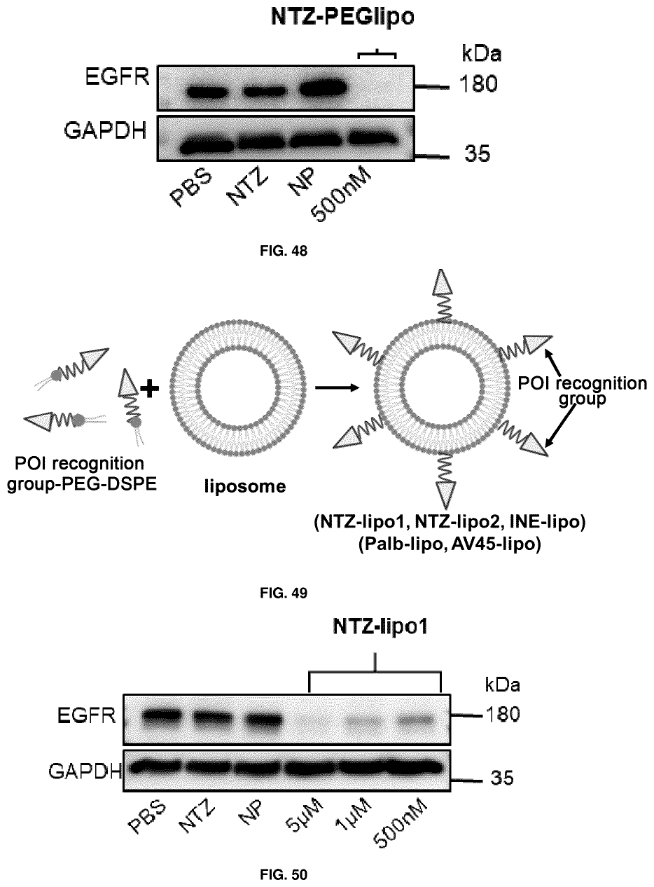

Experimental results: FIG. 47 is a diagram of an NTZ-PEGlipo synthesis process. FIG. 48 shows results of EGFR protein expression detected by protein electrophoresis of the harvested proteins after MDA-MB-231 (M231) human breast cancer tumor cells are treated with 500 nM NTZ-PEGlipo for 24 hours, and the result shows that the NTZ-PEGlipo can effectively degrade the target protein EGFR.

### Example 10

Referring to Table 7, in the present example, a degradation tool in Example 10 was prepared, i.e. coupling the POI recognition groups with the amphiphilic lipid linkers, and then self-assembling the coupled POI recognition groups and the amphiphilic lipid linkers with lipid hybrid substances. In Example 10a and Example 10b, the POI recognition group was monoclonal antibody drug nimotuzumab (NTZ); and the linker was NHS-PEG-DSPE with NHS group. The lipid hybrid substance was composed of a membrane skeleton and cholesterol (CHO). The membrane skeleton in Example 10a was hydrogenated soy phosphatidylcholine (HSPC), and the degradation tool formed was NTZ-lipo1. The membrane skeleton in Example 10b was distearoyl phosphatidylcholine (DSPC), and the degradation tool formed was NTZ-lipo2. In Example 10c, the POI recognition group was therapeutic monoclonal antibody Inetetamab (INE) of HER2, wherein HER2 is a tumor proliferation signal receptor and marker, the linker was NHS-PEG-DSPE with NHS group, the lipid hybrid was composed of an HSPC membrane skeleton and CHO, and the degradation tool formed was INE-lipo.

In Example 10d, the POI recognition group was Palbociclib (Palb), which was a small-molecule inhibitor for CDK4 and CDK6, the linker was NHS-PEG-DSPE with NHS group, Palbociclib was coupled by reaction of amino group with NHS, the lipid hybrid substance was composed of an HSPC membrane skeleton and CHO, and the degradation tool formed was Palb-lipo.

Preparation Method:

Experiment 1: the POI recognition group was an antibody. In Example 10a and Example the POI recognition group was monoclonal antibody drug nimotuzumab (NTZ). In Example the POI recognition group was monoclonal antibody drug nimotuzumab (INE).

(1) pretreatment: replacing a buffer solution of original antibodies with a phosphate buffer solution PBS. In the present example, an NTZ and INE monoclonal antibody solution was centrifuged by a 3 kDa ultrafiltration tube at 4000×g for 2 minutes, then the antibodies were concentrated, and subsequently diluted with PBS; after concentration and dilution were repeated three times, main components of the buffer solution were replaced with PBS, after the antibody solution was diluted, protein concentration was determined in IgG mode by NanoDrop one C instrument according to the absorbance at A280, and the result thereof was verified by a BCA protein quantification kit.

(2) Coupling reaction between POI recognition group and amphipathic lipid linkers: the antibodies were reacted with NHS-PEG (2 kDa)-DSPE through amino groups on the antibodies and NHS (N-hydroxysuccinimide) groups at DSPE-PEG terminals. The coupling reaction was started in an ice-water mixture environment, and was protected with nitrogen. In order to improve the dispersity, it can be selected to perform ultrasonic treatment on the DSPE-PEG in the PBS buffer solution at 40 kHz for 3 minutes, and then NHS-PEG-DSPE PBS solution of corresponding molar concentration and volume (the molar ratio is not limited to 1:1, dependent on the antibody-drug conjugate) was added to the antibody PBS solution stirred at 800 rpm (revolutions per minute); then the resultant mixture was stirred and reacted for incubation on a 20 rpm rotator at 4° C. for 24 hours (in order to maintain antibody activity, it tends to be carried out at low speeds and 4° C.). An NTZ-PEG-DSPE reaction mixture or an INE-PEG-DSPE reaction mixture was obtained.

In the above, the coupling method includes, but is not limited to, non-site-fixed coupling and site-fixed coupling. The non-site-fixed coupling includes amino coupling, carboxyl coupling, bridging thiol coupling. The site-fixed coupling includes, for example, click reaction, selenium bond coupling, serine coupling, cysteine coupling, unnatural amino acid coupling, enzyme-catalyzed coupling, and sugar site coupling.

(3) Purification: The reaction mixture containing NTZ-PEG-DSPE or INE-PEG-DSPE was then subjected to ultrafiltration concentration by a 50 kDa centrifuging ultrafiltration tube for 3 times, followed by dosing to 100 μL with PBS. Protein concentration was determined in IgG mode by NanoDrop one C instrument according to the absorbance at A280, and the result thereof was verified by a BCA protein quantification kit.

(4) Preparation of the lipid hybrid substances: the lipid hybrid substances used were liposomes, composed of hydrogenated soy phosphatidylcholine (HSPC) or distearoyl phosphatidylcholine (DSPC), cholesterol (CHO), and DSPE-PEG (2 kDa) by the classical membrane hydration method, wherein a mass ratio of [HSPC or DSPC]:CHO:DSPE-PEG (not containing POI recognition group)=56:39:2.5. HSPC or DSPC and CHO were dissolved in chloroform, and then evaporated, concentrated, and dried to form a membrane.

Preparation of NTZ-lipo1, NTZ-lipo2, and INE-lipo: after complete evaporation of solvent chloroform of the preceding lipid hybrid substance solution, 1 mL of PBS solution of DSPE-PEG-antibody was added, followed by ultrasonic treatment at 100 W for 3 min at room temperature. The resulting solution was then passed through 800 nm, 400 nm, and 200 nm filter membranes, respectively. Subsequently further purification was carried out, reaction mixture was subjected to ultrafiltration concentration by a 50 kDa centrifuging ultrafiltration tube for 3 times, followed by dosing to 100 μL with PBS. Protein concentration was determined in IgG mode by NanoDrop one C instrument according to the absorbance at A280, and the result thereof was verified by a BCA protein quantification kit.

Subsequently, cell experiment was carried out according to the antibody concentration.

### Experiment 2: The POI Recognition Group was Small Molecule. In Example 10d, the POI Recognition Group was Palbociclib Small Molecule Drug or β-Amyloid Probe AV-45

Palbociclib is a clinically commercial CDK 4 inhibitor. In the example, a conjugate of the lipid hybrid substance was Palb-lipo. CDK4 is a cyclin-dependent kinase, and is widely involved in cell senescence tumorigenesis.

Palbociclib and NHS-PEG (2 kDa)-DSPE reacted with the NHS group at the terminal of DSPE-PEG through the amino group on Palbociclib (molar ratio 1.2:1). Under the protection of nitrogen at room temperature, stirring and reaction were carried out for 24 hours to obtain a reaction mixture Palb-PEG-DSPE. The Palb-PEG-DSPE was dialyzed for 24 hours and freeze-dried, and the concentration was determined according to the standard curve of ultraviolet absorption peak.

Preparation of the lipid hybrid substances was the same as in experiment 1 of the present example.

Preparation of Palb-lipo: after complete evaporation of the solvent chloroform in the preceding lipid hybrid substances, 1 mL of PBS solution of Palb-PEG-DSPE was added, followed by ultrasonic treatment at 100 W for 3 min at room temperature. The resulting solution was then passed through 800 nm, 400 nm, and 200 nm filter membranes, respectively. The concentration was determined according to the standard curve of ultraviolet absorption peak, and the cell experiment was carried out according to Palb molar concentration.

AV-45 is a probe for β-amyloid, which is a classic marker of Alzheimer's disease, and a lipid hybrid substance conjugate thereof is Av45-lipo.

3-amyloid is a potential pathogenic protein of Alzheimer's disease, which is an extracellular protein, has precursor expressed on cell membrane, and is secreted extracellularly after being sheared to form oligomers (β-amyloid 1-42 oligomer) and fibers, wherein the oligomers are considered to be the most toxic, and the accumulation of β-amyloid oligomers as extracellular protein brings about pathological risks such as neuroinflammation, and isotope-labeled AV-45 is a clinically commercial β-amyloid probe. In the present example, an isotope-free AV-45 derivative AV-45-SH was selected. The AV-45-SH has blue autofluorescence and is convenient for cell tracking, and the sulfhydryl SH thereof is used for coupling with Mal(maleimide)-PEG-DSPE.

Preparation method of AV45-PEG-DSPE: binding AV45-SH with mercapto group with Mal-PEG (molecular weight 2 kDa)-DSPE, i.e., dissolving AV45-SH and Mal-PEG-DSPE in DMSO at a molar ratio of 1.2:1, and being subjected to light-tight water bath with nitrogen protection at 37° C., subsequently followed by dialysis and freeze-drying, and determining the concentration according to the standard curve of ultraviolet absorption peak.

The preparation of the lipid hybrid substances was the same as in experiment 1 of the present example. Preparation of AV45-lipo: after complete evaporation of solvent chloroform of the preceding lipid hybrid substance solution, 1 mL of PBS solution of AV45-PEG-DSPE was added, followed by ultrasonic treatment at 100 W for 3 min at room temperature. The resulting solution was then passed through 800 nm, 400 nm, and 200 nm filter membranes, respectively. The concentration was determined according to the standard curve of ultraviolet absorption peak, and the cell experiment was carried out according to AV45 molar concentration.

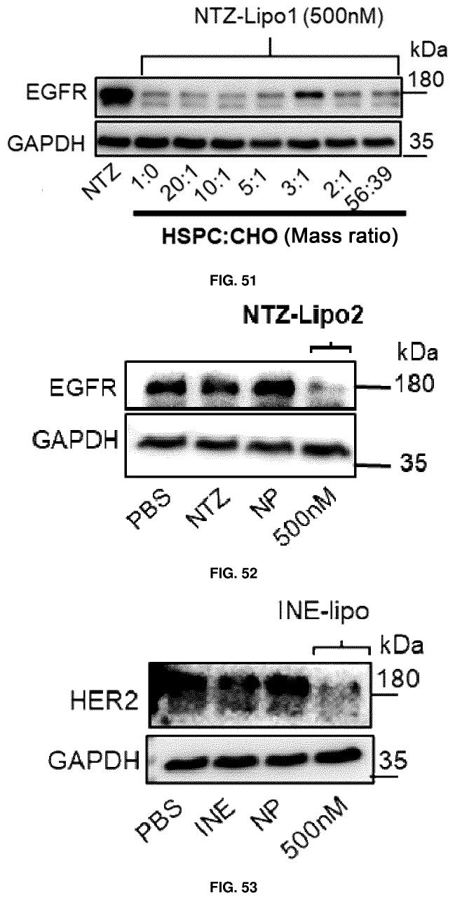

Experimental results: FIG. 49 is a schematic diagram of a synthesis process. FIG. 50 shows results of EGFR protein expression detected by protein electrophoresis of the harvested proteins after M231 human breast cancer tumor cells were treated with NTZ-lipo1 at different concentrations for 24 hours, and the results show that the NTZ-lipo1 can effectively degrade the target protein EGFR. FIG. 51 shows protein electrophoresis results of effect on target protein EGFR degradation by treatment of M231 cells for 24 hours with NTZ-lipo1 of lipid hybrid substances composed of HSPC and CHO at different ratios, and the results show that different compositions have the protein degradation effect, while the ratio of membrane skeleton HSPC to auxiliary lipid cholesterol has influence on the protein degradation effect. FIG. 52 shows results of EGFR protein expression detected by protein electrophoresis of the harvested proteins after M231 human breast cancer tumor cells were treated with 500 nM NTZ-lipo2 for 24 hours, and the results show that the NTZ-lipo2 can effectively degrade the target protein EGFR. FIG. 53 shows results of HER2 protein expression detected by protein electrophoresis of the harvested proteins after M231 human breast cancer tumor cells were treated with 500 nM INE-lipo for 24 hours, and the results show that the INE-lipo can effectively degrade the target protein.

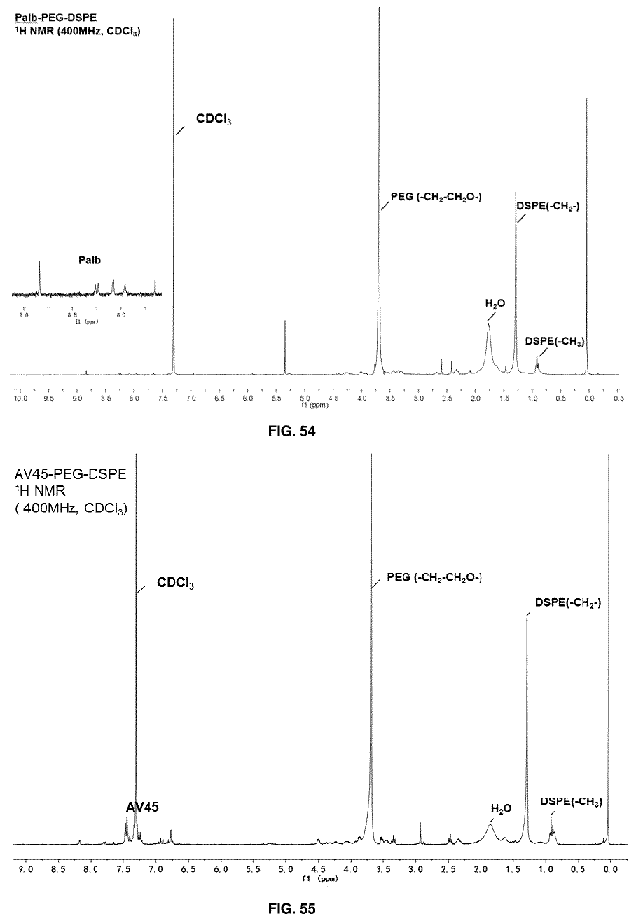

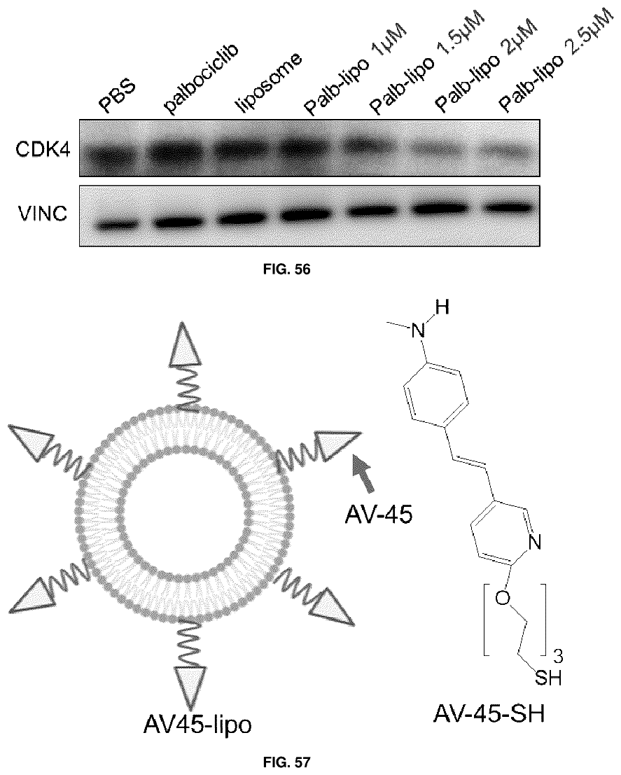

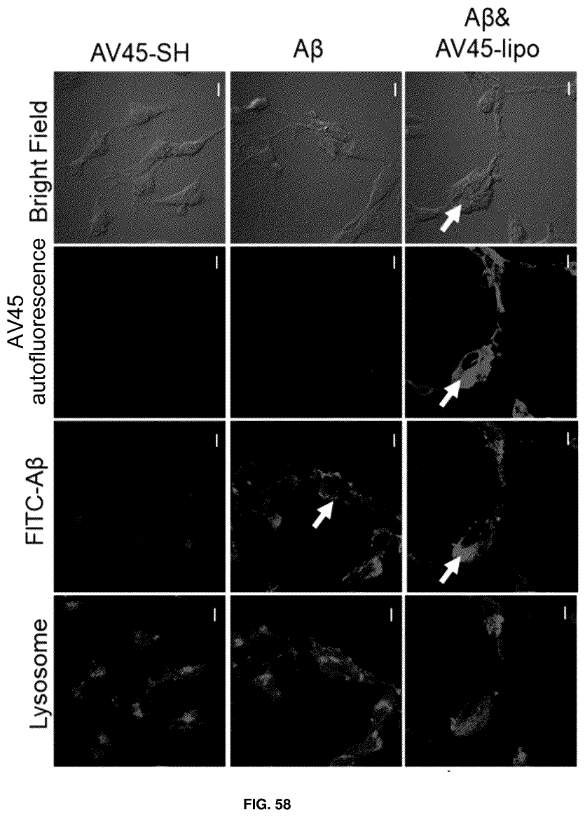

FIG. 54 shows the proton nuclear magnetic resonance spectrum (¹H NMR) of Palb-PEG-DSPE after coupling of the lipid linkers and the POI recognition groups used by Palb-lipo, and results show successful linkage. FIG. 55 shows the proton nuclear magnetic resonance spectrum (¹H NMR) of AV45-PEG-DSPE after coupling of the lipid linkers with the POI recognition groups used by AV45-lipo, and results show successful linkage. FIG. 56 shows CDK4 protein expression results detected by protein electrophoresis of the harvested proteins after human breast cancer tumor cells were treated with Palb-lipo at different concentrations for 24 hours, and the results show that Palb-lipo can effectively degrade target protein. FIG. 57 is Av45-lipo structural schematic diagram and AV45-SH structure. FIG. 58 shows confocal microscopy imaging of human glial cells treated with AV45-lipo at different concentrations for 24 hours, i.e., detection of effect of hijacking β-amyloid (Aβ) into cells. 10 μM oligomers prepared from FITC green dye-labeled β-amyloid 1-42 polypeptide and 10 μM AV45-lipo were co-incubated for 24 hours, then, treated matters not entering the cell were washed off with PBS, followed by confocal microscope imaging, to detect green fluorescence of the β-amyloid 1-42 oligomers. Strong green fluorescence indicates that more β-amyloids are hijacked into the cells, and theoretically, more hijacking and phagocytosis and more lysosome transfers can bring about better degradation, so as to reduce toxicity caused by extracellular β-amyloid oligomer accumulation. The lysosomal dye lysoTracker was used to label the lysosome. The scale bar was 10 μm.

### Example 11

With reference to Table 7, in the present example, the degradation tool in Example 11 was prepared, including coupling the POI recognition groups with the amphiphilic lipid linkers, and subsequently self-assembling the coupled POI recognition groups and the amphiphilic lipid linkers with the lipid hybrid substances. The POI recognition groups therein were monoclonal antibody drug nimotuzumab (NTZ); and the linkers were NHS-PEG-DSPE or NHS-PEG-DMG(1,2-dimyristoyl-rac-glycerol-3-methoxypolyethylene glycol, and N-hydroxysuccinimide is modified at the terminal of polyethylene glycol) with NHS group. The lipid hybrid substances were composed of membrane skeleton HSPC, cholesterol, and the cationic lipid DOTAP (1,2-dioleoyloxy-3-(trimethylammonium)propane). The cationic lipid DOTAP is a representative cationic lipid. The cationic lipid helps the lipid hybrid substances to form lipid nanoparticles (LNP) for entrapping negatively charged nucleic acid drugs. When the composition of the lipid hybrid substance is PEG-DSPE/cholesterol/HSPC, the degradation tool formed is NTZ-LNP1, and when NTZ-LNP1 entraps nonsense sequence non-loaded small interfering RNA (siRNA), the degradation tool formed is NTZ-LNP1s; when the composition of the lipid hybrid substance is PEG-DMG/cholesterol/HSPC, the degradation tool formed is NTZ-LNP2.

Preparation Method:

Pretreatment thereof, linking of the POI recognition groups to the linkers, and purification employed the method used in Example 10.

Preparation of the lipid hybrid substances: the lipid hybrid substances used were lipid nanoparticles, composed of hydrogenated soy phosphatidylcholine (HSPC) or distearoyl phosphatidylcholine (DSPC), cholesterol (CHO), and DSPE-PEG (2 kDa) by the classical membrane hydration method, wherein a mass ratio of [HSPC or DSPC]:CHO:DSPE-PEG (not containing POI recognition group)=56:39:2.5. HSPC and CHO were dissolved in chloroform, and then evaporated, concentrated, and dried to form a membrane.

NTZ-LNP1: After the abovementioned lipid hybrid substances were synthesized, when the chloroform was completely evaporated, 1 mL of PBS solution of DSPE-PEG-antibody was added, and meanwhile DOTAP solution (molar ratio HSPC:DOTAP=1:1) was added, followed by ultrasonic treatment at 100 W for 3 min at room temperature. The resulting solution was passed through 800 nm, 400 nm, and 200 nm filter membranes, respectively.

NTZ-LNP1s: after the abovementioned lipid hybrid substances were synthesized, when the chloroform was completely evaporated, 1 mL of PBS solution of DSPE-PEG-antibody in was added, and the amount of siRNA was calculated according to the ratio of nitrogen to phosphorus (N/P=3:1) of DOTAP. DOTAP solution (molar ratio HSPC:DOTAP=1:1) and siRNA solution were added at the same time. Then the resultant mixture was subjected to ultrasonic treatment at 100 W for 3 min at room temperature. The resulting solution was passed through 800 nm, 400 nm, and 200 nm filter membranes, respectively.

(3) Purification: reaction mixture was then subjected to ultrafiltration concentration by a kDa centrifuging ultrafiltration tube for 3 times, followed by dosing to 100 μL with PBS. Protein concentration was determined in IgG mode by NanoDrop one C instrument according to the absorbance at A280, and the result thereof was verified by a BCA protein quantification kit.

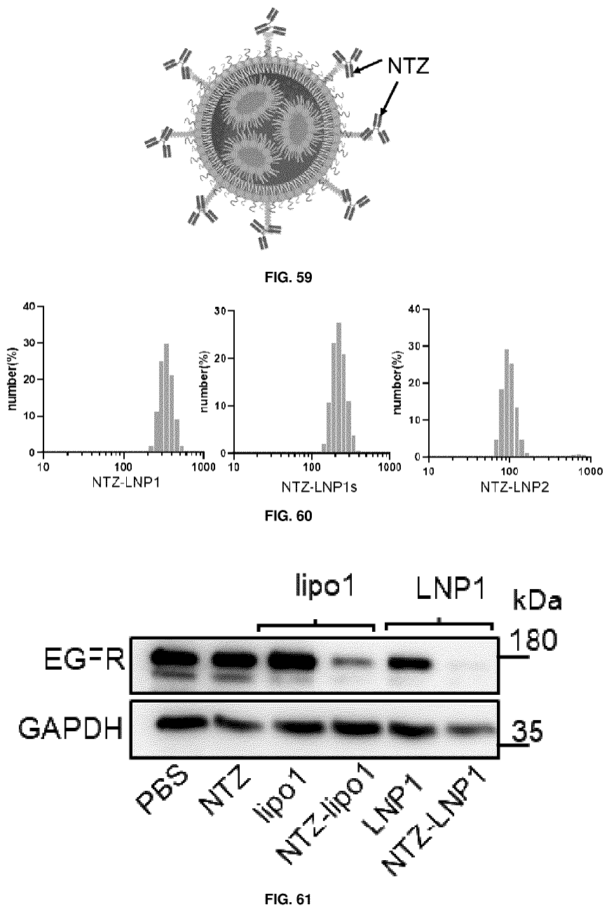

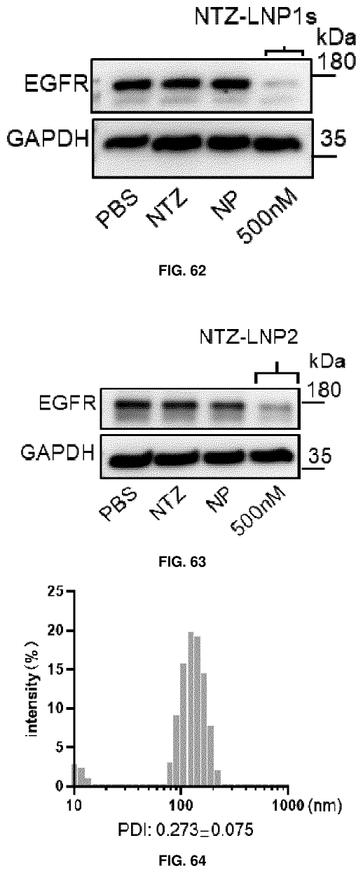

Experimental results: FIG. 59 is a structural schematic diagram of NTZ-LNP1, NTZ-LNP1s, and NTZ-LNP2. FIG. 60 shows particle size distribution of the lipid hybrid substances detected by dynamic light scattering (DLS). FIG. 61 shows results of EGFR protein expression detected by protein electrophoresis of the harvested proteins after M231 human breast cancer tumor cells were treated with 500 nM NTZ-LNP1 for 24 hours, wherein lipo1 in Example 10 also serves as a control. Results show that NTZ-LNP1 can effectively degrade the target protein EGFR. FIG. 62 shows results of EGFR protein expression detected by protein electrophoresis of the harvested proteins after M231 human breast cancer tumor cells were treated with NTZ-LNP1s for 24 hours, and results show that NTZ-LNP1s can effectively degrade the target protein EGFR. FIG. 63 shows results of EGFR protein expression detected by protein electrophoresis of the harvested proteins after M231 human breast cancer tumor cells were treated with NTZ-LNP2 for 24 hours, and results show that NTZ-LNP2 can effectively degrade the target protein EGFR.

### Example 12

With reference to Table 7, in the present example, the degradation tool in Example 12 was prepared, including coupling the POI recognition groups with the amphiphilic lipid linkers, and subsequently self-assembling the coupled POI recognition groups and the amphiphilic lipid linkers with the lipid hybrid substances. The POI recognition groups therein were monoclonal antibody drug nimotuzumab (NTZ); and the linkers were NHS-PEG-DSPE with NHS group. The lipid hybrid substances were composed of exosomes. The exosome is an extracellular vesicle secreted by cell of a eukaryotic organism, such as an animal or a plant, is formed spontaneously, can perform intracellular communication, and generally contains proteins and a small amount of nucleic acids. The exosome has relatively high biocompatibility and drug-carrying capacity, and has a surface of a phospholipid membrane structure.

Preparation Method:

Pretreatment thereof, linking of the POI recognition groups to the linkers, and purification employed the method used in Example 10.

Preparation of the lipid hybrid substances: exosomes from DC2.4 cells were separated, purified, and identified according to standard steps, and the exosomes were coupled with the linkers by two methods, namely, ultrasonic method and membrane extrusion. That is, 1 mL of PBS solution of DSPE-PEG-antibody was added to the exosome PBS solution.

The membrane extrusion or ultrasonic method was adopted for the binding of the linkers and the lipid hybrid substances. The ultrasonic method is to add the PBS solution of DSPE-PEG-antibody to the PBS solution of exosomes (for example, mass ratio of exosomes:DSPE-PEG=1 mg:25 μg in 1 mL system, i.e., 40:1), perform ultrasonic treatment for 30 seconds, and then perform incubation at 37° C. for 1 hour. The membrane extrusion method is to add 1 mL of PBS solution of DSPE-PEG-antibody (mass ratio of exosomes:DSPE-PEG=1 mg:25 μg, i.e., 40:1) to the exosomes and then perform ultrasonic treatment for at 100 W for 3 min at room temperature. The resulting solution was then passed through 800 nm, 400 nm, and 200 nm filter membranes, respectively.

Purification: reaction mixture was then subjected to ultrafiltration concentration by a 50 kDa centrifuging ultrafiltration tube for 3 times, followed by dosing to 100 μL with PBS. Protein concentration was determined in IgG mode by NanoDrop one C instrument according to the absorbance at A280, and the result thereof was verified by a BCA protein quantification kit.

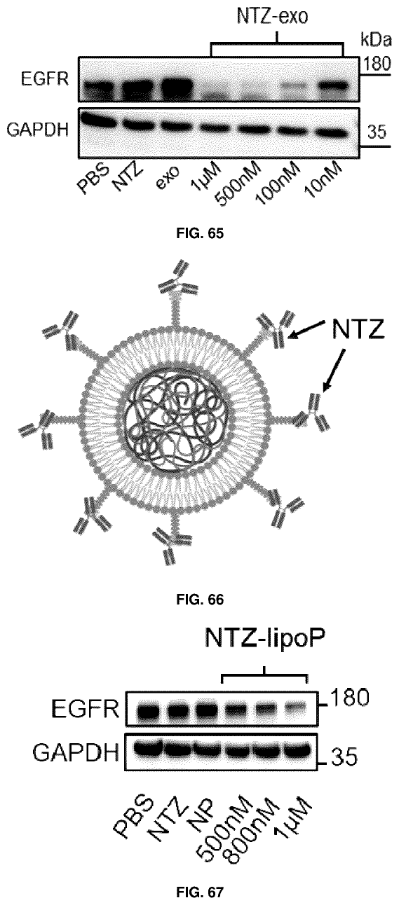

Experimental results: FIG. 64 shows the particle size and average dispersion coefficient PDI (polymer dispersion index) of the lipid hybrid substance detected by DLS, in which the smaller the PDI is, the more uniform the particle size is, and the results show that the particle size is uniform. FIG. 65 shows results of EGFR protein expression detected by protein electrophoresis of the harvested proteins after M231 human breast cancer tumor cells were treated with NTZ-exo at different concentrations for 24 hours, and results show that NTZ-exo can effectively degrade the target protein EGFR.

### Example 13

With reference to Table 7, in the present example, the degradation tool in Example 13 was prepared, i.e., coating the lipid hybrid substance on the surface of the nanoparticles, coupling the POI recognition groups with the amphiphilic lipid linkers, and subsequently self-assembling the coupled POI recognition groups and the amphiphilic lipid linkers with the lipid hybrid substance, and exposing the POI recognition groups to the outside. The POI recognition groups therein were EGFR-targeted monoclonal antibody drug nimotuzumab (NTZ) or Cetuximab (CTX); and the linkers were NHS-PEG-DSPE with NHS group. When the lipid hybrid substances were composed of HSPC/cholesterol and wrapped the PLGA nanoparticles, the degradation tool was NTZ-lipoP. A polylactic acid-glycolic acid (PLGA) copolymer was a representative polymer nanoparticle, which is hydrophobic, has relatively high biocompatibility and stable properties, can be degraded by organisms, and is widely used for medical treatment. Where the lipid hybrid substances were composed of red blood cell membranes (RBCm) and wrapped acetalized dextran (Dextran), and the POI recognition groups were composed of CTX, the degradation tool was CTX-RBCmD. Dextran is a representative hydrophilic biodegradable nanoparticle. The red blood cell membrane is a representative of cell membrane and organelle membrane, and is a lipid hybrid substance, facilitating isolation and extraction.

Preparation Method:

Pretreatment thereof, linking of the POI recognition groups to the linkers, and purification employed the method used in Example 10.

Preparation of the Lipid Hybrid Substance:

Preparation of NTZ-lipoP: composed of HSPC and CHO by the classical membrane rehydration method, in which various compounds had a mass ratio of HSPC:CHO:PEG-DSPE (without POI recognition group)=56:39:2.5. HSPC and CHO were dissolved in chloroform, and then concentrated and evaporated to form the lipid membrane. After the chloroform was completely evaporated, 1 mL of antibody-PEG-DSPE dissolved in PBS was added for resuspension, followed by ultrasonic treatment at 100 W for 3 min at room temperature. The resulting solution was then passed through 800 nm, 400 nm, and 200 nm filter membranes, respectively, to insert DSPE-PEG-antibody into the lipid membrane.

Wrap PLGA: PLGA (15 kDa) was dissolved in DMF (10 μg/μL), 2 μL of the resultant mixture was added to 1 mL of PBS solution every 10 seconds, the resultant mixture was stirred at 700 rpm for 3 h, after DMF was evaporated, each 0.01 μmol of PLGA was wrapped with 1 mg of LIPO on the outside (which means that HSPC+CHO is 1 mg), an appropriate amount of HSPC and CHO were dissolved in chloroform, and after evaporation and concentration, the resultant mixture was resuspended with a PBS solution of DSPE-PEG-NTZ, followed by ultrasonic treatment at 100 W for 3 min. The resultant was subjected to co-incubation on a shaker at 37° C. for 30 minutes in total, ultrasonic treatment at 100 W at room temperature for 5 min, and passed through 800, 400, and 200 nm membranes. The mixture of the two was then mixed with PBS solution of PLGA, followed by 100 W ice bath and ultrasonic treatment for 2 min, passing through 800, 400, and 200 nm membranes, ultrafiltration concentration, and IgG concentration detection.

Preparation of CTX-RBCmD: CTX-PEG-DSPE Synthesis Method is as in Example 10.

1 mg of acetalized dextran was dissolved in 200 μL of tetrahydrofuran, the resultant was dropwise added, 10 μL for each drop, to 1 mL of pH 8 base water under stirring, stirred at 700 rpm for 3 hours. 1 mg of mouse red blood cell membrane was taken and resuspended in 1 mL of PBS. After the PBS solution of the red blood cell membrane was mixed with a PEG solution of CTX-PEG-DSPE (calculated according to the membrane: acetalized dextran=1 mg: 1 mg, the molar ratio of the acetalized dextran core to the DSPE-PEG is 10:1), the mixture was incubated on a shaker at 37° C. at 200 rpm for 30 min, followed by ultrasonic treatment at 100 W at room temperature for 5 min, then, the resulting solution was filtered through 400 nm and 200 nm filter membranes sequentially to obtain a uniform dimension. The resultant was then mixed with an acetalized dextran solution, followed by 100 W ice bath and ultrasonic treatment for 2 min, and the resulting solution was filtered through 400 nm and 200 nm filter membranes, respectively, to obtain a target product. Then ultrafiltration concentration was carried out. Protein concentration was determined in IgG mode by NanoDrop one C (ThermoFisher) according to the absorbance at A280, and the result thereof was verified through by a BCA protein quantification kit.

TABLE 7
DLS Particle Size and PDI Statistical Results of NTZ-lipoP
and Lipo-PLGA without NTZ-PEG-DSPE linkage

| Item | | |
|---|---|---|
| Name | SIZE | PDI |
| lipo-PLGA | 222.1 ± 5.4 | 0.203333 ± 0.032 |
| NTZ-lipoP | 257 ± 7.0 | 0.279667 ± 0.018 |

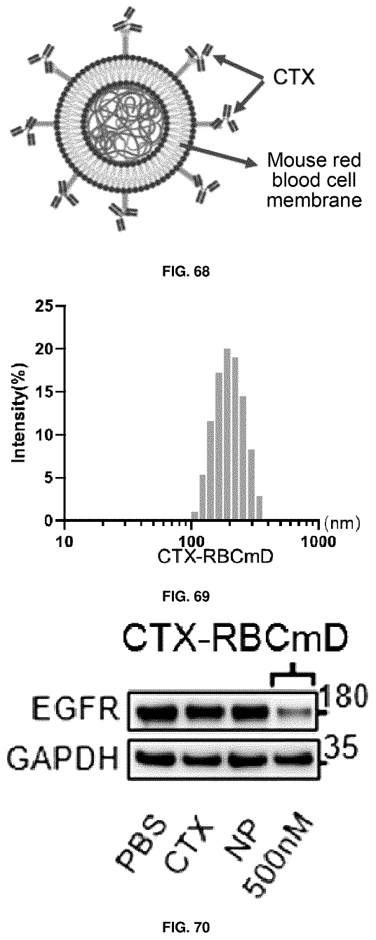

Experimental results: FIG. 66 is a structural schematic diagram of NTZ-lipoP. Table 7 shows DLS particle size and PDI statistical results of NTZ-lipoP and lipo-PLGA without NTZ-PEG-DSPE linkage, and the results show that the particle size is uniform, and the hydrated particle size slightly increases after linking with the NTZ-PEG-DSPE. FIG. 67 shows EGFR protein expression results detected by protein electrophoresis of the harvested proteins after M231 human breast cancer tumor cells were treated with NTZ-lipoP at different concentrations for 24 hours, and the results show that it can effectively degrade the target protein EGFR. FIG. 68 is a structural schematic diagram of CTX-RBCmD. FIG. 69 is a diagram of DLS particle size distribution results. FIG. 70 shows EGFR protein expression results detected by protein electrophoresis of the harvested proteins after M231 human breast cancer tumor cells were treated with 500 nM CTX-RBCmD for 24 hours, and the results show that it can effectively degrade the target protein EGFR.

### INDUSTRIAL APPLICABILITY

In the present disclosure, by modifying the POI recognition groups on surfaces of the nanoparticles, or linking the POI recognition groups to the linkers, or modifying the POI recognition groups on the linkers and linking the POI recognition groups to the surfaces of the nanoparticles through the linkers, after assembly, the POI recognition groups are exposed to the outside of the nanoparticles or the linkers, wherein the nanoparticles (NP) can permeate cells without depending on specially designed receptor-ligand matching pairs, and can be coupled with the POI recognition groups such as small molecules, peptides, nucleic acid aptamers, and antibodies, and specifically perform protein hijacking and targeted degradation, so as to realize the assembly of the TPD tool (TPD-NP) based on the nanoparticles (NP). Such convenient nanoparticle-based TPD tool is extremely easy to obtain drug carrying and tissue-specific targeting capacity, and enables drug and protein degradation combined treatment and transformation/precision medicine to become possible. The invention of TPD-NP and exploration of mechanism thereof greatly expand the range of the TPD tool, provide basic knowledge for the TPD and the field of nano delivery, and in principle, can degrade in vivo extracellular/membrane-related/intracellular proteins related to a variety of human diseases.

The present disclosure systematically proposes, for the first time, nanoparticle-mediated protein degradation, providing a new path for TPD and nano delivery. The nano-structural protein degradation tool of the present disclosure has a flexible structure, is convenient to modify, and can obtain the capabilities of drug carrying, targeting, and crossing biological barriers. The nano-structural protein degradation tool of the present disclosure has universality, a target can be randomly changed, all of the three components can be replaced, and the use scenarios are wide. All of the components of the nano-structural protein degradation tool of the present disclosure can be clinically approved materials, have a high potential for in vivo use, and have a translational medicine value. As a ready-to-use platform, the nano-structural protein degradation tool of the present disclosure does not require de novo synthesis from the raw chemical materials, thus greatly reducing the complexity and difficulty of development and production. Compared with that the PROTAC degrades intracellular proteins, and LYTAC degrades extracellular/membrane proteins, the TPD-NP can degrade extracellular/intracellular/membrane proteins. In addition, with respect to extracellular/membrane protein degradation tools such as LYTAC, the TPD-NP does not need to be additionally designed with a structure for assisting the hijacked proteins to be endocytosed. Compared with the existing TPD tools, the nano-structural protein degradation tool of the present disclosure does not need a special structure to guide protein degradation. Nanoparticles can carry drugs, can be designed to be controllably released, can be designed to be photothermomagnetic and other synergistic treatment materials, and can be imaged and contrasted to further carry out synergistic treatment and integrated diagnosis and treatment of protein degradation. The humoral stability of NP can reduce drug loss and improve the pharmaceutical potential.

In addition, in the present disclosure, by modifying the POI recognition groups on the surfaces of the lipid hybrid substances, and after assembly, exposing the POI recognition groups to the outside of the lipid hybrid substances, the targeted protein degradation based on the lipid hybrid substances is realized, so that the synthesis difficulty of constructing the protein degradation tool is greatly reduced. By means of the “plug and play” mode, massive compounds, peptides, antibodies, nucleic acid aptamers, etc. having a binding capacity to the target protein can be upgraded to be protein degradation drugs, to further play a new role in the fields related to conventional liposome and lipid nanoparticle (LNP), such as mRNA vaccines, nucleic acid delivery carriers, and drug delivery, thus realizing combined therapy development in scientific research and industrial applications.

The combination manner of “plug and play” adopted in the present disclosure is extremely convenient, the structure can be flexibly assembled as required, the assembling materials can be completely from clinically acceptable materials, the nanocarrier is creatively endowed with the protein degradation function other than the delivery property, and the scope of biotechnology drugs is broadened.

Moreover, the search for the use of ligand-targeted lipid nanoparticles (lipid hybrid substances) as protein degradation tool and the mechanism thereof is still blank. The present disclosure greatly extends the current use range of lipid nanoparticles, provides basic knowledge for the fields of TPD and nano delivery, and can, in principle, degrade extracellular/membrane-related/intracellular proteins associated with various human diseases in vivo.

The abovementioned are preferred embodiments and corresponding examples of the present disclosure, and it should be indicated that those ordinarily skilled in the art further could make several variations and improvements, without departing from the inventive concept of the present disclosure, including but not limited to adjustment to ratios, procedures, and dosages, and all of these should be within the scope of protection of the present disclosure.

What is claimed is:

1. A nano-structural protein degradation tool, comprising: one of or a combination of several of a first degradation tool, a second degradation tool, and a third degradation tool, wherein
the first degradation tool is formed by linking protein of interest (POI) recognition groups to linkers; the second degradation tool is formed by linking the POI recognition groups to a nanoparticle; and the third degradation tool is formed by linking the POI recognition groups to the nanoparticle through the linkers; and
the POI recognition groups comprise antibodies, proteins, peptides, nucleic acid aptamers or small molecules capable of specifically binding to the POI.

2. The nano-structural protein degradation tool according to claim 1, wherein
in the first degradation tool, the POI recognition group and the linker constitute a set of linking unit; and the first degradation tool is the nano-structural protein degradation tool having a multi-layer structure and composed of a plurality of sets of linking units, wherein the linking unit comprises the linker located at a core and the POI recognition group linked to the linker and located at periphery;
the second degradation tool is the nano-structural protein degradation tool having a multi-layer structure and composed of the nanoparticle located at the core and a plurality of the POI recognition groups linked to the nanoparticle and located at periphery; and
in the third degradation tool, the POI recognition groups are linked to the nanoparticle through the linkers; and the third degradation tool is the nano-structural protein degradation tool having a multi-layer structure and composed of the nanoparticle located at the core and a plurality of sets of linking units, wherein the linking unit comprises the linker linked to the nanoparticle and located at an intermediate layer, and the POI recognition group located at periphery and linked to the linker.

3. The nano-structural protein degradation tool according to claim 1, wherein
the linkers comprise hydrophilic polymers, hydrophobic polymers, and amphiphilic polymers;
the linkers have molecular weights of 0-1000 kDa, not including 0 kDa; and
the amphipathic polymers comprise: amphipathic block co-polymers, amphipathic polymers formed by the hydrophilic polymers and hydrophobic small molecules, and amphipathic polymers formed by the hydrophobic polymers and hydrophilic small molecules.

4. The nano-structural protein degradation tool according to claim 3, wherein the amphiphilic polymers are polymers of chain-like or branched molecular structures, which have at least one hydrophilic molecular terminal and one hydrophobic molecular terminal.

5. The nano-structural protein degradation tool according to claim 4, wherein the amphiphilic polymers are of straight-chain molecular structures, with one terminal being a hydrophilic molecular terminal and the other terminal being a hydrophobic molecular terminal.

6. The nano-structural protein degradation tool according to claim 3, wherein the nanoparticle comprises surface single nanoparticle and hybrid nanoparticle;
the surface single nanoparticle comprises hydrophilic particle, hydrophobic particle, and inorganic nanoparticle;
the hybrid nanoparticle is a hybrid nanoparticle obtained by modifying the surface single nanoparticle with a hybrid substance;
wherein the hybrid substance is a modified membrane; and the modified membrane comprises cell membrane, exosome, oil membrane, hydrogel, and liposome;
the hybrid nanoparticle is a particle formed by coating an outer surface of the surface single nanoparticle with the modified membrane, so that the hybrid nanoparticle modified from the surface single nanoparticle can be linked to arms of the hydrophilic polymers, arms of the hydrophobic polymers, or the POI recognition groups; and
the nanoparticle has a particle size of 5-1000 nm.

7. The nano-structural protein degradation tool according to claim 6, wherein
in the third degradation tool, the nanoparticle is hydrophobic particle, hydrophilic particle, or the surface single nanoparticle is coated with the modified membrane, and when the linkers are the amphiphilic polymers, the hydrophilic polymers or the hydrophobic polymers, methods of linking the linkers to the nanoparticle comprise: non-covalently bonding the linkers to the nanoparticle, or covalently bonding the linkers to the nanoparticle through reactive groups modified on the linkers.

8. The nano-structural protein degradation tool according to claim 1, wherein
the antibodies in the POI recognition groups are therapeutic monoclonal antibodies, multispecific antibodies, nanobodies or derivatives or antibody-drug conjugates of the preceding antibodies;
the peptides are peptides having specific POI binding capacity; and
the small molecules are small molecule compounds having specific POI binding capacity.

9. A method for treatment and prevention of diseases of abnormal protein accumulation, comprising administering to a subject a therapeutically effective amount of the nano-structural protein degradation tool according to claim 1, wherein the diseases of abnormal protein accumulation comprise tumors, immune system diseases, inflammations, pathogen infections, neurodegenerative diseases, blood system diseases, and metabolic diseases.

10. A lipid-based protein degradation tool, comprising:
POI recognition groups, and lipid hybrid substance linked to the POI recognition groups, wherein the POI recognition groups comprise antibodies, proteins, peptides, nucleic acid aptamers or small molecules capable of specifically binding to the POI; and
the lipid hybrid substance comprises liposome, exosome, cell membrane and LNP.

11. The lipid-based protein degradation tool according to claim 10, wherein
when the POI recognition groups are coupled with the lipid hybrid substance, the lipid-based protein degradation tool is nanoparticle composed of the lipid hybrid substance at a core and the POI recognition groups located at periphery for protein degradation.

12. The lipid-based protein degradation tool according to claim 10, wherein
the lipid-based protein degradation tool further comprises the lipid-based protein degradation tool provided with linking members between the POI recognition groups and the lipid hybrid substance;
the linking members have a molecular weight of 0-1000 kDa, not including 0 kDa;
the linking members are one of polymer linkers and lipid linkers;
the polymer linkers comprise hydrophilic polymers, hydrophobic polymers, and amphiphilic polymers; and
the lipid linkers are amphiphilic lipid linkers.

13. The lipid-based protein degradation tool according to claim 12, wherein when the POI recognition groups are coupled with the lipid hybrid substance through the linking members, the POI recognition group and the linking member constitute a set of linking unit; the lipid-based protein degradation tool is the lipid-based protein degradation tool having a multi-layer structure and composed of the lipid hybrid substance located at the core and a plurality of sets of linking units, wherein the linking unit comprises the linker located at an intermediate layer and linked to the lipid hybrid substance, and the POI recognition group located at periphery and linked to the linking member.

14. The lipid-based protein degradation tool according to claim 12, wherein the lipid linker comprises at least two terminals, one terminal being a lipophilic terminal capable of being linked to the lipid hybrid substance, and the other terminal being a hydrophilic terminal; and
the lipophilic terminal is a lipid molecule.

15. The lipid-based protein degradation tool according to claim 12, wherein
the amphiphilic polymers are polymers of chain-like or branched molecular structures, which have at least one hydrophilic molecular terminal and one hydrophobic molecular terminal.

16. The lipid-based protein degradation tool according to claim 15, wherein the amphiphilic polymers are of straight-chain molecular structures, with one terminal being a hydrophilic molecular terminal and the other terminal being a hydrophobic molecular terminal.

17. The lipid-based protein degradation tool according to claim 10, further comprising nanoparticle, wherein the nanoparticle is coated by the lipid hybrid substance at the core of the lipid-based protein degradation tool;
wherein the nanoparticle comprises hydrophilic particle, hydrophobic particle, and inorganic nanoparticle; and
the nanoparticle has a particle size of 5-1000 nm.
